(19) United States
(12) Patent Application Publication
(10) Pub. No.: US 2026/0317414 A1
(43) Pub. Date: Oct. 15, 2026

(54) IL17A ANTIBODIES AND ANTAGONISTS FOR VETERINARY USE

(71) Applicant: Elanco US Inc., Indianapolis, IN (US)

(72) Inventors: Shyr Jiann LI, Indianapolis, IN (US); Lam NGUYEN, Indianapolis, IN (US); Lan YANG, Indianapolis, IN (US); Hangjun ZHAN, Indianapolis, IN (US)

(21) Appl. No.: 19/554,121

(22) Filed: Mar. 2, 2026

Related U.S. Application Data
(63) Continuation of application No. 17/687,083, filed on Mar. 4, 2022, now Pat. No. 12,583,917.
Application No. 17/687,083 is a continuation of application No. 16/621,012, filed on Jun. 18, 2018, now Pat. No. 11,299,540.
(60) Provisional application No. 62/521,514, filed on Jun. 18, 2017.

(51) Int. Cl. C07K 16/24; A61K 39/00; A61K 39/395; C12N 15/63

(52) U.S. Cl. CPC C07K 16/244; A61K 39/3955; C12N 15/63; A61K 2039/505; C07K 2317/24; C07K 2317/31; C07K 2317/33; C07K 2317/56; C07K 2317/565; C07K 2317/76

(57) ABSTRACT

Provided are various embodiments relating to anti-IL17A antibodies and IL17Ra ECD polypeptides that bind to IL17A. In various embodiments, such anti-IL17A antibodies or IL17Ra ECD polypeptides can be used in methods to treat IL17-induced conditions in subjects, such as humans or companion animals, such as canines, felines, and equines. Also provided are various embodiments relating to IgG Fc variant polypeptides having one or more amino acid substitutions for reducing binding to C1q and/or CD16. In some embodiments, the IgG Fc variants and/or polypeptides comprising the IgG Fc variants (e.g., fusion polypeptides comprising the IgG Fc variants and the anti-IL17A antibodies and/or IL17Ra ECD polypeptides described herein) may have reduced complement-mediated immune responses and/or antibody-dependent cell-mediated cytotoxicity.

### CROSS-REFERENCE TO RELATED APPLICATIONS

This application is a continuation of U.S. application Ser. No. 17/687,083, filed on 4 Mar. 2022, which is a continuation of U.S. application Ser. No. 16/621,012, filed on 10 Dec. 2019, now U.S. patent Ser. No. 11/299,540, issued on 12 Apr. 2022, which is a national stage application of PCT/US2018/038033, filed on 18 Jun. 2018, which claims the benefit of priority to U.S. Provisional Application No. 62/521,514, filed on 18 Jun. 2017, the entire contents of each of which are incorporated herein by reference.

### REFERENCE TO SEQUENCE LISTING XML

Pursuant to the EFS-Web legal framework and 37 C.F.R. § 1.831-835 (see M.P.E.P. § 2413.03), a Sequence Listing in eXtensible Markup Language (XML) format (entitled “2920951-453003_Sequence_Listing.xml” created on 23 Jun. 2026 and 172,540 bytes in size) is submitted concurrently with the instant application, and the entire contents of the Sequence Listing are incorporated herein by reference.

### FIELD

This present disclosure relates to anti-IL17A antibodies and IL17Ra ECD polypeptides for binding to IL17A, for example canine, feline, and/or equine IL17A, and methods of using the same, for example, treating IL17-induced conditions or reducing IL17 signaling function in cells, for instance in companion animals, such as canines, felines, and equines. The present disclosure also relates to IgG Fc variant polypeptides having one or more amino acid substitutions for reducing binding to C1q and/or CD16 and methods of using the same. For example, IgG Fc variants and/or polypeptides comprising the IgG Fc variants (e.g., fusion polypeptides comprising the IgG Fc variants and the anti-IL17A antibodies and/or IL17Ra ECD polypeptides described herein) may have reduced complement-mediated immune responses and/or antibody-dependent cell-mediated cytotoxicity.

### BACKGROUND

Interleukin 17A (IL17A) is a homodimeric cytokine produced by T helper 17 (Th17) cells and understood to be involved in immune disorders such as plaque psoriasis, psoriatic arthritis, rheumatoid arthritis, airway inflammation, asthma, osteoarthritis, inflammatory bowel disorder, Crohn's disease, ankylosing spondylitis, atopic dermatitis, degenerative myelopathy, multiple sclerosis, and uveitis.

IL17A is understood to function by binding its receptor IL17Ra and activating downstream pathways, such as activation of NFκB, MAPKs, and C/EBPs to induce production of cytokines and chemokines, and induce host defense to microbial infection.

Companion animals such as cats, dogs, and horses suffer from many diseases similar to human diseases. There remains a need for methods and compounds that can be used specifically to bind companion animal IL17A for treating IL17A-induced conditions and for reducing IL17A signaling.

### SUMMARY

Embodiment 1. An isolated antibody that binds to canine IL17A, wherein the antibody binds to an epitope within amino acids 65 to 88 of SEQ ID NO: 22.

Embodiment 2. The antibody of embodiment 1, wherein the antibody binds to an epitope comprising or within the amino acid sequence of SEQ ID NO: 23 or SEQ ID NO: 51.

Embodiment 3. The antibody of embodiment 1 or embodiment 2, wherein the antibody binds to canine IL17A with a dissociation constant (Kd) of less than 5×10-6 M, less than 1×10-6 M, less than 5×10-7 M, less than 1×10-7 M, less than 5×10-8 M, less than 1×10-8 M, less than 5×10-9 M, less than 1×10-9 M, less than 5×10-10 M, less than 1×10-10 M, less than 5×10 11 M, less than 1×10 11 M, less than 5×10-12 M, or less than 1×10-12 M, as measured by biolayer interferometry.

Embodiment 4. The antibody of any one of embodiments 1 to 3, wherein the antibody reduces IL17A signaling function in a companion animal species, as measured bya reduction in IL6 secretion in a cell-based assay.

Embodiment 5. The antibody of embodiment 4, wherein the companion animal species is canine, feline, or equine.

Embodiment 6. The antibody of any one of embodiments 1 to 5, wherein the antibody binds to feline IL17A or equine IL17A as determined by immunoblot analysis or biolayer interferometry.

Embodiment 7. The antibody of any one of embodiments 1 to 6, wherein the antibody competes with monoclonal Clone A, monoclonal Clone C, or monoclonal Clone E antibody in binding to canine IL17A.

Embodiment 8. The antibody of any one of embodiments 1 to 6, wherein the antibody competes with monoclonal Clone A, monoclonal Clone C, or monoclonal Clone E antibody in binding to feline IL17A or in binding to equine IL17A.

Embodiment 9. The antibody of any one of embodiments 1 to 8, wherein the antibody is a monoclonal antibody.

Embodiment 10. The antibody of any one of embodiments 1 to 9, wherein the antibody is a canine, a caninized, a feline, a felinized, an equine, an equinized, or a chimeric antibody.

Embodiment 11. The antibody of any one of embodiments 1 to 10, wherein the antibody is a chimeric antibody comprising murine variable heavy chain framework regions or murine variable light chain framework regions.

Embodiment 12. The antibody of any one of embodiments 1 to 11, comprising a heavy chain and a light chain, wherein:
a. (i) the heavy chain comprises a CDR-H1 sequence having at least 85%, at least 90%, at least 95, or at least 98% sequence identity to the amino acid sequence of the amino acid sequence of SEQ ID NO: 1, a CDR H2 sequence having at least 85%, at least 90%, at least 95, or at least 98% sequence identity to the amino acid sequence of the amino acid sequence of SEQ ID NO: 2, and a CDR-H3 sequence having at least 85%, at least 90%, at least 95, or at least 98% sequence identity to the amino acid sequence of the amino acid sequence of SEQ ID NO: 3, and
b. (ii) the light chain comprises a CDR-L1 sequence having at least 85%, at least 90%, at least 95, or at least 98% sequence identity to the amino acid sequence of the amino acid sequence of SEQ ID NO: 8, a CDR-L2 sequence having at least 85%, at least 90%, at least 95, or at least 98% sequence identity to the amino acid sequence of the amino acid sequence of SEQ ID NO: 9 or SEQ ID NO: 108, and a CDR-L3 sequence having at least 85%, at least 90%, at least 95, or at least 98% sequence identity to the amino acid sequence of the amino acid sequence of SEQ ID NO: 10; or
c. (i) the heavy chain comprises a CDR-H1 sequence having at least 85%, at least 90%, at least 95, or at least 98% sequence identity to the amino acid sequence of the amino acid sequence of SEQ ID NO: 52, a CDR H2 sequence having at least 85%, at least 90%, at least 95, or at least 98% sequence identity to the amino acid sequence of the amino acid sequence of SEQ ID NO: 53 or SEQ ID NO: 109, and a CDR-H3 sequence having at least 85%, at least 90%, at least 95, or at least 98% sequence identity to the amino acid sequence of the amino acid sequence of SEQ ID SEQ ID NO: 54, and
d. (ii) the light chain comprises a CDR-L1 sequence having at least 85%, at least 90%, at least 95, or at least 98% sequence identity to the amino acid sequence of the amino acid sequence of SEQ ID NO: 59 or SEQ ID NO: 111, a CDR-L2 sequence having at least 85%, at least 90%, at least 95, or at least 98% sequence identity to the amino acid sequence of the amino acid sequence of SEQ ID NO: 60 or SEQ ID NO: 112, and a CDR-L3 sequence having at least 85%, at least 90%, at least 95, or at least 98% sequence identity to the amino acid sequence of the amino acid sequence of SEQ ID NO: 61; or
e. (i) the heavy chain comprises a CDR-H1 sequence having at least 85%, at least 90%, at least 95, or at least 98% sequence identity to the amino acid sequence of the amino acid sequence of SEQ ID NO: 66, a CDR H2 sequence having at least 85%, at least 90%, at least 95, or at least 98% sequence identity to the amino acid sequence of the amino acid sequence of SEQ ID NO: 67 or SEQ ID NO: 114, and a CDR-H3 sequence having at least 85%, at least 90%, at least 95, or at least 98% sequence identity to the amino acid sequence of the amino acid sequence of SEQ ID NO: 68, and
f. (ii) the light chain comprises a CDR-L1 sequence having at least 85%, at least 90%, at least 95, or at least 98% sequence identity to the amino acid sequence of the amino acid sequence of SEQ ID NO: 73 or SEQ ID NO: 116, a CDR-L2 sequence having at least 85%, at least 90%, at least 95, or at least 98% sequence identity to the amino acid sequence of the amino acid sequence of SEQ ID NO: 74 or SEQ ID NO: 117; and a CDR-L3 sequence having at least 85%, at least 90%, at least 95, or at least 98% sequence identity to the amino acid sequence of the amino acid sequence of SEQ ID NO: 75.

Embodiment 13. The antibody of any one of embodiments 1 to 12, comprising a heavy chain and a light chain, wherein:
a. (i) the heavy chain comprises a CDR-H1 sequence having the amino acid sequence of SEQ ID NO: 1, a CDR H2 sequence having the amino acid sequence of SEQ ID NO: 2, and a CDR-H3 sequence having the amino acid sequence of SEQ ID NO: 3, and
b. (ii) the light chain comprises a CDR-L1 sequence having the amino acid sequence of SEQ ID NO: 8, a CDR-L2 sequence having the amino acid sequence of SEQ ID NO: 9 or SEQ ID NO: 108, and a CDR-L3 sequence having the amino acid sequence of SEQ ID NO: 10; or
c. (i) the heavy chain comprises a CDR-H1 sequence having the amino acid sequence of SEQ ID NO: 52, a CDR H2 sequence having the amino acid sequence of SEQ ID NO: 53 or SEQ ID NO: 109, and a CDR-H3 sequence having the amino acid sequence of SEQ ID SEQ ID NO: 54, and
d. (ii) the light chain comprises a CDR-L1 sequence having the amino acid sequence of SEQ ID NO: 59 or SEQ ID NO: 111, a CDR-L2 sequence having the amino acid sequence of SEQ ID NO: 60 or SEQ ID NO: 112, and a CDR-L3 sequence having the amino acid sequence of SEQ ID NO: 61; or
e. (i) the heavy chain comprises a CDR-H1 sequence having the amino acid sequence of SEQ ID NO: 66, a CDR H2 sequence having the amino acid sequence of SEQ ID NO: 67 or SEQ ID NO: 114, and a CDR-H3 sequence having the amino acid sequence of SEQ ID NO: 68, and
f. (ii) the light chain comprises a CDR-L1 sequence having the amino acid sequence of SEQ ID NO: 73 or SEQ ID NO: 116, a CDR-L2 sequence having the amino acid sequence of SEQ ID NO: 74 or SEQ ID NO: 117; and a CDR-L3 sequence having the amino acid sequence of SEQ ID NO: 75.

Embodiment 14. The antibody of embodiment 12 or embodiment 13, further comprising:
a. one or more of (i) a variable region heavy chain framework 1 (HC FR1) sequence of SEQ ID NO: 4; (ii) a HC FR2 sequence of SEQ ID NO: 5; (iii) a HC-FR3 sequence of SEQ ID NO: 6; (iv) a HC-FR4 sequence of SEQ ID NO: 7; (v) a variable region light chain framework 1 (LC-FR1) sequence of SEQ ID NO: 11; (vi) an LC FR2 sequence of SEQ ID NO: 12; (vii) an LC FR3 sequence of SEQ ID NO: 13; and/or (vii) an LC-FR4 sequence of SEQ ID NO: 14; or
b. one or more of (i) a variable region heavy chain framework 1 (HC FR1) sequence of SEQ ID NO: 55; (ii) a HC FR2 sequence of SEQ ID NO: 56 or SEQ ID NO: 110; (iii) a HC-FR3 sequence of SEQ ID NO: 57; (iv) a HC-FR4 sequence of SEQ ID NO: 58; (v) a variable region light chain framework 1 (LC-FR1) sequence of SEQ ID NO: 62; (vi) an LC FR2 sequence of SEQ ID NO: 63 or SEQ ID NO: 113; (vii) an LC FR3 sequence of SEQ ID NO: 64; and/or (vii) an LC-FR4 sequence of SEQ ID NO: 65; or
c. one or more of (i) a variable region heavy chain framework 1 (HC FR1) sequence of SEQ ID NO: 69; (ii) a HC FR2 sequence of SEQ ID NO: 70 or SEQ ID NO: 115; (iii) a HC-FR3 sequence of SEQ ID NO: 71; (iv) a HC-FR4 sequence of SEQ ID NO: 72; (v) a variable region light chain framework 1 (LC-FR1) sequence of SEQ ID NO: 76; (vi) an LC FR2 sequence of SEQ ID NO: 77 or SEQ ID NO: 118; (vii) an LC FR3 sequence of SEQ ID NO: 78; and/or (vii) an LC-FR4 sequence of SEQ ID NO: 79.

Embodiment 15. The antibody of any one of embodiments 1 to 14, wherein the antibody comprises:
a. (i) a variable light chain sequence having at least 85%, at least 90%, at least 95%, or at least 98% sequence identity to the amino acid sequence of SEQ ID NO: 24; (ii) a variable heavy chain sequence having at least 85%, at least 90%, at least 95%, or at least 98% sequence identity to the amino acid sequence of SEQ ID NO: 25; or (iii) a variable light chain sequence as in (i) and a variable heavy chain sequence as in (ii); or
b. (i) a variable light chain sequence having at least 85%, at least 90%, at least 95%, or at least 98% sequence identity to the amino acid sequence of SEQ ID NO: 16; (ii) a variable heavy chain sequence having at least 85%, at least 90%, at least 95%, or at least 98% sequence identity to the amino acid sequence of SEQ ID NO: 15; or (iii) a variable light chain sequence as in (i) and a variable heavy chain sequence as in (ii); or
c. (i) a variable light chain sequence having at least 85%, at least 90%, at least 95%, or at least 98% sequence identity to the amino acid sequence of SEQ ID NO: 34; (ii) a variable heavy chain sequence having at least 85%, at least 90%, at least 95%, or at least 98% sequence identity to the amino acid sequence of SEQ ID NO: 35; or (iii) a variable light chain sequence as in (i) and a variable heavy chain sequence as in (ii); or
d. (i) a variable light chain sequence having at least 85%, at least 90%, at least 95%, or at least 98% sequence identity to the amino acid sequence of SEQ ID NO: 38; (ii) a variable heavy chain sequence having at least 85%, at least 90%, at least 95%, or at least 98% sequence identity to the amino acid sequence of SEQ ID NO: 39; or (iii) a variable light chain sequence as in (i) and a variable heavy chain sequence as in (ii).

Embodiment 16. The antibody of any one of embodiments 1 to 15, wherein the antibody comprises a variable light chain sequence of SEQ ID NO: 24; SEQ ID NO: 16; SEQ ID NO: 34; or SEQ ID NO: 38.

Embodiment 17. The antibody of any one of embodiments 1 to 16, wherein the antibody comprises a variable heavy chain sequence of SEQ ID NO: 25, SEQ ID NO: 15; SEQ ID NO: 35; or SEQ ID NO: 39.

Embodiment 18. The antibody of any one of embodiments 1 to 17, wherein the antibody comprises:
a. a variable light chain sequence of SEQ ID NO: 24 and a variable heavy chain sequence of SEQ ID NO: 25; or
b. a variable light chain sequence of SEQ ID NO: 16 and a variable heavy chain sequence of SEQ ID NO: 15; or
c. a variable light chain sequence of SEQ ID NO: 34 and a variable heavy chain sequence of SEQ ID NO: 35; or
d. a variable light chain sequence of SEQ ID NO: 38 and a variable heavy chain sequence of SEQ ID NO: 39.

Embodiment 19. The antibody of any one of embodiments 1 to 18, wherein the antibody comprises a constant heavy chain region or constant light chain region derived from a companion animal.

Embodiment 20. The antibody of any one of embodiments 1 to 19, wherein the antibody comprises (a) a canine heavy chain constant region selected from an IgG-A, IgG-B, IgG-C, and IgG-D constant region; (b) a feline heavy chain constant region selected from an IgG-1a, IgG-1 b, and IgG-2 constant region; or (c) an equine heavy chain constant region selected from an IgG-1, IgG-2, IgG-3, IgG-4, IgG-5, IgG-6, and IgG-7 constant region.

Embodiment 21. The antibody of any one of embodiments 1 to 20, wherein the antibody comprises a heavy chain amino acid sequence of SEQ ID NO: 17, SEQ ID NO: 18, SEQ ID NO: 19, SEQ ID NO: 20, or SEQ ID NO: 27.

Embodiment 22. The antibody of any one of embodiments 1 to 21, wherein the antibody comprises a light chain amino acid sequence of SEQ ID NO: 21 or SEQ ID NO: 26.

Embodiment 23. An isolated antibody that binds to feline IL17A, wherein the antibody binds to feline IL17A with a dissociation constant (Kd) of less than 5×10-6 M, less than 1×10-6 M, less than 5×10-7 M, less than 1×10-7 M, less than 5×10-8 M, less than 1×10-8 M, less than 5×10-9 M, less than 1×10-9 M, less than 5×10-10 M, less than 1×10-10 M, less than 5×10 11 M, less than 1×10 11 M, less than 5×10-12 M, or less than 1×10-12 M, as measured by biolayer interferometry.

Embodiment 24. The antibody of embodiment 23, wherein the antibody reduces IL17A signaling function in a companion animal species, as measured by a reduction in IL6 secretion in a cell-based assay.

Embodiment 25. The antibody of embodiment 24, wherein the companion animal species is canine, feline, or equine.

Embodiment 26. The antibody of any one of embodiments 23 to 25, wherein the antibody binds to canine IL17A or equine IL17A as determined by immunoblot analysis or biolayer interferometry.

Embodiment 27. The antibody of any one of embodiments 23 to 26, wherein the antibody competes with monoclonal Clone D in binding to feline IL17A.

Embodiment 28. The antibody of any one of embodiments 23 to 27, wherein the antibody competes with monoclonal Clone D in binding to canine IL17A or in binding to equine IL17A.

Embodiment 29. The antibody of any one of embodiments 23 to 28, wherein the antibody is a monoclonal antibody.

Embodiment 30. The antibody of any one of embodiments 23 to 29, wherein the antibody is a canine, a caninized, a feline, a felinized, an equine, an equinized, or a chimeric antibody.

Embodiment 31. The antibody of any one of embodiments 23 to 30, wherein the antibody is a chimeric antibody comprising murine variable heavy chain framework regions or murine variable light chain framework regions.

Embodiment 32. The antibody of any one of embodiments 23 to 31, comprising a heavy chain and a light chain, wherein:
a. the heavy chain comprises a CDR-H1 sequence having at least 85%, at least 90%, at least 95, or at least 98% sequence identity to the amino acid sequence of the amino acid sequence of SEQ ID NO: 80, a CDR H2 sequence having at least 85%, at least 90%, at least 95, or at least 98% sequence identity to the amino acid sequence of the amino acid sequence of SEQ ID NO: 81 or SEQ ID NO: 119, and a CDR-H3 sequence having at least 85%, at least 90%, at least 95, or at least 98% sequence identity to the amino acid sequence of the amino acid sequence of SEQ ID NO: 82, and
b. the light chain comprises a CDR-L1 sequence having at least 85%, at least 90%, at least 95, or at least 98% sequence identity to the amino acid sequence of the amino acid sequence of SEQ ID NO: 87 or SEQ ID NO: 121, a CDR-L2 sequence having at least 85%, at least 90%, at least 95, or at least 98% sequence identity to the amino acid sequence of the amino acid sequence of SEQ ID NO: 88 or SEQ ID NO: 122, and a CDR-L3 sequence having at least 85%, at least 90%, at least 95, or at least 98% sequence identity to the amino acid sequence of the amino acid sequence of SEQ ID NO: 89.

Embodiment 33. The antibody of any one of embodiments 23 to 32, comprising a heavy chain and a light chain, wherein:
a. the heavy chain comprises a CDR-H1 sequence having the amino acid sequence of SEQ ID NO: 80, a CDR H2 sequence having the amino acid sequence of SEQ ID NO: 81 or SEQ ID NO: 119, and a CDR-H3 sequence having the amino acid sequence of SEQ ID NO: 82, and
b. the light chain comprises a CDR-L1 sequence having the amino acid sequence of SEQ ID NO: 87 or SEQ ID NO: 121, a CDR-L2 sequence having the amino acid sequence of SEQ ID NO: 88 or SEQ ID NO: 122, and a CDR-L3 sequence having the amino acid sequence of SEQ ID NO: 89.

Embodiment 34. The antibody of embodiment 32 or embodiment 33, further comprising one or more of (a) a variable region heavy chain framework 1 (HC FR1) sequence of SEQ ID NO: 83; (b) a HC FR2 sequence of SEQ ID NO: 84 or SEQ ID NO: 120; (c) a HC-FR3 sequence of SEQ ID NO: 85; (d) a HC-FR4 sequence of SEQ ID NO: 86; (e) a variable region light chain framework 1 (LC-FR1) sequence of SEQ ID NO: 90; (f) an LC FR2 sequence of SEQ ID NO: 91 or SEQ ID NO: 123; (g) an LC FR3 sequence of SEQ ID NO: 92; and/or (h) an LC-FR4 sequence of SEQ ID NO: 93.

Embodiment 35. The antibody of any one of embodiments 23 to 34, wherein the antibody comprises (a) a variable light chain sequence having at least 85%, at least 90%, at least 95%, or at least 98% sequence identity to the amino acid sequence of SEQ ID NO: 36; (b) a variable heavy chain sequence having at least 85%, at least 90%, at least 95%, or at least 98% sequence identity to the amino acid sequence of SEQ ID NO: 37; or (c) a variable light chain sequence as in (a) and a variable heavy chain sequence as in (b).

Embodiment 36. The antibody of any one of embodiments 23 to 35, wherein the antibody comprises (i) a variable light chain sequence of SEQ ID NO: 36, (ii) a variable heavy chain sequence of SEQ ID NO: 37; or (iii) a variable light chain sequence of SEQ ID NO: 36 and a variable heavy chain sequence of SEQ ID NO: 37.

Embodiment 37. The antibody of any one of embodiments 23 to 36, wherein the antibody comprises a constant heavy chain region or constant light chain region derived from a companion animal.

Embodiment 38. The antibody of any one of embodiments 23 to 37, wherein the antibody comprises (a) a canine heavy chain constant region selected from an IgG-A, IgG-B, IgG-C, and IgG-D constant region; (b) a feline heavy chain constant region selected from an IgG-1a, IgG-1 b, and IgG-2 constant region; or (c) an equine heavy chain constant region selected from an IgG-1, IgG-2, IgG-3, IgG-4, IgG-5, IgG-6, and IgG-7 constant region.

Embodiment 39. The antibody of any one of embodiments 1 to 38, wherein the antibody is an antibody fragment, such as an Fv, scFv, Fab, Fab′, F(ab′)2, or Fab′-SH fragment.

Embodiment 40. The antibody of any one of embodiments 1 to 39, wherein the antibody is bi-specific, wherein the antibody binds to IL17A and one or more antigens selected from other members of IL17, IL31, TNFα, CD20, CD19, CD25, IL4, IL13, IL23, IgE, CD11α, IL6R, α4-Intergrin, IL12, IL1β, or BlyS.

Embodiment 41. An isolated nucleic acid encoding the antibody of any one of embodiments 1 to 40.

Embodiment 42. A host cell comprising the nucleic acid of embodiment 41.

Embodiment 43. A method of producing an antibody comprising culturing the host cell of embodiment 42 and isolating the antibody.

Embodiment 44. A pharmaceutical composition comprising the antibody of any one of embodiments 1 to 40 and a pharmaceutically acceptable carrier.

Embodiment 45. A method of treating a companion animal species having an IL17A-induced condition, the method comprising administering to the companion animal species a therapeutically effective amount of the antibody of any one of embodiments 1 to 40 or the pharmaceutical composition of embodiment 45.

Embodiment 46. The method of embodiment 45, wherein the companion animal species is canine, feline, or equine.

Embodiment 47. The method of embodiment 45 or 46, wherein the IL17A-induced condition is plaque psoriasis, psoriatic arthritis, rheumatoid arthritis, airway inflammation, asthma, osteoarthritis, inflammatory bowel disorder, Crohn's disease, ankylosing spondylitis, atopic dermatitis, degenerative myelopathy, multiple sclerosis, or uveitis.

Embodiment 48. The method of any one of embodiments 45 to 47, wherein the antibody or the pharmaceutical composition is administered parenterally.

Embodiment 49. The method of any one of embodiments 45 to 48, wherein the antibody or the pharmaceutical composition is administered by an intramuscular route, an intraperitoneal route, an intracerebrospinal route, a subcutaneous route, an intra-arterial route, an intrasynovial route, an intrathecal route, or an inhalation route.

Embodiment 50. The method of any one of embodiments 45 to 49, wherein the method comprises administering in combination with the antibody or the pharmaceutical composition a NFκB inhibitor, a MAPK inhibitor, and/or a C/EBP inhibitor.

Embodiment 51. The method of any one of embodiments 45 to 50, wherein the method comprises administering in combination with the antibody or the pharmaceutical composition one or more antibodies selected from an anti-IL17A antibody, an anti-TNFα antibody, an anti-CD20 antibody, an anti-IL31 antibody, an anti-CD19 antibody, an anti-CD25 antibody, an anti-IL4 antibody, an anti-IL13 antibody, an anti-IL23 antibody, an anti-IgE antibody, an anti-CD11a antibody, anti-IL6R antibody, anti-α4-Intergrin antibody, an anti-IL12 antibody, an anti-IL13 antibody, and an anti-BlyS antibody.

Embodiment 52. A method of reducing IL17A signaling function in a cell, the method comprising exposing to the cell the antibody of any one of embodiments 1 to 40 or the pharmaceutical composition of embodiment 45 under conditions permissive for binding of the antibody to extracellular IL17A, thereby reducing binding to IL17A receptor and/or reducing IL17A signaling function by the cell.

Embodiment 53. The method of embodiment 52, wherein the cell is exposed to the antibody or the pharmaceutical composition ex vivo.

Embodiment 54. The method of embodiment 52, wherein the cell is exposed to the antibody or the pharmaceutical composition in vivo.

Embodiment 55. The method of embodiment 42, 43, or 44, wherein the cell is a canine cell, a feline cell, or an equine cell.

Embodiment 56. A method for detecting IL17A in a sample from a companion animal species comprising contacting the sample with the antibody of any one of embodiments 1 to 40 or the pharmaceutical composition of embodiment 45 under conditions permissive for binding of the antibody to IL17A, and detecting whether a complex is formed between the antibody and IL17A in the sample.

Embodiment 57. The method of embodiment 56, wherein the sample is a biological sample obtained from a canine, a feline, or an equine.

Embodiment 58. A polypeptide comprising an extracellular domain of an IL17A receptor (IL17Ra) polypeptide comprising the amino acid sequence of SEQ ID NO: 94, SEQ ID NO: 97, SEQ ID NO: 98, or SEQ ID NO: 99.

Embodiment 59. The polypeptide of embodiment 58 comprising the amino acid sequence of SEQ ID NO: 33.

Embodiment 60. The polypeptide of embodiment 58 or embodiment 59, wherein the IL17Ra polypeptide comprises an IgG Fc.

Embodiment 61. The polypeptide of any one of embodiments 58 to 60, wherein the IgG Fc is
a. a human IgG1 Fc, IgG2 Fc, IgG3 Fc, or IgG4 Fc;
b. a canine IgG-A Fc, IgG-B Fc, IgG-C Fc, or IgG-D Fc;
c. a feline IgG1a Fc, IgG1 b Fc, or IgG2 Fc; or
d. an equine IgG1 Fc, IgG2 Fc, IgG3 Fc, IgG4 Fc, IgG5 Fc, IgG6 Fc, or IgG7 Fc.

Embodiment 62. The polypeptide of any one of embodiments 58 to 61, wherein the IL17Ra polypeptide comprises the amino acid sequence of SEQ ID NO: 40, SEQ ID NO: 41, SEQ ID NO: 42, SEQ ID NO: 43, or SEQ ID NO: 44.

Embodiment 63. An isolated nucleic acid encoding the polypeptide of embodiment 58 or embodiment 59.

Embodiment 64. A host cell comprising the nucleic acid of embodiment 60.

Embodiment 65. A method of producing a polypeptide comprising culturing the host cell of embodiment and isolating the polypeptide.

Embodiment 66. A pharmaceutical composition comprising the polypeptide of embodiment 58 or embodiment 59 and a pharmaceutically acceptable carrier.

Embodiment 67. A method of treating a companion animal species having an IL17A-induced condition, the method comprising administering to the subject a therapeutically effective amount of the polypeptide of embodiment 58 or embodiment 59, or the pharmaceutical composition of embodiment 63.

Embodiment 68. The method of embodiment 64, wherein the polypeptide or pharmaceutical composition is administered parenterally.

Embodiment 69. The method of embodiment 64 or embodiment 65, wherein the polypeptide or pharmaceutical composition is administered by an intramuscular route, an intraperitoneal route, an intracerebrospinal route, a subcutaneous route, an intra-arterial route, an intrasynovial route, an intrathecal route, or an inhalation route.

Embodiment 70. The method of any one of embodiments 64 to 66, wherein the companion animal species is feline, canine, or equine.

Embodiment 71. The method of any one of embodiments 64 to 67, wherein the IL17A-induced condition is plaque psoriasis, psoriatic arthritis, rheumatoid arthritis, airway inflammation, asthma, osteoarthritis, inflammation bowel disorder, Crohn's disease, ankylosing spondylitis, atopic dermatitis, degenerative myelopathy, multiple sclerosis, or uveitis.

Embodiment 72. A polypeptide comprising an IgG Fc variant polypeptide comprising at least one amino acid substitution relative to a IgG Fc wild-type polypeptide derived from a companion animal species, wherein the IgG Fc variant polypeptide has reduced binding to C1q and/or CD16 relative to the IgG Fc wild-type polypeptide.

Embodiment 73. The polypeptide of embodiment 72, wherein the IgG Fc variant polypeptide binds to C1q and/or CD16 with a dissociation constant (Kd) of less than 5×10-6 M, less than 1×10-6 M, less than 5×10-7 M, less than 1×10-7 M, less than 5×10-8 M, less than 1×10-8 M, less than 5×10-9 M, less than 1×10-9 M, less than 5×10-10 M, less than 1×10-10 M, less than 5×10-11 M, less than 1×10-11 M, less than 5×10-12 M, or less than 1×10-12 M, as measured by biolayer interferometry.

Embodiment 74. The polypeptide of embodiment 72 or embodiment 73, wherein the companion animal species is canine, feline, or equine.

Embodiment 75. The polypeptide of any one of embodiments 72 to 74, wherein the wild-type IgG Fc polypeptide is a canine IgG-B Fc or canine IgG-C Fc.

Embodiment 76. The polypeptide of any one of embodiments 72 to 75, wherein the IgG Fc variant polypeptide comprises an amino acid substitution at a position corresponding to position 110 of SEQ ID NO: 45 or at a position corresponding to position 108 of SEQ ID NO: 46.

Embodiment 77. The polypeptide of any one of embodiments 72 to 76, wherein the IgG Fc variant polypeptide comprises an amino acid substitution at a position corresponding to position 55 of SEQ ID NO: 45 or at a position corresponding to position 43 of SEQ ID NO: 46.

Embodiment 78. The polypeptide of any one of embodiments 72 to 77, wherein the IgG Fc variant polypeptide comprises an amino acid substitution at a position corresponding to position 114 of SEQ ID NO: 45 or at a position corresponding to position 112 of SEQ ID NO: 46.

Embodiment 79. The polypeptide of any one of embodiments 72 to 78, wherein the IgG Fc variant polypeptide comprises an amino acid substitution at a position corresponding to position 115 at SEQ ID NO: 45 or at a position corresponding to position 113 of SEQ ID NO: 46.

Embodiment 80. The polypeptide of any one of embodiments 72 to 79, wherein the IgG Fc variant polypeptide comprises an amino acid substitution at position 110 of SEQ ID NO: 45 or at position 108 of SEQ ID NO: 46.

Embodiment 81. The polypeptide of any one of embodiments 72 to 80, wherein the IgG Fc variant polypeptide comprises an amino acid substitution at position 55 of SEQ ID NO: 45 or at position 43 of SEQ ID NO: 46.

Embodiment 82. The polypeptide of any one of embodiments 72 to 81, wherein the IgG Fc variant polypeptide comprises an amino acid substitution at position 114 of SEQ ID NO: 45 or at position 112 of SEQ ID NO: 46.

Embodiment 83. The polypeptide of any one of embodiments 72 to 82, wherein the IgG Fc variant polypeptide comprises an amino acid substitution at position 115 at SEQ ID NO: 45 or at position 113 of SEQ ID NO: 46.

Embodiment 84. The polypeptide of any one of embodiments 72 to 83, wherein the IgG Fc variant polypeptide comprises an arginine at position 110 of SEQ ID NO: 45 or at position 108 of SEQ ID NO: 46.

Embodiment 85. The polypeptide of any one of embodiments 72 to 84, wherein the IgG Fc variant polypeptide comprises a glycine at position 55 of SEQ ID NO: 45 or at position 43 of SEQ ID NO: 46.

Embodiment 86. The polypeptide of any one of embodiments 72 to 85, wherein the IgG Fc variant polypeptide comprises an isoleucine at position 114 of SEQ ID NO: 45 or at position 112 of SEQ ID NO: 46.

Embodiment 87. The polypeptide of any one of embodiments 72 to 86, wherein the IgG Fc variant polypeptide comprises a glycine at position 115 at SEQ ID NO: 45 or at position 113 of SEQ ID NO: 46.

Embodiment 88. The polypeptide of any one of embodiments 72 to 87 comprising the amino acid sequence of SEQ ID NO: 47, SEQ ID NO: 48, SEQ ID NO: 49, or SEQ ID NO: 50.

Embodiment 89. The polypeptide of any one of embodiments 72 to 88, wherein the at least one amino acid substitution comprises an amino acid substitution with an amino acid derivative.

Embodiment 90. The polypeptide of any one of embodiments 72 to 89, wherein the polypeptide is an antibody, an antibody fragment, or a fusion polypeptide.

Embodiment 91. The polypeptide of any one of embodiments 72 to 90, wherein the polypeptide comprises the amino acid sequence of SEQ ID NO: 96.

Embodiment 92. The polypeptide of any one of embodiments 72 to 90, wherein the polypeptide comprises the antibody of any one of embodiments 1 to 40 or the polypeptide of any one of embodiments 58 to 62.

Embodiment 93. An isolated nucleic acid encoding the polypeptide of any one of embodiments 72 to 92.

Embodiment 94. A host cell comprising the nucleic acid of embodiment 93.

Embodiment 95. A method of producing a polypeptide comprising culturing the host cell of embodiment 94 and isolating the polypeptide.

Embodiment 96. A pharmaceutical composition comprising the polypeptide of any one of embodiments 72 to 92 and a pharmaceutically acceptable carrier.

Embodiment 97. A method of delivering a polypeptide to a subject comprising administering the polypeptide of any one of embodiments 72 to 92 or the pharmaceutical composition of embodiment 96 parenterally.

Embodiment 98. The method of embodiment 97 comprising administering the polypeptide or the pharmaceutical composition by an intramuscular route, an intraperitoneal route, an intracerebrospinal route, a subcutaneous route, an intra-arterial route, an intrasynovial route, an intrathecal route, or an inhalation route.

Embodiment 99. The method of embodiment 97 or embodiment 98, wherein the species is human.

Embodiment 100. The method of embodiment 97 or embodiment 98, wherein the species is a companion animal species.

Embodiment 101. The method of embodiment 100, wherein the companion animal species is canine, equine, or feline.

### BRIEF DESCRIPTION OF THE DRAWINGS

FIG. 1 shows a canine IL17A binding ELISA of Clone A, B, C, D, and E mouse monoclonal antibodies.

FIGS. 2A-2B show the amino acid sequence alignment of variable light chain (A) and variable heavy chain (B) sequences of Clone D, C, A, and E mouse monoclonal antibodies.

FIGS. 3A-3B show a canine IL17A binding analysis using various concentrations of Clone C antibody (A) and caninized Clone C antibody (B).

FIG. 4 shows an H1080 cell-based canine IL17A neutralization assay of caninized Clone C antibody.

FIG. 5 shows Western blots of canine IL17A-hFc after separation by SDS-PAGE in the presence of DTT (+) or absence of DTT (−), transfer to PVDF membrane, and being probed with Clone A, C, D, or E antibody followed by goat anti-mouse IgG-HRP. Immunoreactive positive signals were only observed under non-reducing conditions.

FIGS. 6A-6D show Western blots of IL17A proteins (0.6 μg) probed with Clone A, Clone C, Clone D, or Clone E antibodies followed by goat anti-mouse IgG-HRP. Lane 1: Protein MW marker; Lane 2: canine IL17A-hFc; Lane 3: Feline IL17A-polyHis; Lane 4: Equine IL17A-polyHis; Lane 5: Canine IL17F.

FIGS. 7A-7B show Western blots of canine IL17A-hFc-polyHis mutant proteins after separation by SDS-PAGE in the presence (+) or absence (−) of DDT, transfer to PVDF membrane, and being probed with either Clone C antibody (A) or anti-human IgG Fc antibody (B). Lane 1: Mutant 5 (+DTT); Lane 2: Mutant 5 (−DTT); Lane 3: Mutant 1 (+DTT); Lane 4: Mutant 1 (−DTT); Lane 5: Mutant 2 (+DTT); Lane 6: Mutant 2 (−DTT); Lane 7: Mutant 3 (+DTT); Lane 8: Mutant 3 (−DTT); Lane 9: Mutant 4 (+DTT); Lane 10: Mutant 4 (−DTT).

### DESCRIPTION OF THE SEQUENCES

Table 1 provides a listing of certain sequences referenced herein.

Description of the Sequences

| SEQ ID | | |
|---|---|---|
| NO: | SEQUENCE | DESCRIPTION |
| 1 | GFTFSSYGMS | Variable heavy chain CDR- |
| | | H1 amino acid sequence of |
| | | mouse antibody clone C |
| 2 | IINSNGGSTYYPDSVKG | Variable heavy chain CDR- |
| | | H2 amino acid sequence of |
| | | mouse antibody clone C |
| 3 | CHYDYERVFDY | Variable heavy chain CDR- |
| | | H3 amino acid sequence of |
| | | mouse antibody clone C |
| 4 | EVQLVESGGGLVQPGGSLKLSCAAS | Variable region heavy chain |
| | | framework HC-FR1 amino |
| | | acid sequence of mouse |
| | | antibody clone C |
| 5 | WVRQTPDKRLELVA | Variable region heavy chain |
| | | framework HC-FR2 amino |
| | | acid sequence of mouse |
| | | antibody clone C |
| 6 | RFTISRDNDKNSLYLQMSSLKSEDTAMYYCVR | Variable region heavy chain |
| | | framework HC-FR3 amino |
| | | acid sequence of mouse |
| | | antibody clone C |
| 7 | WGQGTTLTVSS | Variable region heavy chain |
| | | framework HC-FR4 amino |
| | | acid sequence of mouse |
| | | antibody clone C |
| 8 | KANDHINNWLA | Variable light chain CDR- |
| | | L1 amino acid sequence of |
| | | mouse antibody clone C |
| 9 | GSTSLET | Variable light chain CDR- |
| | | L2 amino acid sequence of |
| | | mouse antibody clone C |
| 108 | GSTSLES | Variable light chain CDR- |
| | | L2 v2 amino acid sequence |
| 10 | QQYWSTPFT | Variable light chain CDR- |
| | | L3 amino acid sequence of |
| | | mouse antibody clone C |
| 11 | DIQMTQSSSYLSVSLGGRVTITC | Variable region light chain |
| | | framework LC-FR1 amino |
| | | acid sequence of mouse |
| | | antibody clone Cl |
| 12 | WYQQKPGNAPRLLIS | Variable region light chain |
| | | framework LC-FR2 amino |
| | | acid sequence of mouse |
| | | antibody clone C |
| 13 | GVPSRFSGSGSGKDYTLSITSLQTEDVATYYC | Variable region light chain |
| | | framework LC-FR3 amino |
| | | acid sequence of mouse |
| | | antibody clone C |
| 14 | FGSGTKLEIK | Variable region light chain |
| | | framework LC-FR4 amino |
| | | acid sequence of mouse |
| | | antibody clone C |
| 15 | EVQLVESGGGLVKPGGSLRLSCAASGFTFSSYGMSWV | Caninized variable heavy |
| | RQAPGKRLELVAIINSNGGSTYYPDSVKGRFTFSLDT | chain amino acid sequence |
| | SKNTLYLQMNSLRAEDTAMYYCVRCHYDYERVFDYWG | of mouse antibody clone C |
| | QGTLVTVSS | |
| 16 | DIQMTQSPASVSGSLGDKVSITCKANDHINNWLAWYQ | Caninized variable light |
| | QLPGNAPRLLISGSTSLESGVPDRFSGSKSGSSFTLT | chain amino acid sequence |
| | ISGLQPEDFATYYCQQYWSTPFTFGSGTKVEIK | of mouse antibody clone C |
| 17 | EVQLVESGGGLVKPGGSLRLSCAASGFTFSSYGMSWV | Caninized heavy chain |
| | RQAPGKRLELVAIINSNGGSTYYPDSVKGRFTFSLDT | sequence from mouse |
| | SKNTLYLQMNSLRAEDTAMYYCVRCHYDYERVFDYWG | antibody clone C and canine |
| | QGTLVTVSSASTTAPSVFPLAPSCGSTSGSTVALACL | IgG-A |
| | VSGYFPEPVTVSWNSGSLTSGVHTFPSVLQSSGLHSL | |
| | SSMVTVPSSRWPSETFTCNVVHPASNTKVDKPVFNEC | |
| | RCTDTPCPVPEPLGGPSVLIFPPKPKDILRITRTPEV | |
| | TCVVLDLGREDPEVQISWFVDGKEVHTAKTQSREQQF | |
| | NGTYRVVSVLPIEHQDWLTGKEFKCRVNHIDLPSPIE | |
| | RTISKARGRAHKPSVYVLPPSPKELSSSDTVSITCLI | |
| | KDFYPPDIDVEWQSNGQQEPERKHRMTPPQLDEDGSY | |
| | FLYSKLSVDKSRWQQGDPFTCAVMHETLQNHYTDLSL | |
| | SHSPGK | |
| 18 | EVQLVESGGGLVKPGGSLRLSCAASGFTFSSYGMSWV | Caninized heavy chain |
| | RQAPGKRLELVAIINSNGGSTYYPDSVKGRFTFSLDT | sequence from mouse |
| | SKNTLYLQMNSLRAEDTAMYYCVRCHYDYERVFDYWG | antibody clone C and canine |
| | QGTLVTVSSASTTAPSVFPLAPSCGSTSGSTVALACL | IgG-B |
| | VSGYFPEPVTVSWNSGSLTSGVHTFPSVLQSSGLYSL | |
| | SSMVTVPSSRWPSETFTCNVAHPASKTKVDKPVPKRE | |
| | NGRVPRPPDCPKCPAPEMLGGPSVFIFPPKPKDTLLI | |
| | ARTPEVTCVVVDLDPEDPEVQISWFVDGKQMQTAKTQ | |
| | PREEQFNGTYRVVSVLPIGHQDWLKGKQFTCKVNNKA | |
| | LPSPIERTISKARGQAHQPSVYVLPPSREELSKNTVS | |
| | LTCLIKDFFPPDIDVEWQSNGQQEPESKYRTTPPQLD | |
| | EDGSYFLYSKLSVDKSRWQRGDTFICAVMHEALHNHY | |
| | TQESLSHSPGK | |
| 19 | EVQLVESGGGLVKPGGSLRLSCAASGFTFSSYGMSWV | Caninized heavy chain |
| | RQAPGKRLELVAIINSNGGSTYYPDSVKGRFTFSLDT | sequence from mouse |
| | SKNTLYLQMNSLRAEDTAMYYCVRCHYDYERVFDYWG | antibody clone C and canine |
| | QGTLVTVSSASTTAPSVFPLAPSCGSQSGSTVALACL | IgG-C |
| | VSGYIPEPVTVSWNSVSLTSGVHTFPSVLQSSGLYSL | |
| | SSMVTVPSSRWPSETFTCNVAHPATNTKVDKPVAKEC | |
| | ECKCNCNNCPCPGCGLLGGPSVFIFPPKPKDILVTAR | |
| | TPTVTCVVVDLDPENPEVQISWFVDSKQVQTANTQPR | |
| | EEQSNGTYRVVSVLPIGHQDWLSGKQFKCKVNNKALP | |
| | SPIEEIISKTPGQAHQPNVYVLPPSRDEMSKNTVTLT | |
| | CLVKDFFPPEIDVEWQSNGQQEPESKYRMTPPQLDED | |
| | GSYFLYSKLSVDKSRWQRGDTFICAVMHEALHNHYTQ | |
| | ISLSHSPGK | |
| 20 | EVQLVESGGGLVKPGGSLRLSCAASGFTFSSYGMSWV | Caninized heavy chain |
| | RQAPGKRLELVAIINSNGGSTYYPDSVKGRFTFSLDT | sequence from mouse |
| | SKNTLYLQMNSLRAEDTAMYYCVRCHYDYERVFDYWG | antibody clone Clone C and |
| | QGTLVTVSSASTTAPSVFPLAPSCGSTSGSTVALACL | canine IgG-D |
| | VSGYFPEPVTVSWNSGSLTSGVHTFPSVLQSSGLYSL | |
| | SSTVTVPSSRWPSETFTCNVVHPASNTKVDKPVPKES | |
| | TCKCISPCPVPESLGGPSVFIFPPKPKDILRITRTPE | |
| | ITCVVLDLGREDPEVQISWFVDGKEVHTAKTQPREQQ | |
| | FNSTYRVVSVLPIEHQDWLTGKEFKCRVNHIGLPSPI | |
| | ERTISKARGQAHQPSVYVLPPSPKELSSSDTVTLICL | |
| | IKDFFPPEIDVEWQSNGQPEPESKYHTTAPQLDEDGS | |
| | YFLYSKLSVDKSRWQQGDIFTCAVMHEALQNHYTDLS | |
| | LSHSPGK | |
| 21 | DIQMTQSPASVSGSLGDKVSITCKANDHINNWLAWYQ | Caninized light chain |
| | QLPGNAPRLLISGSTSLESGVPDRFSGSKSGSSFTLT | sequence from mouse |
| | ISGLQPEDFATYYCQQYWSTPFTFGSGTKVEIKRNDA | antibody clone C and canine |
| | QPAVYLFQPSPDQLHTGSASVVCLLNSFYPKDINVKW | light chain constant region |
| | KVDGVIQDTGIQESVTEQDKDSTYSLSSTLTMSSTEY | |
| | LSHELYSCEITHKSLPSTLIKSFQRSECQRVD | |
| 22 | AGIAFPQNPGCRNTEDKNFPQHVKVNLNILNRNTNSR | Mature canine IL17A amino |
| | RPSDYYNRSTSPWNLHRNEDPERYPSVIWEAKCRHLG | acid sequence |
| | CVNNEGNINYHMNSVPIQQEILVLRRESQHCPHSFRL | |
| | EKMLVAVGCTCVTPIVRHVA | |
| 23 | RHLGCVNNEGNI | Canine IL17A epitope, |
| | | minimal sequence |
| 24 | DIQMTQSSSYLSVSLGGRVTITCKANDHINNWLAWYQ | Variable light chain amino |
| | QKPGNAPRLLISGSTSLETGVPSRFSGSGSGKDYTLS | acid sequence of mouse |
| | ITSLQTEDVATYYCQQYWSTPFTFGSGTKLEIK | antibody clone C |
| 25 | EVQLVESGGGLVQPGGSLKLSCAASGFTFSSYGMSWV | Variable heavy chain amino |
| | RQTPDKRLELVAIINSNGGSTYYPDSVKGRFTISRDN | acid sequence of mouse |
| | DKNSLYLQMSSLKSEDTAMYYCVRCHYDYERVFDYWG | antibody clone C |
| | QGTTLTVSS | |
| 26 | DIQMTQSSSYLSVSLGGRVTITCKANDHINNWLAWYQ | Chimeric variable light |
| | QKPGNAPRLLISGSTSLETGVPSRFSGSGSGKDYTLS | chain of mouse antibody |
| | ITSLQTEDVATYYCQQYWSTPFTFGSGTKLEIKRNDA | clone C and canine light |
| | QPAVYLFQPSPDQLHTGSASVVCLLNSFYPKDINVKW | chain constant region |
| | KVDGVIQDTGIQESVTEQDKDSTYSLSSTLTMSSTEY | |
| | LSHELYSCEITHKSLPSTLIKSFQRSECQRVD | |
| 27 | EVQLVESGGGLVQPGGSLKLSCAASGFTFSSYGMSWV | Chimeric variable heavy |
| | RQTPDKRLELVAIINSNGGSTYYPDSVKGRFTISRDN | chain of mouse antibody |
| | DKNSLYLQMSSLKSEDTAMYYCVRCHYDYERVFDYWG | clone C and canine IgG-B |
| | QGTTLTVSSASTTAPSVFPLAPSCGSTSGSTVALACL | |
| | VSGYFPEPVTVSWNSGSLTSGVHTFPSVLQSSGLYSL | |
| | SSMVTVPSSRWPSETFTCNVAHPASKTKVDKPVPKRE | |
| | NGRVPRPPDCPKCPAPEMLGGPSVFIFPPKPKDTLLI | |
| | ARTPEVTCVVVDLDPEDPEVQISWFVDGKQMQTAKTQ | |
| | PREEQFNGTYRVVSVLPIGHQDWLKGKQFTCKVNNKA | |
| | LPSPIERTISKARGQAHQPSVYVLPPSREELSKNTVS | |
| | LTCLIKDFFPPDIDVEWQSNGQQEPESKYRTTPPQLD | |
| | EDGSYFLYSKLSVDKSRWQRGDTFICAVMHEALHNHY | |
| | TQESLSHSPGK | |
| 28 | MTLVTTSSMFQSLLLLLSLVAIIKAGIAFPQNPGCRN | Canine IL17A precursor |
| | TEDKNFPQHVKVNLNILNRNTNSRRPSDYYNRSTSPW | amino acid sequence |
| | NLHRNEDPERYPSVIWEAKCRHLGCVNNEGNINYHMN | |
| | SVPIQQEILVLRRESQHCPHSFRLEKMLVAVGCTCVT | |
| | PIVRHVA | |
| 29 | MAPLRTSSVSLLLLLSLVAIVKAGIVIPQNPECPNTG | Equine IL17A precursor |
| | DKNFPQNVKINLNVLNRKTNSRRASDYHNRSTSPWNL | amino acid sequence |
| | HRNEDPERYPSVIWEAKCRHLGCVNAEGKVDFHMNSV | |
| | PIQQEILVLRRESQNCPHSFQLEKMLVAVGCTCVTPI | |
| | VRHMG | |
| 30 | MAPGTTSSMFPSLLLLLCLMAIVRTGIAFPQNPGCPT | Feline IL17A precursor |
| | TEDKNFPQHVKVNVNILNGNKSSRRPLDYYRRSTSPW | amino acid sequence |
| | SLHRNEDPERYPSVIWEAKCLHWGCVNTEGKEDHHMN | |
| | SVPIQQEILVLRRESRHCPHSFRLEKMLVTVGCTCVT | |
| | PIVRHVV | |
| 31 | MAILRNIAMVKSLLLLVLGLTLLSEVAARKHLKAGET | Canine IL17F precursor |
| | ALCPPLEDNSVRVDIRILRQNRGISISNDFQNRSSSP | amino acid sequence |
| | WDYNITRDPHRFPSEIAEAQCRHSGCINAEGQEDSSM | |
| | NSVPIQQEFLVLRREPQGCSRSFRLEKVLVTVGCTCV | |
| | TPIVRYVRA | |
| 32 | MGRLGEGLNCTVKNSTCLDDSWIHPRNLTPSSPKDVQ | Canine IL17A receptor |
| | VHLDFAQTQHGDLLPIIGIRWTLQTDASILFLEGAEL | (IL17Ra) amino acid |
| | SVLQLNTNERVCVKFEFLSKLKHHHKRWHFTFSHFVV | sequence |
| | EPGQEYEVTVHHLPKPIPDGDPNHQSKNFLVPGCEDP | |
| | RMRMTTPCVSSGSLWDPNITAEALEAHQLQVHFTLWN | |
| | ESAQYQILLTSFPHTENRSCFHRVLMVPEPTLKEHHQ | |
| | RANIMLTGSSSNWCCRHQVQIQPFFSSCLNDCLRHSV | |
| | TVPCPEIPDAPVSIADYIPLWAYGFITGIAILLVGSV | |
| | ILLIVCMAWRLPGSHCEKYGNDSKYTDIQPKTSLTPP | |
| | PLKPRKVWIVYSADHPLYVDVVLKFAQFLLTVCGTEV | |
| | ALDLLEEQVISEVGVMTWVGRQKQEMVETNSKIIILC | |
| | SRGTRAKWQAILGWEEPAVQLRCDRWKPSGDLFTAAM | |
| | NMILPDFKKPACFGTYIICYFRDISSESDIPDLFNIT | |
| | SRYPLMDKFEEVYFRIQDLEMFEPGRMHRVGELTGEN | |
| | YLQSPSGWQLKEAVERFREWQVRCPDWFERENLGSAD | |
| | DQDLPSLDEEVFEEPLLPPGRGIVKQKPLVHEPAPEG | |
| | CLVIDLLVGEEGRGPSRLEPQLQPQGELMAQTLQTVV | |
| | FPVKEVPSAQAVEPVPHTVESSTAGRLAVVEGDEACP | |
| | LLEGCGPWRNSVLCLPMDSEEPPLCRTPMASPSYLPE | |
| | DVREQLEGLMFSLLEQSLSCQAQEGWDRAAVALKDFR | |
| | TPYEEEQRQSVQSDQGYISRSSPQPPEGLMEMEEEEA | |
| | EQDLGKSAKQLSPEDLESLRSLQRQLFFQELQTNSGW | |
| | DSVELEVP | |
| 33 | MGRLGEGLNCTVKNSTCLDDSWIHPRNLTPSSPKDVQ | Canine IL17A receptor |
| | VHLDFAQTQHGDLLPIIGIRWTLQTDASILFLEGAEL | (IL17Ra) ECD (binding |
| | SVLQLNTNERVCVKFEFLSKLKHHHKRWHFTFSHFVV | domain fragment) amino |
| | EPGQEYEVTVHHLPKPIPDGDPNHQSKNFLVPGCEDP | acid sequence |
| | RMRMTTPCVSSGSLWDPNITAEALEAHQLQVHFTLWN | |
| | ESAQYQILLTSFPHTENRSCFHRVLMVPEPTLKEHHQ | |
| | RANIMLTGSSSNWCCRHQVQIQPFFSSCLNDCLRHSV | |
| | TVPCPEIPDAPVSIADYIPL | |
| 34 | DVVMTQTPLSLPVSLGDQASISCRSSQSLVHSNGNTY | Variable light chain amino |
| | LHWYLQRPGQSPNLLIYKVSNRFSGVPDRFSGSGSGT | acid sequence of mouse |
| | DFTLKISRVEAEDLGVYFCSQSTHVPFTFGSGTKLEI | antibody clone A |
| | K | |
| 35 | QVQLKESGPGLVAPSQSLSITCTISGFSLTSNGVHWV | Variable heavy chain amino |
| | RQSPGKDLEWLVVIWSDGTTTYNSDFKSRLSISKDNS | acid sequence of mouse |
| | KSQVFLKMNSLQTDDTAMYYCARHYDWGYYYAMDYWG | antibody clone A |
| | QGTSVTVSS | |
| 36 | DIVLTQSPASLAVSLGQRATISYRASKSVSTSGYSY | Variable light chain amino |
| | MHWNQQKPGQPPRLLIYLVSNLESGVPARFSGSGSG | acid sequence of mouse |
| | TDFTLNIHPVEEEDAATYYCQHIRELYTFGGGTKLE | antibody clone D |
| | IK | |
| 37 | EVQLQQSGPELVKTGASVKISCKASGYSFTYYYMHWV | Variable heavy chain amino |
| | KQSHGKSLEWIGYISCFNGDTNYNQEFKDKATFTADT | acid sequence of mouse |
| | SSSTAYMQFNSLTSEDSAVYYCARGLSTLITEGWFAY | antibody clone D |
| | WGQGTLVTVSA | |
| 38 | DVVMTQTPLSLPVSLGDQASISCRSSQSLVHSNGNTY | Variable light chain amino |
| | FHWYLQKPGQSPKLLIYKVSNRFSGVPDRFSGSGSGT | acid sequence of mouse |
| | DFTLKISRVEAEDLGVYFCSQSTHAPFTFGSGTKLEI | antibody clone E |
| | K | |
| 39 | QVQLKESGPGLVAPSQSLSITCTISGFSLTSNGVHWV | Variable heavy chain amino |
| | RQPPGKGLEWLVVIWSDGTTTYNSALKSRLSISKDNS | acid sequence of mouse |
| | KSQVFLKMNSLQTDDTAMYYCARHYDRGYYYAMDYWG | antibody clone E |
| | QGTSVTVSS | |
| 40 | SLRLLDHRALVCSQPGLNCTVKNSTCLDDSWIHPRNL | Human IL17Ra ECD-IgG4-Fc |
| | TPSSPKDLQIQLHFAHTQQGDLFPVAHIEWTLQTDAS | |
| | ILYLEGAELSVLQLNTNERLCVRFEFLSKLRHHHRRW | |
| | RFTFSHFVVDPDQEYEVTVHHLPKPIPDGDPNHQSKN | |
| | FLVPDCEHARMKVTTPCMSSGSLWDPDITVETLEAHQ | |
| | LRVSFTLWNESTHYQILLTSFPHMENHSCFEHMHHIP | |
| | APRPEEFHQRSDVTLTLRNLKGCCRHQVQIQPFFSSC | |
| | LNDCLRHSATVSCPEMPDTPEPIPDGSESKYGPPCPP | |
| | CPAPEAAGGPSVFLFPPKPKDTLMISRTPEVTCVVVD | |
| | VSQEDPEVQFNWYVDGVEVHNAKTKPREEQFNSTYRV | |
| | VSVLTVLHQDWLNGKEYKCKVSNKGLPSSIEKTISKA | |
| | KGQPREPQVYTLPPSQEEMTKNQVSLTCLVKGFYPSD | |
| | IAVEWESNGQPENNYKTTPPVLDSDGSFFLYSRLTVD | |
| | KSRWQEGNVFSCSVMHEALHNHYTQKSLSLSLG | |
| 41 | MGRLGEGLNCTVKNSTCLDDSWIHPRNLTPSSPKDVQ | Canine IL17Ra ECD-canine |
| | VHLDFAQTQHGDLLPIIGIRWTLQTDASILFLEGAEL | IgG-B-Fc |
| | SVLQLNTNERVCVKFEFLSKLKHHHKRWHFTFSHFVV | |
| | EPGQEYEVTVHHLPKPIPDGDPNHQSKNFLVPGCEDP | |
| | RMRMTTPCVSSGSLWDPNITAEALEAHQLQVHFTLWN | |
| | ESAQYQILLTSFPHTENRSCFHRVLMVPEPTLKEHHQ | |
| | RANIMLTGSSSNWCCRHQVQIQPFFSSCLNDCLRHSV | |
| | TVPCPEIPDAPVSIADYIGSPKRENGRVPRPPDCPKC | |
| | PAPEMLGGPSVFIFPPKPKDILLIARTPEVICVVVDL | |
| | DPEDPEVQISWFVDGKQMQTAKTQPREEQFNGTYRVV | |
| | SVLPIGHQDWLKGKQFTCKVNNKALPSPIERTISKAR | |
| | GQAHQPSVYVLPPSREELSKNTVSLTCLIKDFFPPDI | |
| | DVEWQSNGQQEPESKYRTTPPQLDEDGSYFLYSKLSV | |
| | DKSRWQRGDTFICAVMHEALHNHYTQESLSHSPGK | |
| 42 | MDMRVPAQLLGLLLLWLRGARCMGRLGEGLNCTVKNS | canine IL17Ra ECD-huFc- |
| | TCLDDSWIHPRNLTPSSPKDVQVHLDFAQTQHGDLLP | polyHis |
| | IIGIRWTLQTDASILFLEGAELSVLQLNTNERVCVKF | |
| | EFLSKLKHHHKRWHFTFSHFVVEPGQEYEVTVHHLPK | |
| | PIPDGDPNHQSKNFLVPGCEDPRMRMTTPCVSSGSLW | |
| | DPNITAEALEAHQLQVHFTLWNESAQYQILLTSFPHT | |
| | ENRSCFHRVLMVPEPTLKEHHQRANIMLTGSSSNWCC | |
| | RHQVQIQPFFSSCLNDCLRHSVTVPCPEIPDAPVSIA | |
| | DYIGSENLYFQGPKSCDKTHTCPPCPAPELLGGPSVF | |
| | LFPPKPKDTLMISRTPEVTCVVVDVSHEDPEVKFNWY | |
| | VDGVEVHNAKTKPREEQYNSTYRVVSVLTVLHQDWLN | |
| | GKEYKCKVSNKALPAPIEKTISKAKGQPREPQVYTLP | |
| | PSRDELTKNQVSLTCLVKGFYPSDIAVEWESNGQPEN | |
| | NYKTTPPVLDSDGSFFLYSKLTVDKSRWQQGNVFSCS | |
| | VMHEALHNHYTQKSLSLSPGKHHHHHH | |
| 43 | SPRLLDYPAPVCSQQGLNCVVKNSTCLDDSWIHLRNL | Feline IL17Ra ECD-feline |
| | TPSSPKDVQVHLDFVQTQHGDLLPVAGIRWTLQTDAS | IgG-2-Fc |
| | ILYLEGAELSVLQLNTNERLCVKFEFLTRLKHHHKRW | |
| | HFTFSHFVVEPGQEYEVTVHHLPKPIPDGDPNHQSRN | |
| | FPVPGCEDPRMKMITPCVGSGSLWDPNITVETLEARQ | |
| | LWVSFTLWNESTHYQILLTSFPHTENHSCFQHTLMVP | |
| | EPAYQDSRQRSNVTLTLSDSNWCCRHRVQIQPFFSSC | |
| | LNDCLRHSITVPCPEIPDPPVSIADYIGSPKTASTIE | |
| | SKTGECPKCPVPEIPGAPSVFIFPPKPKDTLSISRTP | |
| | EVTCLVVDLGPDDSNVQITWFVDNTEMHTAKTRPREE | |
| | QFNSTYRVVSVLPILHQDWLKGKEFKCKVNSKSLPSA | |
| | MERTISKAKGQPHEPQVYVLPPTQEELSENKVSVTCL | |
| | IKGFHPPDIAVEWEITGQPEPENNYQTTPPQLDSDGT | |
| | YFLYSRLSVDRSHWQRGNTYTCSVSHEALHSHHTQKS | |
| | LTQSPGK | |
| 44 | SPRLLEHPAPVCSQQGLNCTVKNSTCLDDSWLHPPHL | Equine IL17Ra ECD-equine |
| | TPSSPKDVQIQLHFAHTQQGDLLPVIHIEWTLQTDAS | IgG-2-Fc |
| | ILYLEGAELSVLQLSTNERLCVTFEFLSRLKHHHKRW | |
| | RFTFAHFVVEPGQEYEVTVHHLPKPFPHGDPNHQSRN | |
| | FLVPDCMDPRMRITIPCVSSGSLWDPNITVETLEAHR | |
| | LRVDFTLWNESARYQILLSSFPHMENQSCFDDVQNIL | |
| | KHTPEASHQRANITLTLSDFNWCCRHHVQIQPFFSSC | |
| | LNDCLRHTVTVPCPEIPDTPDSTADYMGSDMSKCPKC | |
| | PAPELLGGPSVFIFPPNPKDALMISRIPVVTCVVVNL | |
| | SDQYPDVQFSWYVDNTEVHSAITKQREAQFNSTYRVV | |
| | SVLPIQHQDWLSGKEFKCSVTNVGVPQPISRAISRGK | |
| | GPSRVPQVYVLPPHPDELAKSKVSVTCLVKDFYPPDI | |
| | SVEWQSNRWPELEGKYSTTPAQLDGDGSYFLYSKLSL | |
| | ETSRWQQVESFTCAVMHEALHNHFTKTDISESLGK | |
| 45 | PKRENGRVPRPPDCPKCPAPEMLGGPSVFIFPPKPKD | canine IgG-B-Fc |
| | TLLIARTPEVTCVVVDLDPEDPEVQISWFVDGKQMQT | |
| | AKTQPREEQFNGTYRVVSVLPIGHQDWLKGKQFTCKV | |
| | NNKALPSPIERTISKARGQAHQPSVYVLPPSREELSK | |
| | NTVSLTCLIKDFFPPDIDVEWQSNGQQEPESKYRTTP | |
| | PQLDEDGSYFLYSKLSVDKSRWQRGDTFICAVMHEAL | |
| | HNHYTQESLSHSPGK | |
| 46 | AKECECKCNCNNCPCPGCGLLGGPSVFIFPPKPKDIL | canine IgG-C-Fc |
| | VTARTPTVTCVVVDLDPENPEVQISWFVDSKQVQTAN | |
| | TQPREEQSNGTYRVVSVLPIGHQDWLSGKQFKCKVNN | |
| | KALPSPIEEIISKTPGQAHQPNVYVLPPSRDEMSKNT | |
| | VTLTCLVKDFFPPEIDVEWQSNGQQEPESKYRMTPPQ | |
| | LDEDGSYFLYSKLSVDKSRWQRGDTFICAVMHEALHN | |
| | HYTQISLSHSPGK | |
| 47 | PKRENGRVPRPPDCPKCPAPEMLGGPSVFIFPPKPKD | canine IgG-B-Fc variant 1 |
| | TLLIARTPEVTCVVVDLDPEDPEVQISWFVDGKQMQT | (C1q binding mutant) |
| | AKTQPREEQFNGTYRVVSVLPIGHQDWLKGKQFTCRV | |
| | NNKALPSPIERTISKARGQAHQPSVYVLPPSREELSK | |
| | NTVSLTCLIKDFFPPDIDVEWQSNGQQEPESKYRTTP | |
| | PQLDEDGSYFLYSKLSVDKSRWQRGDTFICAVMHEAL | |
| | HNHYTQESLSHSPGK | |
| 48 | AKECECKCNCNNCPCPGCGLLGGPSVFIFPPKPKDIL | canine IgG-C-Fc variant 1 |
| | VTARTPTVTCVVVDLDPENPEVQISWFVDSKQVQTAN | (C1q binding mutant) |
| | TQPREEQSNGTYRVVSVLPIGHQDWLSGKQFKCRVNN | |
| | KALPSPIEEIISKTPGQAHQPNVYVLPPSRDEMSKNT | |
| | VTLTCLVKDFFPPEIDVEWQSNGQQEPESKYRMTPPQ | |
| | LDEDGSYFLYSKLSVDKSRWQRGDTFICAVMHEALHN | |
| | HYTQISLSHSPGK | |
| 49 | PKRENGRVPRPPDCPKCPAPEMLGGPSVFIFPPKPKD | canine IgG-B-Fc variant 2 |
| | TLLIARTPEVTCVVVDLGPEDPEVQISWFVDGKQMQT | (CD16 binding mutant 1) |
| | AKTQPREEQFNGTYRVVSVLPIGHQDWLKGKQFTCKV | |
| | NNIGLPSPIERTISKARGQAHQPSVYVLPPSREELSK | |
| | NTVSLTCLIKDFFPPDIDVEWQSNGQQEPESKYRTTP | |
| | PQLDEDGSYFLYSKLSVDKSRWQRGDTFICAVMHEAL | |
| | HNHYTQESLSHSPGK | |
| 50 | AKECECKCNCNNCPCPGCGLLGGPSVFIFPPKPKDIL | canine IgG-C-Fc variant 2 |
| | VTARTPTVTCVVVDLGPENPEVQISWFVDSKQVQTAN | (CD16 binding mutant 1) |
| | TQPREEQSNGTYRVVSVLPIGHQDWLSGKQFKCKVNN | |
| | IGLPSPIEEIISKTPGQAHQPNVYVLPPSRDEMSKNT | |
| | VTLTCLVKDFFPPEIDVEWQSNGQQEPESKYRMTPPQ | |
| | LDEDGSYFLYSKLSVDKSRWQRGDTFICAVMHEALHN | |
| | HYTQISLSHSPGK | |
| 51 | CRHLGCVNNEGNIN | Canine IL17A epitope C, |
| | | expanded sequence |
| 52 | GFSLTSNGVH | Variable heavy chain CDR- |
| | | H1 amino acid sequence of |
| | | mouse antibody clone A |
| 53 | WLVVIWSDGTTTYNSDFKS | Variable heavy chain CDR- |
| | | H2 amino acid sequence of |
| | | mouse antibody clone A |
| 109 | VIWSDGTTTYNSDFKS | Variable heavy chain CDR- |
| | | H2 v2 amino acid sequence |
| | | of clone A |
| 54 | ARHYDWGYYYAMDY | Variable heavy chain CDR- |
| | | H3 amino acid sequence of |
| | | mouse antibody clone A |
| 55 | QVQLKESGPGLVAPSQSLSITCTIS | Variable region heavy chain |
| | | framework HC-FR1 amino |
| | | acid sequence of mouse |
| | | antibody clone A |
| 56 | WVRQSPGKDLE | Variable region heavy chain |
| | | framework HC-FR2 amino |
| | | acid sequence of mouse |
| | | antibody clone A |
| 110 | WVRQSPGKDLEWLV | Variable region heavy chain |
| | | framework HC-FR2 v2 |
| | | amino acid sequence of |
| | | mouse antibody clone A |
| 57 | RLSISKDNSKSQVFLKMNSLQTDDTAMYYC | Variable region heavy chain |
| | | framework HC-FR3 amino |
| | | acid sequence of mouse |
| | | antibody clone A |
| 58 | WGQGTSVTVSS | Variable region heavy chain |
| | | framework HC-FR4 amino |
| | | acid sequence of mouse |
| | | antibody clone A |
| 59 | SSQSLVHSNGNTYLHWY | Variable light chain CDR-L1 |
| | | amino acid sequence of mouse |
| | | antibody clone A |
| 111 | SSQSLVHSNGNTYLH | Variable light chain CDR-L1 |
| | | v2 amino acid sequence of |
| | | mouse antibody clone A |
| 60 | LLIYKVSNRFS | Variable light chain CDR-L2 |
| | | amino acid sequence of |
| | | mouse antibody clone A |
| 112 | KVSNRFS | Variable light chain CDR-L2 |
| | | v2 amino acid sequence of |
| | | mouse antibody clone A |
| 61 | SQSTHVPFT | Variable light chain CDR-L3 |
| | | amino acid sequence of |
| | | mouse antibody clone A |
| 62 | DVVMTQTPLSLPVSLGDQASISCR | Variable region light chain |
| | | framework LC-FR1 amino |
| | | acid sequence of mouse |
| | | antibody clone A |
| 63 | LQRPGQSPN | Variable region light chain |
| | | framework LC-FR2 amino |
| | | acid sequence of mouse |
| | | antibody clone A |
| 113 | WYLQRPGQSPNLLIY | Variable region light chain |
| | | framework LC-FR2 v2 amino |
| | | acid sequence of mouse |
| | | antibody clone A |
| 64 | GVPDRFSGSGSGTDFTLKISRVEAEDLGVYFC | Variable region light chain |
| | | framework LC-FR3 amino |
| | | acid sequence of mouse |
| | | antibody clone A |
| 65 | FGSGTKLEIK | Variable region light chain |
| | | framework LC-FR4 amino |
| | | acid sequence of mouse |
| | | antibody clone A |
| 66 | GFSLTSNGVH | Variable heavy chain CDR- |
| | | H1 amino acid sequence of |
| | | mouse antibody clone E |
| 67 | WLVVIWSDGTTTYNSALKS | Variable heavy chain CDR- |
| | | H2 amino acid sequence of |
| | | mouse antibody clone E |
| 114 | VIWSDGITTYNSALKS | Variable heavy chain CDR- |
| | | H2 v2 amino acid sequence |
| | | of mouse antibody clone E |
| 68 | ARHYDRGYYYAMDY | Variable heavy chain CDR- |
| | | H3 amino acid sequence of |
| | | mouse antibody clone E |
| 69 | QVQLKESGPGLVAPSQSLSITCTIS | Variable region heavy chain |
| | | framework HC-FR1 amino |
| | | acid sequence of mouse |
| | | antibody clone E |
| 70 | WVRQPPGKGLE | Variable region heavy chain |
| | | framework HC-FR2 amino |
| | | acid sequence of mouse |
| | | antibody clone E |
| 115 | WVRQPPGKGLEWLV | Variable region heavy chain |
| | | framework HC-FR2 v2 |
| | | amino acid sequence of |
| | | mouse antibody clone E |
| 71 | RLSISKDNSKSQVFLKMNSLQTDDTAMYYC | Variable region heavy chain |
| | | framework HC-FR3 amino |
| | | acid sequence of mouse |
| | | antibody clone E |
| 72 | WGQGTSVTVSS | Variable region heavy chain |
| | | framework HC-FR4 amino |
| | | acid sequence of mouse |
| | | antibody clone E |
| 73 | RSSQSLVHSNGNTYFHWY | Variable light chain CDR- |
| | | L1 amino acid sequence of |
| | | mouse antibody clone E |
| 116 | RSSQSLVHSNGNTYFH | Variable light chain CDR- |
| | | L1 v2 amino acid sequence |
| | | of mouse antibody clone E |
| 74 | LLIYKVSNRFS | Variable light chain CDR- |
| | | L2 amino acid sequence of |
| | | mouse antibody clone E |
| 117 | KVSNRFS | Variable light chain CDR- |
| | | L2 v2 amino acid sequence |
| | | of mouse antibody clone E |
| 75 | SQSTHAPFT | Variable light chain CDR- |
| | | L3 amino acid sequence of |
| | | mouse antibody clone E |
| 76 | DVVMTQTPLSLPVSLGDQASISC | Variable region light chain |
| | | framework LC-FR1 amino |
| | | acid sequence of mouse |
| | | antibody clone E |
| 77 | LQKPGQSPK | Variable region light chain |
| | | framework LC-FR2 amino |
| | | acid sequence of mouse |
| | | antibody clone E |
| 118 | WYLQKPGQSPKLLIY | Variable region light chain |
| | | framework LC-FR2 v2 |
| | | amino acid sequence of |
| | | mouse antibody clone E |
| 78 | GVPDRFSGSGSGTDFTLKISRVEAEDLGVYFC | Variable region light chain |
| | | framework LC-FR3 amino |
| | | acid sequence of mouse |
| | | antibody clone E |
| 79 | FGSGTKLEIK | Variable region light chain |
| | | framework LC-FR4 amino |
| | | acid sequence of mouse |
| | | antibody clone E |
| 80 | GYSFTYYYMH | Variable heavy chain CDR- |
| | | H1 amino acid sequence of |
| | | mouse antibody clone D |
| 81 | WIGYISCFNGDTNYNQEFKD | Variable heavy chain CDR- |
| | | H2 amino acid sequence of |
| | | mouse antibody clone D |
| 119 | YISCFNGDTNYNQEFKD | Variable heavy chain CDR- |
| | | H2 v2 amino acid sequence |
| | | of mouse antibody clone D |
| 82 | ARGLSTLITEGWFAY | Variable heavy chain CDR- |
| | | H3 amino acid sequence of |
| | | mouse antibody clone D |
| 83 | EVQLQQSGPELVKTGASVKISCKAS | Variable region heavy chain |
| | | framework HC-FR1 amino |
| | | acid sequence of mouse |
| | | antibody clone D |
| 84 | WVKQSHGKSLE | Variable region heavy chain |
| | | framework HC-FR2 amino |
| | | acid sequence of mouse |
| | | antibody clone D |
| 120 | WVKQSHGKSLEWIG | Variable region heavy chain |
| | | framework HC-FR2 v2 |
| | | amino acid sequence of |
| | | mouse antibody clone D |
| 85 | KATFTADTSSSTAYMQFNSLTSEDSAVYYC | Variable region heavy chain |
| | | framework HC-FR3 amino |
| | | acid sequence of mouse |
| | | antibody clone D |
| 86 | WGQGTLVTVSA | Variable region heavy chain |
| | | framework HC-FR4 amino |
| | | acid sequence of mouse |
| | | antibody clone D |
| 87 | RASKSVSTSGYSYMHWN | Variable light chain CDR- |
| | | L1 amino acid sequence of |
| | | mouse antibody clone D |
| 121 | RASKSVSTSGYSYMH | Variable light chain CDR- |
| | | L1 v2 amino acid sequence |
| | | of mouse antibody clone D |
| 88 | LLIYLVSNLES | Variable light chain CDR- |
| | | L2 amino acid sequence of |
| | | mouse antibody clone D |
| 122 | LVSNLES | Variable light chain CDR- |
| | | L2 v2 amino acid sequence |
| | | of mouse antibody clone D |
| 89 | QHIRELYT | Variable light chain CDR- |
| | | L3 amino acid sequence of |
| | | mouse antibody clone D |
| 90 | DIVLTQSPASLAVSLGQRATISY | Variable region light chain |
| | | framework LC-FR1 amino |
| | | acid sequence of mouse |
| | | antibody clone D |
| 91 | QQKPGQPPR | Variable region light chain |
| | | framework LC-FR2 amino |
| | | acid sequence of mouse |
| | | antibody clone D |
| 123 | WNQQKPGQPPRLLIY | Variable region light chain |
| | | framework LC-FR2 v2 |
| | | amino acid sequence of |
| | | mouse antibody clone D |
| 92 | GVPARFSGSGSGTDFTLNIHPVEEEDAATYYC | Variable region light chain |
| | | framework LC-FR3 amino |
| | | acid sequence of mouse |
| | | antibody clone D |
| 93 | FGGGTKLEIK | Variable region light chain |
| | | framework LC-FR4 amino |
| | | acid sequence of mouse |
| | | antibody clone D |
| 94 | LGEGLNCTVKNSTCLDDSWIHPRNLTPSSPKDVQVHL | Truncated canine IL17Ra |
| | DFAQTQHGDLLPIIGIRWTLQTDASILFLEGAELSVL | ECD |
| | QLNTNERVCVKFEELSKLKHHHKRWHFTFSHFVVEPG | |
| | QEYEVTVHHLPKPIPDGDPNHQSKNFLVPGCEDPRMR | |
| | MTTPCVSSGSLWDPNITAEALEAHQLQVHFTLWNESA | |
| | QYQILLTSEPHTENRSCFHRVLMVPEPTLKEHHQRAN | |
| | IMLIGSSSNWCCRHQVQIQPFFSSCLNDCLRHSVTVP | |
| | CP | |
| 95 | MAVLGLLFCLVTFPSCVLSTETQPPVTNLSVSVENLC | IL4R/IL13R-canine IgG-B |
| | TVIWTWDPPEGASPNCTLRYFSHFDNKQDKKIAPETH | |
| | RSKEVPLNERICLQVGSQCSTNESDNPSILVEKCTPP | |
| | PEGDPESAVTELQCVWHNLSYMKCTWLPGRNTSPDTN | |
| | YTLYYWHSSLGKILQCEDIYREGQHIGCSFALTNLKD | |
| | SSFEQHSVQIVVKDNAGKIRPSFNIVPLTSHVKPDPP | |
| | HIKRLFFQNGNLYVQWKNPQNFYSRCLSYQVEVNNSQ | |
| | TETNDIFYVEEAKCQNSEFEGNLEGTICFMVPGVLPD | |
| | TLNTVRIRVRTNKLCYEDDKLWSNWSQAMSIGENTDP | |
| | TGGGSGSGSVKVLHEPSCFSDYISTSVCQWKMDHPTN | |
| | CSAELRLSYQLDFMGSENHTCVPENREDSVCVCSMPI | |
| | DDAVEADVYQLDLWAGQQLLWSGSFQPSKHVKPRTPG | |
| | NLTVHPNISHTWLLMWTNPYPTENHLHSELTYMVNVS | |
| | NDNDPEDFKVYNVTYMGPTLRLAASTLKSGASYSARV | |
| | RAWAQTYNSTWSDWSPSTTWLNYYEPKRENGRVPRPP | |
| | DCPKCPAPEMLGGPSVFIFPPKPKDTLLIARTPEVTC | |
| | VVVDLDPEDPEVQISWFVDGKQMQTAKTQPREEQFNG | |
| | TYRVVSVLPIGHQDWLKGKQFTCKVNNKALPSPIERT | |
| | ISKARGQAHQPSVYVLPPSREELSKNIVSLICLIKDF | |
| | FPPDIDVEWQSNGQQEPESKYRTTPPQLDEDGSYFLY | |
| | SKLSVDKSRWQRGDTFICAVMHEALHNHYTQESLSHS | |
| | PGK | |
| 96 | MAVLGLLFCLVTFPSCVLSTETQPPVTNLSVSVENLC | IL4R/IL13R-canine IgG-B |
| | TVIWTWDPPEGASPNCTLRYFSHFDNKQDKKIAPETH | variant 1 |
| | RSKEVPLNERICLQVGSQCSTNESDNPSILVEKCTPP | (C1q binding mutant) |
| | PEGDPESAVTELQCVWHNLSYMKCTWLPGRNTSPDTN | |
| | YTLYYWHSSLGKILQCEDIYREGQHIGCSFALTNLKD | |
| | SSFEQHSVQIVVKDNAGKIRPSFNIVPLTSHVKPDPP | |
| | HIKRLFFQNGNLYVQWKNPQNFYSRCLSYQVEVNNSQ | |
| | TETNDIFYVEEAKCQNSEFEGNLEGTICFMVPGVLPD | |
| | TLNTVRIRVRTNKLCYEDDKLWSNWSQAMSIGENTDP | |
| | TGGGSGSGSVKVLHEPSCFSDYISTSVCQWKMDHPTN | |
| | CSAELRLSYQLDFMGSENHTCVPENREDSVCVCSMPI | |
| | DDAVEADVYQLDLWAGQQLLWSGSFQPSKHVKPRTPG | |
| | NLTVHPNISHTWLLMWTNPYPTENHLHSELTYMVNVS | |
| | NDNDPEDFKVYNVTYMGPTLRLAASTLKSGASYSARV | |
| | RAWAQTYNSTWSDWSPSTTWLNYYEPKRENGRVPRPP | |
| | DCPKCPAPEMLGGPSVFIFPPKPKDTLLIARTPEVTC | |
| | VVVDLDPEDPEVQISWFVDGKQMQTAKTQPREEQFNG | |
| | TYRVVSVLPIGHQDWLKGKQFTCRVNNKALPSPIERT | |
| | ISKARGQAHQPSVYVLPPSREELSKNTVSLTCLIKDF | |
| | FPPDIDVEWQSNGQQEPESKYRTTPPQLDEDGSYFLY | |
| | SKLSVDKSRWQRGDTFICAVMHEALHNHYTQESLSHS | |
| | PGK | |
| 97 | SLRLLDHRALVCSQPGLNCTVKNSTCLDDSWIHPRNL | Human IL17Ra ECD |
| | TPSSPKDLQIQLHFAHTQQGDLFPVAHIEWTLQTDAS | |
| | ILYLEGAELSVLQLNTNERLCVRFEFLSKLRHHHRRW | |
| | RFTFSHFVVDPDQEYEVTVHHLPKPIPDGDPNHQSKN | |
| | FLVPDCEHARMKVTTPCMSSGSLWDPDITVETLEAHQ | |
| | LRVSFTLWNESTHYQILLTSFPHMENHSCFEHMHHIP | |
| | APRPEEFHQRSDVTLTLRNLKGCCRHQVQIQPFFSSC | |
| | LNDCLRHSATVSCP | |
| 98 | SPRLLDYPAPVCSQQGLNCVVKNSTCLDDSWIHLRNL | Feline IL17Ra ECD |
| | TPSSPKDVQVHLDFVQTQHGDLLPVAGIRWTLQTDAS | |
| | ILYLEGAELSVLQLNTNERLCVKFEFLTRLKHHHKRW | |
| | HFTFSHFVVEPGQEYEVTVHHLPKPIPDGDPNHQSRN | |
| | FPVPGCEDPRMKMITPCVGSGSLWDPNITVETLEARQ | |
| | LWVSFTLWNESTHYQILLTSFPHTENHSCFQHTLMVP | |
| | EPAYQDSRQRSNVTLTLSDSNWCCRHRVQIQPFFSSC | |
| | LNDCLRHSITVPCPEIPDPPVSIADYI | |
| 99 | SPRLLEHPAPVCSQQGLNCTVKNSTCLDDSWLHPPHL | Equine IL17Ra ECD |
| | TPSSPKDVQIQLHFAHTQQGDLLPVIHIEWTLQTDAS | |
| | ILYLEGAELSVLQLSTNERLCVTFEFLSRLKHHHKRW | |
| | RFTFAHFVVEPGQEYEVTVHHLPKPFPHGDPNHQSRN | |
| | FLVPDCMDPRMRITTPCVSSGSLWDPNITVETLEAHR | |
| | LRVDFTLWNESARYQILLSSFPHMENQSCFDDVQNIL | |
| | KHTPEASHQRANITLTLSDFNWCCRHHVQIQPFFSSC | |
| | LNDCLRHTVTVPCPEIPDTPDSTADYM | |
| 100 | MKFPSQLLLFLLFRITGIICDIQMTQSSSYLSVSLGG | Mouse monoclonal antibody |
| | RVTITCKANDHINNWLAWYQQKPGNAPRLLISGSTSL | Clone C variable light chain, |
| | ETGVPSRFSGSGSGKDYTLSITSLQTEDVATYYCQQY | with leader sequence and |
| | WSTPFTFGSGTKLEIKRADAAPTVSIFPPSSEQLTSG | certain C-terminal sequence |
| | GASVVLGVIMVIAVSCVKLLSAHNST | |
| 101 | MKLPVRLLVLMFWIPASNSDVVMTQTPLSLPVSLGDQ | Mouse monoclonal antibody |
| | ASISCRSSQSLVHSNGNTYFHWYLQKPGQSPKLLIYK | Clone E variable light chain, |
| | VSNRFSGVPDRFSGSGSGTDFTLKISRVEAEDLGVYF | with leader sequence and |
| | CSQSTHAPFTFGSGTKLEIKRADAAPTVSIFPPSSEQ | certain C-terminal sequence |
| | LTSGGASVVSRAN | |
| 102 | MKLPVRLLVLMFWIPASSSDVVMTQTPLSLPVSLGDQ | Mouse monoclonal antibody |
| | ASISCRSSQSLVHSNGNTYLHWYLQRPGQSPNLLIYK | Clone A variable light |
| | VSNRFSGVPDRFSGSGSGTDFTLKISRVEAEDLGVYF | chain, with leader sequence |
| | CSQSTHVPFTFGSGTKLEIKRADAAPTVSIFPPSSEQ | and certain C-terminal |
| | LTSGGASVVCKGEF | sequence |
| 103 | METDTLLLWVLLLWVPGSTGDIVLTQSPASLAVSLGQ | Mouse monoclonal antibody |
| | RATISYRASKSVSTSGYSYMHWNQQKPGQPPRLLIYL | Clone D variable light |
| | VSNLESGVPARFSGSGSGTDFTLNIHPVEEEDAATYY | chain, with leader sequence |
| | CQHIRELYTFGGGTKLEIKRADAAPTVSI | and certain C-terminal |
| | | sequence |
| 104 | MGWIWIFLFLLSGTAGVHSEVQLQQSGPELVKTGASV | Mouse monoclonal antibody |
| | KISCKASGYSFTYYYMHWVKQSHGKSLEWIGYISCFN | Clone D variable heavy |
| | GDTNYNQEFKDKATFTADTSSSTAYMQFNSLTSEDSA | chain, with leader sequence |
| | VYYCARGLSTLITEGWFAYWGQGTLVTVSAAKTTPPS | and certain C-terminal |
| | VYPLAPGSA | sequence |
| 105 | MNLGLSFIFLALILKGVQCEVQLVESGGGLVQPGGSL | Mouse monoclonal antibody |
| | KLSCAASGFTFSSYGMSWVRQTPDKRLELVAIINSNG | Clone C variable heavy |
| | GSTYYPDSVKGRFTISRDNDKNSLYLQMSSLKSEDTA | chain, with leader sequence |
| | MYYCVRCHYDYERVFDYWGQGTTLTVSSAKTTPPSVY | and certain C-terminal |
| | PLAPGSAAQTNSMVTLGCLVKGYFPE | sequence |
| 106 | MAVLGLLLCLVTFPSCVLSQVQLKESGPGLVAPSQSL | Mouse monoclonal antibody |
| | SITCTISGFSLTSNGVHWVRQSPGKDLEWLVVIWSDG | Clone A variable heavy |
| | TTTYNSDFKSRLSISKDNSKSQVFLKMNSLQTDDTAM | chain, with leader sequence |
| | YYCARHYDWGYYYAMDYWGQGTSVTVSSAKTTPPSVY | and certain C-terminal |
| | PLAPGSAAQTNSMVTLGCLVKGEF | sequence |
| 107 | MAVLGLLLCLVTFPSCVLSQVQLKESGPGLVAPSQSL | Mouse monoclonal antibody |
| | SITCTISGFSLTSNGVHWVRQPPGKGLEWLVVIWSDG | Clone E variable heavy |
| | TTTYNSALKSRLSISKDNSKSQVFLKMNSLQTDDTAM | chain, with leader sequence |
| | YYCARHYDRGYYYAMDYWGQGTSVTVSSAKTTPPSVY | and certain C-terminal |
| | PLAPGSAAQTNSMVTLGCLVKGEF | sequence |

### DESCRIPTION OF CERTAIN EMBODIMENTS

Antibodies that bind canine IL17A, feline IL17A, or equine IL17A are provided. Antibody heavy chains and light chains that are capable of forming antibodies that bind IL17A are also provided. In addition, antibodies, heavy chains, and light chains comprising one or more particular complementary determining regions (CDRs) are provided. The present disclosure also provides polypeptides comprising an IL17Ra ECD polypeptide that are capable of binding IL17A. Polynucleotides encoding antibodies to IL17A and polypeptides comprising an IL17Ra ECD polypeptide are provided as well as methods of producing and purifying the antibodies and polypeptides. Methods of treatment using antibodies to IL17A and polypeptides comprising an IL17A ECD polypeptide to bind IL17A and inhibit IL17A-mediated signaling are provided. Such methods include, but are not limited to, methods of treating IL17A-induced conditions in a subject, such as companion animal species. Methods of detecting IL17A in a sample from a companion animal species are also provided.

The present disclosure also provides IgG Fc variant polypeptides having one or more amino acid substitutions and reducing binding to C1 q and/or CD16 and methods of producing and using the same. For example, IgG Fc variants and/or polypeptides comprising the IgG Fc variants (e.g., fusion polypeptides comprising the IgG Fc variants and the anti-IL17A antibodies and/or IL17Ra ECD polypeptides described herein) may have reduced complement-mediated immune responses and/or antibody-dependent cell-mediated cytotoxicity.

For the convenience of the reader, the following definitions of terms used herein are provided.

As used herein, numerical terms such as Kd are calculated based upon scientific measurements and, thus, are subject to appropriate measurement error. In some instances, a numerical term may include numerical values that are rounded to the nearest significant figure.

As used herein, “a” or “an” means “at least one” or “one or more” unless otherwise specified. As used herein, the term “or” means “and/or” unless specified otherwise. In the context of a multiple dependent claim, the use of “or” when referring back to other claims refers to those claims in the alternative only.

### Exemplary Anti-IL17 Antibodies

Novel antibodies directed against IL17A are provided, for example antibodies that bind to canine IL17A, feline IL17A, and/or equine IL17A. Anti-IL17 antibodies provided herein include, but are not limited to, monoclonal antibodies, mouse antibodies, chimeric antibodies, caninized antibodies, felinized antibodies, and equinized antibodies. In some embodiments, an anti-IL17A antibody is an isolated mouse monoclonal antibody such as Clone C, Clone A, Clone D, and Clone E.

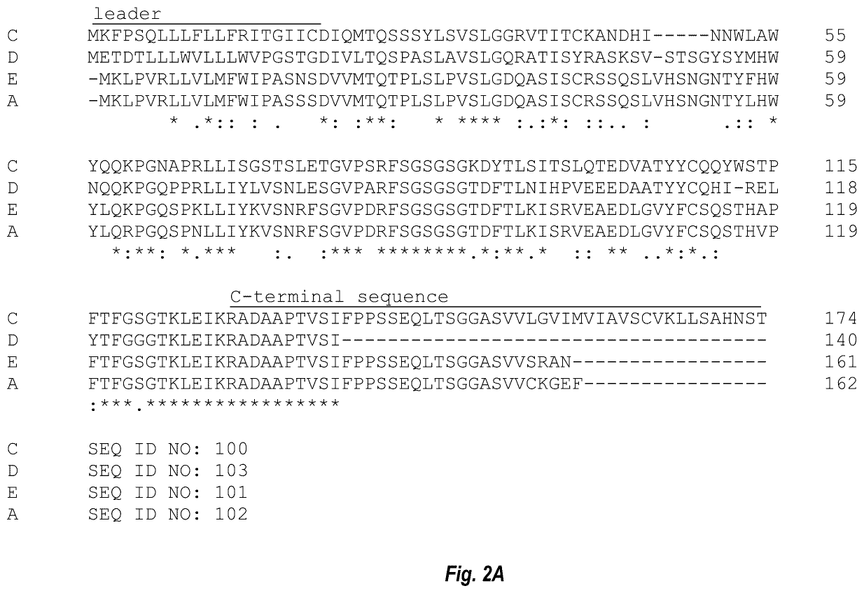

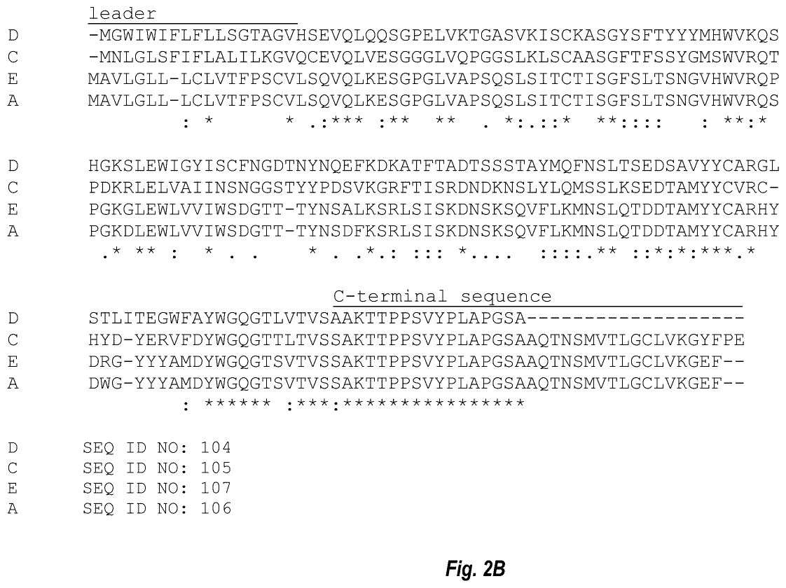

A hybridoma bank generated from immunization of mice with human IL17A was screened for affinity to canine IL17A by enzyme linked immunosorbent assay (ELISA). Monoclonal antibodies Clone C, Clone A, Clone D, and Clone E were selected for further investigation. The variable heavy chain (VH) and variable light chain (VL) of each of the four clones were sequenced and analyzed by sequence alignment (FIG. 2).

Provided herein are amino acid sequences of monoclonal antibody Clone C. For example, the variable heavy chain CDRs (SEQ ID NOs: 1-3), variable light chain CDRs (SEQ ID NOs: 8, 9 or 108, and 10), variable region heavy chain framework sequences (SEQ ID NOs: 4-7), and variable region light chain framework sequences (SEQ ID NOs: 11-14) for monoclonal antibody Clone C are provided. The amino acid sequences of the variable light chain and variable heavy chain of monoclonal antibody Clone C are provided (SEQ ID NOs: 24 and 25, respectively).

Also provided herein are amino acid sequences of monoclonal antibody Clone A. For example, the variable heavy chain CDRs (SEQ ID NOs: 52, 53 or 109, and 54), variable light chain CDRs (SEQ ID NOs: 59 or 111, 60 or 112, and 61), variable region heavy chain framework sequences (SEQ ID NOs: 55, 56 or 110, 57, and 58), and variable region light chain framework sequences (SEQ ID NOs: 62, 63 or 113, 64, and 65) for monoclonal antibody Clone A are provided. The amino acid sequences of the variable light chain and variable heavy chain of monoclonal antibody Clone A are provided (SEQ ID NOs: 34 and 35, respectively).

In addition, provided herein are amino acid sequences of monoclonal antibody Clone D. For example, the variable heavy chain CDRs (SEQ ID NOs: 80, 81 or 119, and 82), variable light chain CDRs (SEQ ID NOs: 87 or 121, 88 or 122, and 89), variable region heavy chain framework sequences (SEQ ID NOs: 83, 84 or 120, 85, and 86), and variable region light chain framework sequences (SEQ ID NOs: 90, 91 or 123, 92, and 93) for monoclonal antibody Clone D are provided. The amino acid sequences of the variable light chain and variable heavy chain of monoclonal antibody Clone D are provided (SEQ ID NOs: 36 and 37, respectively).

Provided herein are amino acid sequences of monoclonal antibody Clone E. For example, the variable heavy chain CDRs (SEQ ID NOs: 66, 67 or 114, and 68), variable light chain CDRs (SEQ ID NOs: 73 or 116, 74 or 117, and 75), variable region heavy chain framework sequences (SEQ ID NOs: 69, 70 or 115, 71, and 72), and variable region light chain framework sequences (SEQ ID NOs: 76, 77 or 118, 78, and 79) for monoclonal antibody Clone A are provided. The amino acid sequences of the variable light chain and variable heavy chain of monoclonal antibody Clone A are provided (SEQ ID NOs: 38 and 39, respectively).

Also provided herein are chimeric, caninized, felinized, and equinized antibodies derived from Clone C, A, D, and E antibodies. For example, in some embodiments, amino acid sequences of caninized monoclonal antibody Clone C are provided, such as SEQ ID NOs: 15-21. In some embodiments, amino acid sequences of chimeric antibodies derived from monoclonal antibody Clone C are provided, such as SEQ ID NOs: 26 and 27.

The term “antibody” herein is used in the broadest sense and encompasses various antibody structures, including but not limited to monoclonal antibodies, polyclonal antibodies, multispecific antibodies (for example, bispecific (such as Bi-specific T-cell engagers) and trispecific antibodies), and antibody fragments (such as Fab, F(ab′)2, ScFv, minibody, diabody, triabody, and tetrabody) so long as they exhibit the desired antigen-binding activity. Canine, feline, and equine species have different varieties (classes) of antibodies that are shared by many mammalians.

The term antibody includes, but is not limited to, fragments that are capable of binding to an antigen, such as Fv, single-chain Fv (scFv), Fab, Fab′, di-scFv, sdAb (single domain antibody) and (Fab′)2 (including a chemically linked F(ab′)2). Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen combining sites and is still capable of cross-linking antigen. The term antibody also includes, but is not limited to, chimeric antibodies, humanized antibodies, and antibodies of various species such as mouse, human, cynomolgus monkey, canine, feline, equine, etc. Furthermore, for all antibody constructs provided herein, variants having the sequences from other organisms are also contemplated. Thus, if a murine version of an antibody is disclosed, one of skill in the art will appreciate how to transform the murine sequence based antibody into a cat, dog, horse, etc. sequence. Antibody fragments also include either orientation of single chain scFvs, tandem di-scFv, diabodies, tandem tri-sdcFv, minibodies, etc. Antibody fragments also include nanobodies (sdAb, an antibody having a single, monomeric domain, such as a pair of variable domains of heavy chains, without a light chain). An antibody fragment can be referred to as being a specific species in some embodiments (for example, mouse scFv or a canine scFv). This denotes the sequences of at least part of the non-CDR regions, rather than the source of the construct. In some embodiments, the antibodies comprise a label or are conjugated to a second moiety.

The terms “label” and “detectable label” mean a moiety attached to an antibody or its analyte to render a reaction (for example, binding) between the members of the specific binding pair, detectable. The labeled member of the specific binding pair is referred to as “detectably labeled.” Thus, the term “labeled binding protein” refers to a protein with a label incorporated that provides for the identification of the binding protein. In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels for polypeptides include, but are not limited to, the following: radioisotopes or radionuclides (for example, 3H, 14C, 35S, 90Y, 99Tc, 111In, 125I, 131I, 177Lu, 166Ho, or 153Sm); chromogens, fluorescent labels (for example, FITC, rhodamine, lanthanide phosphors), enzymatic labels (for example, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In this regard, the moiety itself may not be detectably labeled but may become detectable upon reaction with yet another moiety.

The term “monoclonal antibody” refers to an antibody of a substantially homogeneous population of antibodies, that is, the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. Thus, a sample of monoclonal antibodies can bind to the same epitope on the antigen. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

In some embodiments, the monoclonal antibody is an isolated mouse antibody selected from Clone C, Clone A, Clone D, and Clone E.

“Amino acid sequence,” means a sequence of amino acids residues in a peptide or protein. The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

“IL17A” as used herein refers to any native IL17A that results from expression and processing of IL17A in a cell. The term includes IL17A from any vertebrate source, including mammals such as primates (e.g., humans and cynomolgus monkeys) and rodents (e.g., mice and rats), and companion animals (e.g., dogs, cats, and equine), unless otherwise indicated. The term also includes naturally occurring variants of IL17A, e.g., splice variants or allelic variants.

The term “companion animal species” refers to an animal suitable to be a companion to humans. In some embodiments, a companion animal species is a small mammal, such as a canine, feline, dog, cat, horse, rabbit, ferret, guinea pig, rodent, etc. In some embodiments, a companion animal species is a farm animal, such as a horse, cow, pig, etc.

In some embodiments, a canine IL17A comprises the amino acid sequence of SEQ ID NO: 22 and SEQ ID NO: 28. In some embodiments, a feline IL17A comprises the amino acid sequence of SEQ ID NO: 30. In some embodiments, an equine IL17A comprises the amino acid sequence of SEQ ID NO: 29.

The term “IL17A binding domain” of an antibody means the binding domain formed by a light chain and heavy chain of an anti-IL17A antibody, which binds IL17A.

In some embodiments, the IL17A binding domain binds IL17A of one or more species. In some embodiments, the IL17A binding domain binds IL17A from one or more companion animal species, such as canine IL17A, feline IL17A or equine IL17A.

As used herein, the term “epitope” refers to a site on a target molecule (for example, an antigen, such as a protein, nucleic acid, carbohydrate or lipid) to which an antigen-binding molecule (for example, an antibody, antibody fragment, or scaffold protein containing antibody binding regions) binds. Epitopes often include a chemically active surface grouping of molecules such as amino acids, polypeptides or sugar side chains and have specific three-dimensional structural characteristics as well as specific charge characteristics. Epitopes can be formed both from contiguous or juxtaposed noncontiguous residues (for example, amino acids, nucleotides, sugars, lipid moiety) of the target molecule. Epitopes formed from contiguous residues (for example, amino acids, nucleotides, sugars, lipid moiety) typically are retained on exposure to denaturing solvents whereas epitopes formed by tertiary folding typically are lost on treatment with denaturing solvents. An epitope may include but is not limited to at least 3, at least 5 or 8-10 residues (for example, amino acids or nucleotides). In some examples an epitope is less than 20 residues (for example, amino acids or nucleotides) in length, less than 15 residues or less than 12 residues. Two antibodies may bind the same epitope within an antigen if they exhibit competitive binding for the antigen. In some embodiments, an epitope can be identified by a certain minimal distance to a CDR residue on the antigen-binding molecule. In some embodiments, an epitope can be identified by the above distance, and further limited to those residues involved in a bond (for example, a hydrogen bond) between an antibody residue and an antigen residue. An epitope can be identified by various scans as well, for example an alanine or arginine scan can indicate one or more residues that the antigen-binding molecule can interact with. Unless explicitly denoted, a set of residues as an epitope does not exclude other residues from being part of the epitope for a particular antibody. Rather, the presence of such a set designates a minimal series (or set of species) of epitopes. Thus, in some embodiments, a set of residues identified as an epitope designates a minimal epitope of relevance for the antigen, rather than an exclusive list of residues for an epitope on an antigen.

In some embodiments, the antibody binds to an epitope within amino acids 65 to 88 of SEQ ID NO: 22. In some embodiments, the epitope comprises the amino acid sequence of SEQ ID NO: 23 or SEQ ID NO: 51.

The term “CDR” means a complementarity determining region as defined by at least one manner of identification to one of skill in the art. In some embodiments, CDRs can be defined in accordance with any of the Chothia numbering schemes, the Kabat numbering scheme, a combination of Kabat and Chothia, the AbM definition, the contact definition, or a combination of the Kabat, Chothia, AbM, or contact definitions. The various CDRs within an antibody can be designated by their appropriate number and chain type, including, without limitation as CDR-H1, CDR H2, CDR-H3, CDR-L1, CDR-L2, and CDR-L3. The term “CDR” is used herein to also encompass a “hypervariable region” or HVR, including hypervariable loops.

In some embodiments, an anti-IL17A antibody comprises a heavy chain comprising (a) a CDR-H1 comprising the amino acid sequence of SEQ ID NO: 1; (b) a CDR-H2 comprising the amino acid sequence of SEQ ID NO: 2; or (c) a CDR-H3 comprising the amino acid sequence of SEQ ID NO: 3. In some embodiments, an anti-IL17A antibody comprises a light chain comprising (a) a CDR-L1 comprising the amino acid sequence of SEQ ID NO: 8; (b) a CDR-L2 comprising the amino acid sequence of SEQ ID NO: 9 or SEQ ID NO: 108; or (c) a CDR-L3 comprising the amino acid sequence of SEQ ID NO: 10.

In some embodiments, an anti-IL17A antibody comprises a heavy chain comprising (a) a CDR-H1 comprising the amino acid sequence of SEQ ID NO: 52, (b) a CDR-H2 comprising the amino acid sequence of SEQ ID NO: 53 or SEQ ID NO: 109, and (c) a CDR-H3 comprising the amino acid sequence of SEQ ID NO: 54; and a light chain comprising (a) a CDR-L1 comprising the amino acid sequence of SEQ ID NO: 59 or SEQ ID NO: 111, (b) a CDR-L2 comprising the amino acid sequence of SEQ ID NO: 60 or SEQ ID NO: 112, and (c) a CDR-L3 comprising the amino acid sequence of SEQ ID NO: 61.

In some embodiments, an anti-IL17A antibody comprises a heavy chain comprising (a) a CDR-H1 comprising the amino acid sequence of SEQ ID NO: 66, (b) a CDR-H2 comprising the amino acid sequence of SEQ ID NO: 67 or SEQ ID NO: 114, and (c) a CDR-H3 comprising the amino acid sequence of SEQ ID NO: 68; and a light chain comprising (a) a CDR-L1 comprising the amino acid sequence of SEQ ID NO: 73 or SEQ ID NO: 116, (b) a CDR-L2 comprising the amino acid sequence of SEQ ID NO: 74 or SEQ ID NO: 117, and (c) a CDR-L3 comprising the amino acid sequence of SEQ ID NO: 75.

In some embodiments, an anti-IL17A antibody comprises a heavy chain comprising (a) a CDR-H1 comprising the amino acid sequence of SEQ ID NO: 80, (b) a CDR-H2 comprising the amino acid sequence of SEQ ID NO: 81 or SEQ ID NO: 119, and (c) a CDR-H3 comprising the amino acid sequence of SEQ ID NO: 82; and a light chain comprising (a) a CDR-L1 comprising the amino acid sequence of SEQ ID NO: 87 or SEQ ID NO: 121, (b) a CDR-L2 comprising the amino acid sequence of SEQ ID NO: 88 or SEQ ID NO: 122, and (c) a CDR-L3 comprising the amino acid sequence of SEQ ID NO: 89.

The term “variable region” as used herein refers to a region comprising at least three CDRs. In some embodiments, the variable region includes the three CDRs and at least one framework region (“FR”). The terms “heavy chain variable region” or “variable heavy chain” are used interchangeably to refer to a region comprising at least three heavy chain CDRs. The terms “light chain variable region” or “variable light chain” are used interchangeably to refer to a region comprising at least three light chain CDRs.

In some embodiments, the variable heavy chain or variable light chain comprises at least one framework region. In some embodiments, an antibody comprises at least one heavy chain framework region selected from HC-FR1, HC-FR2, HC-FR3, and HC-FR4. In some embodiments, an antibody comprises at least one light chain framework region selected from LC-FR1, LC-FR2, LC-FR3, and LC-FR4. The framework regions may be juxtaposed between light chain CDRs or between heavy chain CDRs. For example, an antibody may comprise a variable heavy chain having the following structure: (HC-FR1)-(CDR-H1)-(HC-FR2)-(CDR-H2)-(HC-FR3)-(CDR-H3)-(HC-FR4). An antibody may comprise a variable heavy chain having the following structure: (CDR-H1)-(HC-FR2)-(CDR-H2)-(HC-FR3)-(CDR-H3). An antibody may also comprise a variable light chain having the following structure: (LC-FR1)-(CDR-L1)-(LC-FR2)-(CDR-L2)-(LC-FR3)-(CDR-L3)-(LC-FR4). An antibody may also comprise a variable light chain having the following structure: (CDR-L1)-(LC-FR2)-(CDR-L2)-(LC-FR3)-(CDR-L3).

In some embodiments, an anti-IL17A antibody comprises one or more of (a) a variable region heavy chain framework 1 (HC FR1) sequence of SEQ ID NO: 4, (b) a HC-FR2 sequence of SEQ ID NO: 5, (c) a HC-FR3 sequence of SEQ ID NO: 6, (d) a HC-FR4 sequence of SEQ ID NO: 7, (e) a variable region light chain framework 1 (LC-FR1) sequence of SEQ ID NO: 11, (f) an LC FR2 sequence of SEQ ID NO: 12, (g) an LC FR3 sequence of SEQ ID NO: 13, or (h) an LC-FR4 sequence of SEQ ID NO: 14.

In some embodiments, an anti-IL17A antibody comprises a variable light chain sequence of (a) SEQ ID NO: 16 or (b) SEQ ID NO: 24. In some embodiments, an anti-IL17A antibody comprises a variable heavy chain sequence of (a) SEQ ID NO: 15 or (b) SEQ ID NO: 25. In some embodiments, an anti-IL17A antibody comprises (a) a variable light chain sequence of SEQ ID NO: 16 and a variable heavy chain sequence of SEQ ID NO: 15 or (b) a variable light chain sequence of SEQ ID NO: 24 and a variable heavy chain sequence of SEQ ID NO: 25.

In some embodiments, an anti-IL17A antibody comprises one or more of (a) a variable region heavy chain framework 1 (HC FR1) sequence of SEQ ID NO: 55, (b) a HC-FR2 sequence of SEQ ID NO: 56 or SEQ ID NO: 110, (c) a HC-FR3 sequence of SEQ ID NO: 57, (d) a HC-FR4 sequence of SEQ ID NO: 58, (e) a variable region light chain framework 1 (LC-FR1) sequence of SEQ ID NO: 62, (f) an LC FR2 sequence of SEQ ID NO: 63 or SEQ ID NO: 113, (g) an LC FR3 sequence of SEQ ID NO: 64, or (h) an LC-FR4 sequence of SEQ ID NO: 65.

In some embodiments, an anti-IL17A antibody comprises a variable light chain sequence of SEQ ID NO: 34. In some embodiments, an anti-IL17A antibody comprises a variable heavy chain sequence of SEQ ID NO: 35. In some embodiments, an anti-IL17A antibody comprises a variable light chain sequence of SEQ ID NO: 34 and a variable heavy chain sequence of SEQ ID NO: 35.

In some embodiments, an anti-IL17A antibody comprises one or more of (a) a variable region heavy chain framework 1 (HC FR1) sequence of SEQ ID NO: 69, (b) a HC-FR2 sequence of SEQ ID NO: 70 or SEQ ID NO: 115, (c) a HC-FR3 sequence of SEQ ID NO: 71, (d) a HC-FR4 sequence of SEQ ID NO: 72, (e) a variable region light chain framework 1 (LC-FR1) sequence of SEQ ID NO: 76, (f) an LC FR2 sequence of SEQ ID NO: 77 or SEQ ID NO: 118, (g) an LC FR3 sequence of SEQ ID NO: 78, or (h) an LC-FR4 sequence of SEQ ID NO: 79.

In some embodiments, an anti-IL17A antibody comprises a variable light chain sequence of SEQ ID NO: 38. In some embodiments, an anti-IL17A antibody comprises a variable heavy chain sequence of SEQ ID NO: 39. In some embodiments, an anti-IL17A antibody comprises a variable light chain sequence of SEQ ID NO: 38 and a variable heavy chain sequence of SEQ ID NO: 39.

In some embodiments, an anti-IL17A antibody comprises one or more of (a) a variable region heavy chain framework 1 (HC FR1) sequence of SEQ ID NO: 83, (b) a HC-FR2 sequence of SEQ ID NO: 84 or SEQ ID NO: 120, (c) a HC-FR3 sequence of SEQ ID NO: 85, (d) a HC-FR4 sequence of SEQ ID NO: 86, (e) a variable region light chain framework 1 (LC-FR1) sequence of SEQ ID NO: 90, (f) an LC FR2 sequence of SEQ ID NO: 91 or SEQ ID NO: 123, (g) an LC FR3 sequence of SEQ ID NO: 92, or (h) an LC-FR4 sequence of SEQ ID NO: 93.

In some embodiments, an anti-IL17A antibody comprises a variable light chain sequence of SEQ ID NO: 36. In some embodiments, an anti-IL17A antibody comprises a variable heavy chain sequence of SEQ ID NO: 37. In some embodiments, an anti-IL17A antibody comprises a variable light chain sequence of SEQ ID NO: 36 and a variable heavy chain sequence of SEQ ID NO: 37.

The term “constant region” as used herein refers to a region comprising at least three constant domains. The terms “heavy chain constant region” or “constant heavy chain” are used interchangeably to refer to a region comprising at least three heavy chain constant domains, CH1, CH2, and CH3. Nonlimiting exemplary heavy chain constant regions include γ, δ, α, ε, and μ. Each heavy chain constant region corresponds to an antibody isotype. For example, an antibody comprising a γ constant region is an IgG antibody, an antibody comprising a δ constant region is an IgD antibody, an antibody comprising an α constant region is an IgA antibody, an antibody comprising a μ constant region is an IgM antibody, and an antibody comprising an E constant region is an IgE antibody. Certain isotypes can be further subdivided into subclasses. For example, IgG antibodies include, but are not limited to, IgG1 (comprising a γ1 constant region), IgG2 (comprising a γ2 constant region), IgG3 (comprising a γ3 constant region), and IgG4 (comprising a γ4 constant region) antibodies; IgA antibodies include, but are not limited to, IgA1 (comprising an α1 constant region) and IgA2 (comprising an α2 constant region) antibodies; and IgM antibodies include, but are not limited to IgM1 and IgM2. The terms “light chain constant region” or “constant light chain” are used interchangeably to refer to a region comprising a light chain constant domain, CL. Nonlimiting exemplary light chain constant regions include A and K. Non-function-altering deletions and alterations within the domains are encompassed within the scope of the term “constant region” unless designated otherwise. Canine, feline, and equine have antibody classes such as IgG, IgA, IgD, IgE, and IgM. Within the canine IgG antibody class are IgG-A, IgG-B, IgG C, and IgG-D. Within the feline IgG antibody class are IgG1a, IgG1 b, and IgG2. Within the equine IgG antibody class are IgG1, IgG2, IgG3, IgG4, IgG5, IgG6, and IgG7.

The term “chimeric antibody” or “chimeric” refers to an antibody in which a portion of the heavy chain or light chain is derived from a particular source or species, while at least a part of the remainder of the heavy chain or light chain is derived from a different source or species. In some embodiments, a chimeric antibody refers to an antibody comprising at least one variable region from a first species (such as mouse, rat, cynomolgus monkey, etc.) and at least one constant region from a second species (such as human, dog, cat, equine, etc.). In some embodiments, a chimeric antibody comprises at least one mouse variable region and at least one canine constant region. In some embodiments, a chimeric antibody comprises at least one mouse variable region and at least one feline constant region. In some embodiments, all of the variable regions of a chimeric antibody are from a first species and all of the constant regions of the chimeric antibody are from a second species. In some embodiments, a chimeric antibody comprises a constant heavy chain region or constant light chain region from a companion animal. In some embodiments, a chimeric antibody comprises a mouse variable heavy and light chains and a companion animal constant heavy and light chains. For example, a chimeric antibody may comprise a mouse variable heavy and light chains and a canine constant heavy and light chains; a chimeric antibody may comprise a mouse variable heavy and light chains and a feline constant heavy and light chains; or a chimeric antibody may comprise a mouse variable heavy and light chains and an equine constant heavy and light chains.

In some embodiments, an anti-IL17A antibody comprises a light chain sequence of SEQ ID NO: 26. In some embodiments, an anti-IL17A antibody comprises a heavy chain sequence of SEQ ID NO: 27. In some embodiments, an anti-IL17A antibody comprises a light chain sequence of SEQ ID NO: 26 and a variable heavy chain sequence of SEQ ID NO: 27.

A“canine chimeric” or “canine chimeric antibody” refers to a chimeric antibody having at least a portion of a heavy chain or a portion of a light chain derived from a dog. A “feline chimeric” or “feline chimeric antibody” refers to a chimeric antibody having at least a portion of a heavy chain or a portion of a light chain derived from a cat. An “equine chimeric” or “equine chimeric antibody” refers to a chimeric antibody having at least a portion of a heavy chain or a portion of a light chain derived from a horse. In some embodiments, a canine chimeric antibody comprises a mouse variable heavy and light chains and a canine constant heavy and light chains. In some embodiments, a feline chimeric antibody comprises a mouse variable heavy and light chains and a feline constant heavy and light chains. In some embodiments, an equine chimeric antibody comprises a mouse variable heavy and light chains and an equine constant heavy and light chains. In some embodiments, the antibody is a chimeric antibody comprising murine variable heavy chain framework regions or murine variable light chain framework regions.

A“canine antibody” as used herein encompasses antibodies produced in a canine; antibodies produced in non-canine animals that comprise canine immunoglobulin genes or comprise canine immunoglobulin peptides; or antibodies selected using in vitro methods, such as phage display, wherein the antibody repertoire is based on a canine immunoglobulin sequence. The term “canine antibody” denotes the genus of sequences that are canine sequences. Thus, the term is not designating the process by which the antibody was created, but the genus of sequences that are relevant.

In some embodiments, an anti-IL17A antibody comprises a canine heavy chain constant region selected from an IgG-A, IgG-B, IgG-C, and IgG-D constant region. In some embodiments, an anti-IL17A antibody is a canine IgG-A, IgG B, IgG-C, or IgG-D antibody. In some embodiments, an anti-IL17A antibody comprises (a) a heavy chain amino acid sequence of SEQ ID NO: 17; (b) a heavy chain amino acid sequence of SEQ ID NO: 18; (c) a heavy chain amino acid sequence of SEQ ID NO: 19; or (d) a heavy chain amino acid sequence of SEQ ID NO: 20.

A“feline antibody” as used herein encompasses antibodies produced in a feline; antibodies produced in non-feline animals that comprise feline immunoglobulin genes or comprise feline immunoglobulin peptides; or antibodies selected using in vitro methods, such as phage display, wherein the antibody repertoire is based on a feline immunoglobulin sequence. The term “feline antibody” denotes the genus of sequences that are feline sequences. Thus, the term is not designating the process by which the antibody was created, but the genus of sequences that are relevant.

In some embodiments, an anti-IL17A antibody comprises a feline heavy chain constant region selected from an IgG1a, IgG1 b, and IgG2 constant region. In some embodiments, an anti-IL17A antibody is a feline IgG1a, IgG1 b, or IgG2 antibody.

An “equine antibody” as used herein encompasses antibodies produced in an equine; antibodies produced in non-equine animals that comprise equine immunoglobulin genes or comprise equine immunoglobulin peptides; or antibodies selected using in vitro methods, such as phage display, wherein the antibody repertoire is based on an equine immunoglobulin sequence. The term “equine antibody” denotes the genus of sequences that are equine sequences. Thus, the term is not designating the process by which the antibody was created, but the genus of sequences that are relevant.

In some embodiments, an anti-IL17A antibody comprises an equine heavy chain constant region selected from an IgG1, IgG2, IgG3, IgG4, IgG5, IgG6 and IgG7 constant region. In some embodiments, an anti-IL17A antibody is an equine IgG1, IgG2, IgG3, IgG4, IgG5, IgG6 and IgG7 antibody.

A“caninized antibody” means an antibody in which at least one amino acid in a portion of a non-canine variable region has been replaced with the corresponding amino acid from a canine variable region. In some embodiments, a caninized antibody comprises at least one canine constant region (e.g., a γ constant region, an α constant region, a δ constant region, an ε constant region, a μ constant region, or etc.) or fragment thereof. In some embodiments, a caninized antibody is an antibody fragment, such as Fab, scFv, (Fab′)2, etc. The term “caninized” also denotes forms of non-canine (for example, murine) antibodies that are chimeric immunoglobulins, immunoglobulin chains, or fragments thereof (such as Fv, Fab, Fab′, F(ab′)2 or other antigen-binding sequences of antibodies) that contain minimal sequence of non-canine immunoglobulin. Caninized antibodies can include canine immunoglobulins (recipient antibody) in which residues from a CDR of the recipient are substituted by residues from a CDR of a non-canine species (donor antibody) such as mouse, rat, or rabbit having the desired specificity, affinity, and capacity. In some instances, Fv framework region (FR) residues of the canine immunoglobulin are replaced by corresponding non-canine residues. Furthermore, the caninized antibody can comprise residues that are found neither in the recipient antibody nor in the imported CDR or framework sequences, but are included to further refine and optimize antibody performance.

In some embodiments, at least one amino acid residue in a portion of a mouse variable heavy chain or a mouse variable light chain has been replaced with the corresponding amino acid from a canine variable region. In some embodiments, the modified chain is fused to a canine constant heavy chain or a canine constant light chain. In some embodiments, an anti-IL17A antibody comprises (a) a heavy chain sequence of SEQ ID NO: 15; (b) a heavy chain sequence of SEQ ID NO: 17; (c) a heavy chain sequence of SEQ ID NO: 18; (d) a heavy chain sequence of SEQ ID NO: 19; (e) a heavy chain sequence of SEQ ID NO: 20; or (f) a light chain sequence of SEQ ID NO: 16; or (g) a light chain sequence of SEQ ID NO: 21.

A“felinized antibody” means an antibody in which at least one amino acid in a portion of a non-feline variable region has been replaced with the corresponding amino acid from a feline variable region. In some embodiments, a felinized antibody comprises at least one feline constant region (e.g., a γ constant region, an α constant region, a δ constant region, an ε constant region, a μ constant region, or etc.) or fragment thereof. In some embodiments, a felinized antibody is an antibody fragment, such as Fab, scFv, (Fab′)2, etc. The term “felinized” also denotes forms of non-feline (for example, murine) antibodies that are chimeric immunoglobulins, immunoglobulin chains, or fragments thereof (such as Fv, Fab, Fab′, F(ab′)2 or other antigen-binding sequences of antibodies) that contain minimal sequence of non-feline immunoglobulin. Felinized antibodies can include feline immunoglobulins (recipient antibody) in which residues from a CDR of the recipient are substituted by residues from a CDR of a non-feline species (donor antibody) such as mouse, rat, or rabbit having the desired specificity, affinity, and capacity. In some instances, Fv framework region (FR) residues of the feline immunoglobulin are replaced by corresponding non-feline residues. Furthermore, the felinized antibody can comprise residues that are found neither in the recipient antibody nor in the imported CDR or framework sequences, but are included to further refine and optimize antibody performance.

An “equinized antibody” means an antibody in which at least one amino acid in a portion of a non-equine variable region has been replaced with the corresponding amino acid from an equine variable region. In some embodiments, an equinized antibody comprises at least one equine constant region (e.g., a γ constant region, an α constant region, a δ constant region, an ε constant region, a μ constant region, or etc.) or fragment thereof. In some embodiments, an equinized antibody is an antibody fragment, such as Fab, scFv, (Fab′)2, etc. The term “equinized” also denotes forms of non-equine (for example, murine) antibodies that are chimeric immunoglobulins, immunoglobulin chains, or fragments thereof (such as Fv, Fab, Fab′, F(ab′)2 or other antigen-binding sequences of antibodies) that contain minimal sequence of non-equine immunoglobulin. Equinized antibodies can include equine immunoglobulins (recipient antibody) in which residues from a CDR of the recipient are substituted by residues from a CDR of a non-equine species (donor antibody) such as mouse, rat, or rabbit having the desired specificity, affinity, and capacity. In some instances, Fv framework region (FR) residues of the equine immunoglobulin are replaced by corresponding non-equine residues. Furthermore, the equinized antibody can comprise residues that are found neither in the recipient antibody nor in the imported CDR or framework sequences, but are included to further refine and optimize antibody performance.

In some embodiments, at least one amino acid residue in a portion of a mouse variable heavy chain or a mouse variable light chain has been replaced with the corresponding amino acid from an equine variable region. In some embodiments, the modified chain is fused to an equine constant heavy chain or a canine constant light chain.

The term “IgX Fc” means the Fc region is derived from a particular antibody isotype (e.g., IgG, IgA, IgD, IgE, IgM, etc.), where “X” denotes the antibody isotype. Thus, “IgG Fc” denotes the Fc region of a γ chain, “IgA Fc” denotes the Fc region of an α chain, “IgD Fc” denotes the Fc region of a δ chain, “IgE Fc” denotes the Fc region of an ε chain, “IgM Fc” denotes the Fc region of a μ chain, etc. In some embodiments, the IgG Fc region comprises CH1, hinge, CH2, CH3, and CL1. “IgX-N-Fc” denotes that the Fc region is derived from a particular subclass of antibody isotype (such as canine IgG subclass A, B, C, or D; feline IgG subclass 1a, 1b, or 2; or equine IgG subclass IgG1, IgG2, IgG3, IgG4, IgG5, IgG6, or IgG7, etc.), where “N” denotes the subclass. In some embodiments, IgX Fc or IgX-N-Fc regions are derived from a companion animal, such as a dog, a cat, or a horse. In some embodiments, IgG Fc regions are isolated from canine γ heavy chains, such as IgG-A, IgG-B, IgG-C, or IgG-D. In some instances, IgG Fc regions are isolated from feline γ heavy chains, such as IgG1a, IgG1 b, or IgG2. Antibodies comprising an Fc region of IgG-A, IgG-B, IgG-C, or IgG-D may provide for higher expression levels in recombination production systems.

As used herein, “percent (%) amino acid sequence identity” and “homology” with respect to a peptide, polypeptide, or antibody sequence are defined as the percentage of amino acid residues in a candidate sequence that are identical with the amino acid residues in the specific peptide or polypeptide sequence, after aligning the sequences and introducing gaps, if necessary to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALINE (DNASTAR) software. Those skilled in the art can determine appropriate parameters for measuring alignment, including any algorithms needed to achieve maximal alignment over the full length of sequences being compared.

An amino acid substitution may include but is not limited to the replacement of one amino acid in a polypeptide with another amino acid. Exemplary substitutions are shown in Table 2. Amino acid substitutions may be introduced into an antibody of interest and the products screened for a desired activity, for example, retained/improved antigen binding, decreased immunogenicity, or improved ADCC or CDC.

TABLE 2

| Original Residue | Exemplary Substitutions |
|---|---|
| Ala (A) | Val; Leu; Ile |
| Arg (R) | Lys; Gln; Asn |
| Asn (N) | Gln; His; Asp; Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; |
| | Norleucine |
| Leu (L) | Norleucine; Ile; Val; Met; Ala; |
| | Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; |
| | Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes with another class.

In some embodiments, an anti-IL17A antibody comprises a heavy chain and a light chain, wherein:
(a) (i) the heavy chain comprises a CDR-H1 sequence having at least 85%, at least 90%, at least 95, or at least 98% sequence identity to the amino acid sequence of the amino acid sequence of SEQ ID NO: 1, a CDR H2 sequence having at least 85%, at least 90%, at least 95, or at least 98% sequence identity to the amino acid sequence of the amino acid sequence of SEQ ID NO: 2, and a CDR-H3 sequence having at least 85%, at least 90%, at least 95, or at least 98% sequence identity to the amino acid sequence of the amino acid sequence of SEQ ID NO: 3, and
   (ii) the light chain comprises a CDR-L1 sequence having at least 85%, at least 90%, at least 95, or at least 98% sequence identity to the amino acid sequence of the amino acid sequence of SEQ ID NO: 8, a CDR-L2 sequence having at least 85%, at least 90%, at least 95, or at least 98% sequence identity to the amino acid sequence of the amino acid sequence of SEQ ID NO: 9 or SEQ ID NO: 108, and a CDR-L3 sequence having at least 85%, at least 90%, at least 95, or at least 98% sequence identity to the amino acid sequence of the amino acid sequence of SEQ ID NO: 10; or
(b) (i) the heavy chain comprises a CDR-H1 sequence having at least 85%, at least 90%, at least 95, or at least 98% sequence identity to the amino acid sequence of the amino acid sequence of SEQ ID NO: 52, a CDR H2 sequence having at least 85%, at least 90%, at least 95, or at least 98% sequence identity to the amino acid sequence of the amino acid sequence of SEQ ID NO: 53 or SEQ ID NO: 109, and a CDR-H3 sequence having at least 85%, at least 90%, at least 95, or at least 98% sequence identity to the amino acid sequence of the amino acid sequence of SEQ ID SEQ ID NO: 54, and
   (ii) the light chain comprises a CDR-L1 sequence having at least 85%, at least 90%, at least 95, or at least 98% sequence identity to the amino acid sequence of the amino acid sequence of SEQ ID NO: 59 or SEQ ID NO: 111, a CDR-L2 sequence having at least 85%, at least 90%, at least 95, or at least 98% sequence identity to the amino acid sequence of the amino acid sequence of SEQ ID NO: 60 or SEQ ID NO: 112, and a CDR-L3 sequence having at least 85%, at least 90%, at least 95, or at least 98% sequence identity to the amino acid sequence of the amino acid sequence of SEQ ID NO: 61; or
(c) (i) the heavy chain comprises a CDR-H1 sequence having at least 85%, at least 90%, at least 95, or at least 98% sequence identity to the amino acid sequence of the amino acid sequence of SEQ ID NO: 66, a CDR H2 sequence having at least 85%, at least 90%, at least 95, or at least 98% sequence identity to the amino acid sequence of the amino acid sequence of SEQ ID NO: 67 or SEQ ID NO: 114, and a CDR-H3 sequence having at least 85%, at least 90%, at least 95, or at least 98% sequence identity to the amino acid sequence of the amino acid sequence of SEQ ID NO: 68, and
   (ii) the light chain comprises a CDR-L1 sequence having at least 85%, at least 90%, at least 95, or at least 98% sequence identity to the amino acid sequence of the amino acid sequence of SEQ ID NO: 73 or SEQ ID NO: 116, a CDR-L2 sequence having at least 85%, at least 90%, at least 95, or at least 98% sequence identity to the amino acid sequence of the amino acid sequence of SEQ ID NO: 74 or SEQ ID NO: 117; and a CDR-L3 sequence having at least 85%, at least 90%, at least 95, or at least 98% sequence identity to the amino acid sequence of the amino acid sequence of SEQ ID NO: 75; or
(d) (i) the heavy chain comprises a CDR-H1 sequence having at least 85%, at least 90%, at least 95, or at least 98% sequence identity to the amino acid sequence of the amino acid sequence of SEQ ID NO: 80, a CDR H2 sequence having at least 85%, at least 90%, at least 95, or at least 98% sequence identity to the amino acid sequence of the amino acid sequence of SEQ ID NO: 81 or SEQ ID NO: 119, and a CDR-H3 sequence having at least 85%, at least 90%, at least 95, or at least 98% sequence identity to the amino acid sequence of the amino acid sequence of SEQ ID NO: 82, and
   (ii) the light chain comprises a CDR-L1 sequence having at least 85%, at least 90%, at least 95, or at least 98% sequence identity to the amino acid sequence of the amino acid sequence of SEQ ID NO: 87 or SEQ ID NO: 121, a CDR-L2 sequence having at least 85%, at least 90%, at least 95, or at least 98% sequence identity to the amino acid sequence of the amino acid sequence of SEQ ID NO: 88 or SEQ ID NO: 122, and a CDR-L3 sequence having at least 85%, at least 90%, at least 95, or at least 98% sequence identity to the amino acid sequence of the amino acid sequence of SEQ ID NO: 89.

In some embodiments, an anti-IL17A antibody comprises a heavy chain and a light chain, wherein:
(a) (i) a variable light chain sequence having at least 85%, at least 90%, at least 95%, or at least 98% sequence identity to the amino acid sequence of SEQ ID NO: 24; (ii) a variable heavy chain sequence having at least 85%, at least 90%, at least 95%, or at least 98% sequence identity to the amino acid sequence of SEQ ID NO: 25; or (iii) a variable light chain sequence as in (i) and a variable heavy chain sequence as in (ii); or
(b) (i) a variable light chain sequence having at least 85%, at least 90%, at least 95%, or at least 98% sequence identity to the amino acid sequence of SEQ ID NO: 16; (ii) a variable heavy chain sequence having at least 85%, at least 90%, at least 95%, or at least 98% sequence identity to the amino acid sequence of SEQ ID NO: 15; or (iii) a variable light chain sequence as in (i) and a variable heavy chain sequence as in (ii); or
(c) (i) a variable light chain sequence having at least 85%, at least 90%, at least 95%, or at least 98% sequence identity to the amino acid sequence of SEQ ID NO: 34; (ii) a variable heavy chain sequence having at least 85%, at least 90%, at least 95%, or at least 98% sequence identity to the amino acid sequence of SEQ ID NO: 35; or (iii) a variable light chain sequence as in (i) and a variable heavy chain sequence as in (ii); or
(d) (i) a variable light chain sequence having at least 85%, at least 90%, at least 95%, or at least 98% sequence identity to the amino acid sequence of SEQ ID NO: 38; (ii) a variable heavy chain sequence having at least 85%, at least 90%, at least 95%, or at least 98% sequence identity to the amino acid sequence of SEQ ID NO: 39; or (iii) a variable light chain sequence as in (i) and a variable heavy chain sequence as in (ii); or
(e) (i) a variable light chain sequence having at least 85%, at least 90%, at least 95%, or at least 98% sequence identity to the amino acid sequence of SEQ ID NO: 36; (ii) a variable heavy chain sequence having at least 85%, at least 90%, at least 95%, or at least 98% sequence identity to the amino acid sequence of SEQ ID NO: 37; or (iii) a variable light chain sequence as in (i) and a variable heavy chain sequence as in (ii).

### Exemplary IL17Ra Polypeptides

“IL17Ra,” as used herein, is a polypeptide comprising the entirety or a fragment of IL17A receptor that binds to IL17A.

For example, “IL7Ra” refers to an IL17Ra polypeptide from any vertebrate source, including mammals such as primates (e.g., humans and cynomolgus monkeys), rodents (e.g., mice and rats), and companion animals (e.g., dogs, cats, and equine), unless otherwise indicated. In some embodiments, IL17Ra is an extracellular domain fragment that binds IL17A. In some such embodiments, the IL17Ra may be referred to as an IL17Ra extracellular domain (ECD). In some embodiments, IL4R comprises the amino acid sequence of SEQ ID NO: 32, SEQ ID NO: 33, SEQ ID NO: SEQ ID NO: 94, SEQ ID NO: 97, SEQ ID NO: 98, or SEQ ID NO: 99.

An “extracellular domain” (“ECD”) is the portion of a polypeptide that extends beyond the transmembrane domain into the extracellular space. The term “extracellular domain,” as used herein, may comprise a complete extracellular domain or may comprise a truncated extracellular domain missing one or more amino acids, that binds to its ligand. The composition of the extracellular domain may depend on the algorithm used to determine which amino acids are in the membrane. Different algorithms may predict, and different systems may express, different extracellular domains for a given protein.

An extracellular domain of an IL17Ra polypeptide may comprise a complete extracellular domain or a truncated extracellular domain of IL17Ra that binds IL17A. As used herein, the terms “extracellular domain of an IL17Ra polypeptide,” “IL17Ra ECD,” and similar terms refer to an IL17Ra polypeptide that does not comprise a transmembrane domain or cytoplasmic domain, even if the term follows an open transitional word, such as “comprising,” “comprises,” and the like. In some embodiments, an extracellular domain of an IL17Ra polypeptide is an extracellular domain of an IL17Ra polypeptide derived from a companion species animal or a human. For example, in some embodiments, an extracellular domain of an IL17Ra polypeptide is derived from canine IL17Ra, feline IL17Ra or equine IL17Ra. In some embodiments, an extracellular domain of an IL17Ra polypeptide comprises the amino acid sequence of SEQ ID NO: 33, SEQ ID NO: 94, SEQ ID NO: 97, SEQ ID NO: 98, SEQ ID NO: 99, or any fragment thereof.

A polypeptide of the invention may comprise an extracellular domain of an IL17Ra polypeptide, wherein the polypeptides are derived from a human or a companion animal species. For example, a polypeptide may comprise an extracellular domain of an IL17Ra polypeptide from a human, a dog, a cat, or a horse.

Polypeptides comprising an extracellular domain of an IL17Ra polypeptide can function as decoy receptors for trapping IL17A and inhibiting its interaction with IL17Ra on cell surfaces. Decoy receptors, such as those of the invention, recognize their ligands with high affinity and specificity but are structurally incapable of signaling. They compete with wild-type receptors for ligand binding and participate in ligand/receptor interactions, thus modulating the activity of or the number of functioning receptors and/or the cellular activity downstream from the receptors. Decoy receptors can act as molecular traps for agonist ligands and thereby inhibit ligand-induced receptor activation.

“Wild-type” refers to a non-mutated version of a polypeptide that occurs in nature, or a fragment thereof. A wild-type polypeptide may be produced recombinantly. A “wild-type IL17Ra ECD” refers to a protein having an amino acid sequence that is identical to the same portion of an extracellular domain of an IL17Ra that occurs in nature.

A “variant,” as used herein is a polypeptide that differs from a reference polypeptide by single or multiple amino acid substitutions, deletions, and/or additions and substantially retains at least one biological activity of the reference polypeptide.

A “biologically active” entity, or an entity having “biological activity,” is an entity having any function related to or associated with a metabolic or physiological process, and/or having structural, regulatory, or biochemical functions of a naturally-occurring molecule. Biologically active polynucleotide fragments are those exhibiting similar activity, but not necessarily identical, to an activity of a polynucleotide of the present invention. A biologically active polypeptide or fragment thereof includes one that can participate in a biological reaction, including, but not limited to, a ligand-receptor interaction or antigen-antibody binding. The biological activity can include an improved desired activity, or a decreased undesirable activity. An entity may demonstrate biological activity when it participates in a molecular interaction with another molecule, such as hybridization, when it has therapeutic value in alleviating a disease condition, when it has prophylactic value in inducing an immune response, when it has diagnostic and/or prognostic value in determining the presence of a molecule, such as a biologically active fragment of a polynucleotide that may be detected as unique for the polynucleotide molecule, and when it can be used as a primer in a polymerase chain reaction (PCR).

In some embodiments, a variant has at least about 50% amino acid sequence identity, at least about 60% amino acid sequence identity, at least about 65% amino acid sequence identity, at least about 70% amino acid sequence identity, at least about 75% amino acid sequence identity, at least about 80% amino acid sequence identity, at least about 85% amino acid sequence identity, at least about 90% amino acid sequence identity, at least about 95% amino acid sequence identity, or at least 99% amino acid sequence identity with the native sequence polypeptide.

In some embodiments, an IL17Ra ECD polypeptide has at least 85%, at least 90%, at least 95%, at least 98% sequence identity to the amino acid sequence of SEQ ID NO: 33, SEQ ID NO: 94, SEQ ID NO: 97, SEQ ID NO: 98, or SEQ ID NO: 99.

### Exemplary IgG Fc Variants

Novel IgG Fc variants are provided, for example IgG Fc variants with altered binding affinity (e.g., reduced binding affinity) to C1q and CD16.

A“fragment crystallizable polypeptide” or “Fc polypeptide” is the portion of an antibody molecule that interacts with effector molecules and cells. It comprises the C terminal portions of the immunoglobulin heavy chains. As used herein, an Fc polypeptide includes fragments of the Fc domain having one or more biological activity of an entire Fc polypeptide.

An “IgG Fc variant” as used herein is an IgG Fc polypeptide that differs from a reference IgG Fc polypeptide by single or multiple amino acid substitutions, deletions, and/or additions and substantially retains at least one biological activity of the reference IgG Fc polypeptide.

In some embodiments, an IgG Fc variant may have reduced complement fixation and/or antibody-dependent cellular cytotoxicity (ADCC) induction. In some embodiments, an IgG Fc variant has reduced binding affinity to C1q and/or CD16.

In some embodiments, an IgG Fc variant polypeptide comprises an amino acid substitution at a position corresponding to position 110 of SEQ ID NO: 45 or at a position corresponding to position 108 of SEQ ID NO: 46. In some embodiments, an IgG Fc variant polypeptide comprises an amino acid substitution at a position corresponding to position 55 of SEQ ID NO: 45 or at a position corresponding to position 43 of SEQ ID NO: 46. In some embodiments, an IgG Fc variant polypeptide comprises an amino acid substitution at a position corresponding to position 114 of SEQ ID NO: 45 or at a position corresponding to position 112 of SEQ ID NO: 46. In some embodiments, an IgG Fc variant polypeptide comprises an amino acid substitution at a position corresponding to position 115 at SEQ ID NO: 45 or at a position corresponding to position 113 of SEQ ID NO: 46.

In some embodiments, an IgG Fc variant polypeptide comprises an amino acid substitution at position 110 of SEQ ID NO: 45 or at position 108 of SEQ ID NO: 46. In some embodiments, an IgG Fc variant polypeptide comprises an amino acid substitution at position 55 of SEQ ID NO: 45 or at position 43 of SEQ ID NO: 46. In some embodiments, an IgG Fc variant polypeptide comprises an amino acid substitution at position 114 of SEQ ID NO: 45 or at position 112 of SEQ ID NO: 46. In some embodiments, an IgG Fc variant polypeptide comprises an amino acid substitution at position 115 at SEQ ID NO: 45 or at position 113 of SEQ ID NO: 46.

In some embodiments, an IgG Fc variant polypeptide comprises an arginine at position 110 of SEQ ID NO: 45 or at position 108 of SEQ ID NO: 46. In some embodiments, an IgG Fc variant polypeptide comprises a glycine at position 55 of SEQ ID NO: 45 or at position 43 of SEQ ID NO: 46. In some embodiments, an IgG Fc variant polypeptide comprises an isoleucine at position 114 of SEQ ID NO: 45 or at position 112 of SEQ ID NO: 46. In some embodiments, an IgG Fc variant polypeptide comprises a glycine at position 115 at SEQ ID NO: 45 or at position 113 of SEQ ID NO: 46.

In some embodiments, an IgG Fc variant polypeptide comprises the amino acid sequence of SEQ ID NO: 47, SEQ ID NO: 48, SEQ ID NO: 49, or SEQ ID NO: 50. In some embodiments, the polypeptide comprising an IgG Fc variant polypeptide comprises the amino acid sequence of SEQ ID NO: 96.

An “amino acid derivative,” as used herein, refers to any amino acid, modified amino acid, and/or amino acid analogue, that is not one of the 20 common natural amino acids found in humans. Exemplary amino acid derivatives include natural amino acids not found in humans (e.g., seleno cysteine and pyrrolysine, which may be found in some microorganisms) and unnatural amino acids. Exemplary amino acid derivatives, include, but are not limited to, amino acid derivatives commercially available through chemical product manufacturers and distributors (e.g., sigmaaldrich.com, accessed on May 6, 2017, which is incorporated herein by reference). One or more amino acid derivative maybe incorporated into a polypeptide at a specific location using translation systems that utilize host cells, orthogonal aminoacyl-tRNA synthetases derived from eubacterial synthetases, orthogonal tRNAs, and an amino acid derivative. For further descriptions, see, e.g., U.S. Pat. No. 9,624,485.

In some embodiments, an IgG Fc variant polypeptide or other polypeptide described herein comprises an amino acid substitution with an amino acid derivative.

A “fusion partner,” as used herein, refers to an additional component of a polypeptide, such as albumin, an albumin binding fragment, or a fragment of an immunoglobulin molecule. A fusion partner may comprise an oligomerization domain such as an Fc domain of a heavy chain immunoglobulin.

In some embodiments, an IgG Fc variant polypeptide is a fusion partner to an IL17A antibody or IL17Ra ECD polypeptide as described herein. In some embodiments, a polypeptide comprises an IgG Fc variant polypeptide and an IL17A antibody and/or IL17Ra ECD polypeptide as described herein. In some embodiments, a polypeptide comprises an IgG Fc variant polypeptide and another polypeptide. In some embodiments, a polypeptide comprises the amino acid sequence of SEQ ID NO: 96.

### Exemplary Target Binding Affinity

The term “affinity” means the strength of the sum total of noncovalent interactions between a single binding site of a molecule (for example, an antibody) and its binding partner (for example, an antigen). The affinity of a molecule X for its partner Y can generally be represented by the dissociation constant (Kd). Affinity can be measured by common methods known in the art, such as, for example, immunoblot, ELISA KD, KinExA, biolayer interferometry (BLI), or surface plasmon resonance devices.

The terms “Kd,” “KD,” “Kd” or “Kd value” as used interchangeably to refer to the equilibrium dissociation constant of an antibody-antigen interaction. In some embodiments, the Kd of the antibody is measured by using biolayer interferometry assays using a biosensor, such as an OCTET System (Pall ForteBio LLC, Fremont, CA) according to the supplier's instructions. Briefly, biotinylated antigen is bound to the sensor tip and the association of antibody is monitored for ninety seconds and the dissociation is monitored for 600 seconds. The buffer for dilutions and binding steps is 20 mM phosphate, 150 mM NaCl, pH 7.2. A buffer only blank curve is subtracted to correct for any drift. The data are fit to a 1:1 binding model using FORTEBIO data analysis software to determine association rate constant (kon), dissociation rate constant (koff), and the Kd. The equilibrium dissociation constant (Kd) is calculated as the ratio of koff/kon. The term “kon” refers to the rate constant for association of an antibody to an antigen and the term “koff” refers to the rate constant for dissociation of an antibody from the antibody/antigen complex.

The term “binds” to an antigen or epitope is a term that is well understood in the art, and methods to determine such binding are also well known in the art. A molecule is said to exhibit “binding” if it reacts, associates with, or has affinity for a particular cell or substance and the reaction, association, or affinity is detectable by one or more methods known in the art, such as, for example, immunoblot, ELISA KD, KinExA, biolayer interferometry (BLI), surface plasmon resonance devices, or etc.

“Surface plasmon resonance” denotes an optical phenomenon that allows for the analysis of real-time biospecific interactions by detection of alterations in protein concentrations within a biosensor matrix, for example using the BIACORE system (BIAcore International AB, a GE Healthcare company, Uppsala, Sweden and Piscataway, N.J.). For further descriptions, see Jonsson et al. (1993) Ann. Biol. Clin. 51: 19-26.

“Biolayer interferometry” refers to an optical analytical technique that analyzes the interference pattern of light reflected from a layer of immobilized protein on a biosensor tip and an internal reference layer. Changes in the number of molecules bound to the biosensor tip cause shifts in the interference pattern that can be measured in real-time. A nonlimiting exemplary device for biolayer interferometry is an OCTET system (Pall ForteBio LLC). See, e.g., Abdiche et al., 2008, Anal. Biochem. 377: 209-277.

In some embodiments, an anti-IL17A antibody or an IL17Ra ECD binds to IL17A, such as canine IL17A, feline IL17A, or equine IL17A with a dissociation constant (Kd) of less than 5×10-6 M, less than 1×10-6 M, less than 5×10-7 M, less than 1×10-7 M, less than 5×10-8 M, less than 1×10-8 M, less than 5×10-9 M, less than 1×10-9 M, less than 5×10-10 M, less than 1×10-10 M, less than 5×10 11 M, less than 1×10 11 M, less than 5×10-12 M, or less than 1×10-12 M, as measured by biolayer interferometry. In some embodiments, an anti-IL17A antibody or an IL17Ra ECD binds to canine IL17A, feline IL17A, or equine IL17A with a Kd of between 5×10-6 M and 1×10-6 M, between 5×10-6 M and 5×10-7 M, between 5×10-6 M and 1×10-7 M, between 5×10-6 M and 5×10-8 M, 5×10-6 M and 1×10-8 M, between 5×10-6 M and 5×10-9 M, between 5×10-6 M and 1×10-9 M, between 5×10-6 M and 5×10-10 M, between 5×10-6 M and 1×10-10 M, between 5×10-6 M and 5×10-11 M, between 5×10-6 M and 1×10-11 M, between 5×10-6 M and 5×10-12 M, between 5×10-6 M and 1×10-12 M, between 1×10-6 M and 5×10-7 M, between 1×10-6 M and 1×10-7 M, between 1×10-6 M and 5×10-8 M, 1×10-6 M and 1×10-8 M, between 1×10-6 M and 5×10-9 M, between 1×10-6 M and 1×10-9 M, between 1×10-6 M and 5×10-10 M, between 1×10-6 M and 1×10-10 M, between 1×10-6 M and 5×10-11 M, between 1×10-6 M and 1×10-11 M, between 1×10-6 M and 5×10-12 M, between 1×10-6 M and 1×10-12 M, between 5×10-7 M and 1×10-7 M, between 5×10-7 M and 5×10-8 M, 5×10-7 M and 1×10-8 M, between 5×10-7 M and 5×10-9 M, between 5×10-7 M and 1×10-9 M, between 5×10-7 M and 5×10-10 M, between 5×10-7 M and 1×10-10 M, between 5×10-7 M and 5×10-11 M, between 5×10-7 M and 1×10-11 M, between 5×10-7 M and 5×10-12 M, between 5×10-7 M and 1×10-12 M, between 1×10-7 M and 5×10-8 M, 1×10-7 M and 1×10-8 M, between 1×10-7 M and 5×10-9 M, between 1×10-7 M and 1×10-9 M, between 1×10-7 M and 5×10-10 M, between 1×10-7 M and 1×10-10 M, between 1×10-7 M and 5×10-11 M, between 1×10-7 M and 1×10-11 M, between 1×10-7 M and 5×10-12 M, between 1×10-7 M and 1×10-12 M, between 5×10-8 M and 1×10-8 M, between 5×10-8 M and 5×10-9 M, between 5×10-8 M and 1×10-9 M, between 5×10 8 M and 5×10-10 M, between 5×10-8 M and 1×10-10 M, between 5×10-8 M and 5×10-11 M, between 5×10 8 M and 1×10-11 M, between 5×10-8 M and 5×10-12 M, between 5×10-8 M and 1×10-12 M, 1×10-8 M and 5×10-9 M, between 1×10-8 M and 1×10-9 M, between 1×10-8 M and 5×10-10 M, between 1×10-8 M and 1×10-10 M, between 1×10-8 M and 5×10-11 M, between 1×10 8 M and 1×10-11 M, between 1×10-8 M and 5×10-12 M, between 1×10-8 M and 1×10-12 M, between 5×10-9 M and 1×10-9 M, between 5×10-9 M and 5×10-10 M, between 5×10-9 M and 1×10-10 M, between 5×10 9 M and 5×10-11 M, between 5×10 9 M and 1×10-11 M, between 5×10-9 M and 5×10-12 M, between 5×10-9 M and 1×10-12 M, between 1×10-9 M and 5×10-10 M, between 1×10-9 M and 1×10-10 M, between 1×10-9 M and 5×10-11 M, between 1×10-9 M and 1×10-11 M, between 1×10-9 M and 5×10-12 M, between 1×10-9 M and 1×10-12 M, between 5×10 10 M and 1×10-10 M, between 5×10-10 M and 5×10-11 M, between, 1×10-10 M and 5×10-11 M, 1×10-10 M and 1×10-11 M, between 1×10-10 M and 5×10-12 M, between 1×10-10 M and 1×10 12 M, between 5×10-11 M and 1×10-12 M, between 5×10-11 M and 5×10-12 M, between 5×10 11 M and 1×10-12 M, between 1×10-11 M and 5×10 12 M, or between 1×10-11 M and 1×10-12 M, as measured by biolayer interferometry. In some embodiments, an anti-IL17A antibody or IL17Ra ECD binds to canine IL17A, feline IL17A, or equine IL17A, as determined by immunoblot analysis.

In some embodiments, an anti-IL17A antibody is provided that competes with an anti-IL17A antibody described herein (such as Clone C, Clone A, Clone D, or Clone E) for binding to IL17A. In some embodiments, an antibody that competes with binding with any of the antibodies provided herein can be made or used. In some embodiments, an anti-IL17A antibody is provided that competes with monoclonal Clone C, Clone A, Clone D, or Clone E antibody in binding to canine IL17A, feline IL17A, or equine IL17A.

The term “IL17A signaling function” refers to any one of or combination of the downstream activities that occurs when IL17A binds its receptor or receptor complex.

In some embodiments, the IL17A signaling function comprises activation of NFκB, MAPKs and/or C/EBPs to induce cytokines, chemokines, and/or host defense to microbial infection. In some embodiments, the IL17A signaling function comprises activating production of IL6.

To “reduce” or “inhibit” means to decrease, reduce, or arrest an activity, function, or amount as compared to a reference. In some embodiments, by “reduce” or “inhibit” is meant the ability to cause an overall decrease of 20% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control dose (such as a placebo) over the same period of time.

To “increase” or“stimulate” means to increase, improve, or augment an activity, function, or amount as compared to a reference. In some embodiments, by“reduce” or “inhibit” is meant the ability to cause an overall increase of 20% or greater. In some embodiments, by “increase” or “stimulate” is meant the ability to cause an overall increase of 50% or greater. In some embodiments, by “increase” or “stimulate” is meant the ability to cause an overall increase of 75%, 85%, 90%, 95%, or greater. In some embodiments, the amount noted above is stimulated or increased over a period of time, relative to a control dose (such as a placebo) over the same period of time.

A “reference” as used herein, refers to any sample, standard, or level that is used for comparison purposes. A reference may be obtained from a healthy or non-diseased sample. In some examples, a reference is obtained from a non-diseased or non-treated sample of a companion animal. In some examples, a reference is obtained from one or more healthy animals of a particular species, which are not the animal being tested or treated.

The term “substantially reduced,” as used herein, denotes a sufficiently high degree of reduction between a numeric value and a reference numeric value such that one of skill in the art would consider the difference between the two values to be of statistical significance within the context of the biological characteristic measured by said values. In some embodiments, the substantially reduced numeric values is reduced by greater than about any one of 10%, 15% 20%, 25%, 30%, 35%, 40%, 45%, 50%, 60%, 70%, 80%, 90%, or 100% compared to the reference value.

In some embodiments, an IL17A antibody or an IL17Ra ECD polypeptide may reduce IL17A signaling function in a companion animal species by at least 10%, at least 15%, at least 20%, at least 25%, at least 30%, at least 35%, at least 40%, at least 45%, at least 50%, at least 60%, at least 70%, at least 80%, at least 90%, or 100% compared to IL17A signaling function in the absence of the IL17A antibody or IL17Ra ECD polypeptide, as measured by a reduction in IL6 production. In some embodiments, the reduction in IL17A signaling function or the reduction in IL6 production is between 10% and 15%, between 10% and 20%, between 10% and 25%, between 10% and 30%, between 10% and 35%, between 10% and 40%, between 10% and 45%, between 10% and 50%, between 10% and 60%, between 10% and 70%, between 10% and 80%, between 10% and 90%, between 10% and 100%, between 15% and 20%, between 15% and 25%, between 15% and 30%, between 15% and 35%, between 15% and 40%, between 15% and 45%, between 15% and 50%, between 15% and 60%, between 15% and 70%, between 15% and 80%, between 15% and 90%, between 15% and 100%, between 20% and 25%, between 20% and 30%, between 20% and 35%, between 20% and 40%, between 20% and 45%, between 20% and 50%, between 20% and 60%, between 20% and 70%, between 20% and 80%, between 20% and 90%, between 20% and 100%, between 25% and 30%, between 25% and 35%, between 25% and 40%, between 25% and 45%, between 25% and 50%, between 25% and 60%, between 25% and 70%, between 25% and 80%, between 25% and 90%, between 25% and 100%, between 30% and 35%, between 30% and 40%, between 30% and 45%, between 30% and 50%, between 30% and 60%, between 30% and 70%, between 30% and 80%, between 30% and 90%, between 30% and 100%, between 35% and 40%, between 35% and 45%, between 35% and 50%, between 35% and 60%, between 35% and 70%, between 35% and 80%, between 35% and 90%, between 35% and 100%, between 40% and 45%, between 40% and 50%, between 40% and 60%, between 40% and 70%, between 40% and 80%, between 40% and 90%, between 40% and 100%, between 45% and 50%, between 45% and 60%, between 45% and 70%, between 45% and 80%, between 45% and 90%, between 45% and 100%, between 50% and 60%, between 50% and 70%, between 50% and 80%, between 50% and 90%, between 50% and 100%, between 60% and 70%, between 60% and 80%, between 60% and 90%, between 60% and 100%, between 70% and 80%, between 70% and 90%, between 70% and 100%, between 80% and 90%, between 80% and 100%, or between 90% and 100%.

### Exemplary Antibody and Polypeptide Expression and Production

Polynucleotide sequences that encode all or part of a polypeptide with or without a signal sequence are provided. If a homologous signal sequence (e.g., a signal sequence of native IL-17Ra) is not used in the construction of the nucleic acid molecule, then another signal sequence may be used, for example, any one of the signal sequences described in PCT US06/02951.

Typically, nucleotide sequence encoding the polypeptide of interest, such as an IL17A antibody, an IL17Ra ECD polypeptide, an IgG Fc variant polypeptide, or a polypeptide comprising such, is inserted into an expression vector, suitable for expression in a selected host cell.

The term “vector” is used to describe a polynucleotide that can be engineered to contain a cloned polynucleotide or polynucleotides that can be propagated in a host cell. A vector can include one or more of the following elements: an origin of replication, one or more regulatory sequences (such as, for example, promoters or enhancers) that regulate the expression of the polypeptide of interest, or one or more selectable marker genes (such as, for example, antibiotic resistance genes and genes that can be used in colorimetric assays, for example, p galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NS0 cells, PER.C6© cells (Crucell), 293 cells, and CHO cells, and their derivatives, such as 293-6E, DG44, CHO-S, and CHO-K cells. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) encoding an amino acid sequence(s) provided herein.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated.” In some embodiments, an IL17A antibody, an IL17Ra ECD polypeptide, an IgG Fc variant polypeptide, or a polypeptide comprising such, is purified using chromatography, such as size exclusion chromatography, ion exchange chromatography, protein A column chromatography, hydrophobic interaction chromatography, and CHT chromatography.

### Exemplary Pharmaceutical Compositions

The terms “pharmaceutical formulation” and “pharmaceutical composition” refer to a preparation which is in such form as to permit the biological activity of the active ingredient(s) to be effective, and which contains no additional components that are unacceptably toxic to a subject to which the formulation would be administered.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. Examples of pharmaceutically acceptable carriers include alumina; aluminum stearate; lecithin; serum proteins, such as human serum albumin, canine or other animal albumin; buffers such as phosphate, citrate, tromethamine or HEPES buffers; glycine; sorbic acid; potassium sorbate; partial glyceride mixtures of saturated vegetable fatty acids; water; salts or electrolytes, such as protamine sulfate, disodium hydrogen phosphate, potassium hydrogen phosphate, sodium chloride, zinc salts, colloidal silica, or magnesium trisilicate; polyvinyl pyrrolidone, cellulose-based substances; polyethylene glycol; sucrose; mannitol; or amino acids including, but not limited to, arginine.

The pharmaceutical composition can be stored in lyophilized form. Thus, in some embodiments, the preparation process includes a lyophilization step. The lyophilized composition may then be reformulated, typically as an aqueous composition suitable for parenteral administration, prior to administration to the dog, cat, or horse. In other embodiments, particularly where the antibody is highly stable to thermal and oxidative denaturation, the pharmaceutical composition can be stored as a liquid, i.e., as an aqueous composition, which may be administered directly, or with appropriate dilution, to the dog, cat, or horse. A lyophilized composition can be reconstituted with sterile Water for Injection (WFI). Bacteriostatic reagents, such benzyl alcohol, may be included. Thus, the invention provides pharmaceutical compositions in solid or liquid form.

The pH of the pharmaceutical compositions may be in the range of from about pH 5 to about pH 8, when administered. The compositions of the invention are sterile if they are to be used for therapeutic purposes. Sterility can be achieved by any of several means known in the art, including by filtration through sterile filtration membranes (e.g., 0.2 micron membranes). Sterility may be maintained with or without anti-bacterial agents.

### Certain Uses of Antibodies and Pharmaceutical Compositions

The antibodies or pharmaceutical compositions comprising the antibodies of the invention may be useful for treating an IL-17A-induced condition. As used herein, an “IL17A-induced condition” means a disease associated with, caused by, or characterized by, elevated levels or altered gradients of IL17A concentration. Such IL17A-induced conditions include, but are not limited to, proinflammatory functions, such as plaque psoriasis, psoriatic arthritis, rheumatoid arthritis, airway inflammation, asthma, osteoarthritis, inflammatory bowel disorder, Crohn's disease, ankylosing spondylitis, atopic dermatitis, degenerative myelopathy, multiple sclerosis, and uveitis. An IL17A-induced condition may be exhibited in a human or a companion animal, including, but not limited to, canine, feline, or equine.

As used herein, “treatment” is an approach for obtaining beneficial or desired clinical results. “Treatment” as used herein, covers any administration or application of a therapeutic for disease in a mammal, including a companion animal. For purposes of this disclosure, beneficial or desired clinical results include, but are not limited to, any one or more of: alleviation of one or more symptoms, diminishment of extent of disease, preventing or delaying spread of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

In some embodiments, an anti-IL17A antibody, an IL17Ra ECD polypeptide, an IgG Fc variant polypeptide, or a polypeptide or pharmaceutical composition comprising such can be utilized in accordance with the methods herein to treat IL17A-induced conditions. In some embodiments, an antibody, polypeptide, or pharmaceutical composition is administered to a human or to a companion animal, such as a canine, a feline, or equine, to treat an IL17A-induced condition.

A “therapeutically effective amount” of a substance/molecule, agonist or antagonist may vary according to factors such as the type of disease to be treated, the disease state, the severity and course of the disease, the type of therapeutic purpose, any previous therapy, the clinical history, the response to prior treatment, the discretion of the attending veterinarian, age, sex, and weight of the animal, and the ability of the substance/molecule, agonist or antagonist to elicit a desired response in the animal. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at dosages and for periods of time necessary, to achieve the desired therapeutic or prophylactic result.

In some embodiments, an anti-IL17A antibody, an IL17Ra ECD polypeptide, an IgG Fc variant polypeptide, or a polypeptide or pharmaceutical composition comprising such is administered parenterally, by subcutaneous administration, intravenous infusion, or intramuscular injection. In some embodiments, an anti-IL17A antibody, an IL17Ra ECD polypeptide, an IgG Fc variant polypeptide, or a polypeptide or pharmaceutical composition comprising such is administered as a bolus injection or by continuous infusion over a period of time. In some embodiments, an anti-IL17A antibody, an IL17Ra ECD polypeptide, an IgG Fc variant polypeptide, or a polypeptide or pharmaceutical composition comprising such is administered by an intramuscular, an intraperitoneal, an intracerebrospinal, a subcutaneous, an intra-arterial, an intrasynovial, an intrathecal, or an inhalation route.

Anti-IL17A antibodies, IL17Ra ECD polypeptides, IgG Fc variant polypeptides, or polypeptides or pharmaceutical compositions comprising such may be administered in an amount in the range of 0.1 mg/kg bodyweight to 100 mg/kg bodyweight per dose. In some embodiments, anti-IL17A antibodies, IL17Ra ECD polypeptides, IgG Fc variant polypeptides, or polypeptides or pharmaceutical compositions may be administered in an amount in the range of 0.5 mg/kg body weight to 50 mg/kg bodyweight per dose. In some embodiments, anti-IL17A antibodies, IL17Ra ECD polypeptides, IgG Fc variant polypeptides, or polypeptides or pharmaceutical compositions may be administered in an amount in the range of 1 mg/kg bodyweight to 10 mg/kg bodyweight per dose. In some embodiments, anti-IL17A antibodies, IL17Ra ECD polypeptides, IgG Fc variant polypeptides, or polypeptides or pharmaceutical compositions may be administered in an amount in the range of 0.5 mg/kg bodyweight to 100 mg/kg body, in the range of 1 mg/kg bodyweight to 100 mg/kg bodyweight, in the range of 5 mg/kg bodyweight to 100 mg/kg bodyweight, in the range of 10 mg/kg bodyweight to 100 mg/kg bodyweight, in the range of 20 mg/kg bodyweight to 100 mg/kg bodyweight, in the range of 50 mg/kg bodyweight to 100 mg/kg bodyweight, in the range of 1 mg/kg bodyweight to 10 mg/kg bodyweight, in the range of 5 mg/kg bodyweight to 10 mg/kg bodyweight, in the range of 0.5 mg/kg bodyweight to 10 mg/kg bodyweight, or in the range of 5 mg/kg body weight to 50 mg/kg body weight.

In some embodiments, an anti-IL17A antibody, an IL17Ra ECD polypeptide, an IgG Fc variant polypeptide, or a polypeptide or pharmaceutical composition comprising such can be administered to a companion animal at one time or over a series of treatments. For example, an anti-IL17A antibody, an IL17Ra ECD polypeptide, an IgG Fc variant polypeptide, or a polypeptide or pharmaceutical composition comprising such may be administered at least once, more than once, at least twice, at least three times, at least four times, or at least five times.

In some embodiments, the dose is administered once per week for at least two or three consecutive weeks, and in some embodiments, this cycle of treatment is repeated two or more times, optionally interspersed with one or more weeks of no treatment. In other embodiments, the therapeutically effective dose is administered once per day for two to five consecutive days, and in some embodiments, this cycle of treatment is repeated two or more times, optionally interspersed with one or more days or weeks of no treatment.

Administration “in combination with” one or more further therapeutic agents includes simultaneous (concurrent) and consecutive or sequential administration in any order. The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about a specified number of minutes. The term “sequentially” is used herein to refer to administration of two or more therapeutic agents where the administration of one or more agent(s) continues after discontinuing the administration of one or more other agent(s), or wherein administration of one or more agent(s) begins before the administration of one or more other agent(s). For example, administration of the two or more therapeutic agents are administered with a time separation of more than about a specified number of minutes. As used herein, “in conjunction with” refers to administration of one treatment modality in addition to another treatment modality. As such, “in conjunction with” refers to administration of one treatment modality before, during or after administration of the other treatment modality to the animal.

In some embodiments, the method comprises administering in combination with an anti-IL17A antibody, an IL17Ra ECD polypeptide, or a pharmaceutical composition comprising such, a NFκB inhibitor, a MAPKs inhibitor and a C/EBPs inhibitor. In some embodiments, the method comprises administering in combination with an anti-IL17A antibody, an IL17Ra ECD polypeptide, or a pharmaceutical composition comprising such, an anti-IL31 antibody, an anti-TNFα antibody, an anti-CD20 antibody, an anti-CD19 antibody, an anti-CD25 antibody, an anti-IL4 antibody, an anti-IL13 antibody, an anti-IL23 antibody, an anti-IgE antibody, an anti-CD11α antibody, anti-IL6R antibody, anti-α4-Intergrin antibody, an anti-IL12 antibody, an anti-IL1β antibody, or an anti-BlyS antibody.

Provided herein are methods of exposing to a cell an anti-IL17A antibody, an IL17Ra ECD polypeptide, or a pharmaceutical composition comprising such under conditions permissive for binding to IL17A. In some embodiments, the cell is exposed to the antibody, polypeptide, or pharmaceutical composition ex vivo. In some embodiments, the cell is exposed to the antibody, polypeptide, or pharmaceutical composition in vivo. In some embodiments, a cell is exposed to the anti-IL17A antibody, the IL17Ra ECD polypeptide, or the pharmaceutical composition under conditions permissive for binding to intracellular IL17A. In some embodiments, a cell is exposed to the anti-IL17A antibody, the IL17Ra ECD polypeptide, or the pharmaceutical composition under conditions permissive for binding to extracellular IL17A. In some embodiments, a cell may be exposed in vivo to the anti-IL17A antibody, the IL17Ra ECD polypeptide, or the pharmaceutical composition by any one or more of the administration methods described herein, including but not limited to, intraperitoneal, intramuscular, intravenous injection into the subject. In some embodiments, a cell may be exposed ex vivo to the anti-IL17A antibody, the IL17Ra ECD polypeptide, or the pharmaceutical composition by exposing the cell to a culture medium comprising the antibody, the polypeptide, or the pharmaceutical composition. In some embodiments, the permeability of the cell membrane may be affected by the use of any number of methods understood by those of skill in the art (such as electroporating the cells or exposing the cells to a solution containing calcium chloride) before exposing the cell to a culture medium comprising the antibody or the pharmaceutical composition.

In some embodiments, the binding results in a reduction of IL17A signaling function by the cell. In some embodiments, an anti-IL17A antibody or IL17Ra ECD polypeptide may reduce IL17A signaling function in a cell by at least 10%, at least 15%, at least 20%, at least 25%, at least 30%, at least 35%, at least 40%, at least 45%, at least 50%, at least 60%, at least 70%, at least 80%, at least 90%, or 100% compared to IL17A signaling function in the absence of the antibody or polypeptide, as measured by a reduction in IL6 secretion. In some embodiments, the reduction in IL17A signaling function or the reduction in IL6 secretion is between 10% and 15%, between 10% and 20%, between 10% and 25%, between 10% and 30%, between 10% and 35%, between 10% and 40%, between 10% and 45%, between 10% and 50%, between 10% and 60%, between 10% and 70%, between 10% and 80%, between 10% and 90%, between 10% and 100%, between 15% and 20%, between 15% and 25%, between 15% and 30%, between 15% and 35%, between 15% and 40%, between 15% and 45%, between 15% and 50%, between 15% and 60%, between 15% and 70%, between 15% and 80%, between 15% and 90%, between 15% and 100%, between 20% and 25%, between 20% and 30%, between 20% and 35%, between 20% and 40%, between 20% and 45%, between 20% and 50%, between 20% and 60%, between 20% and 70%, between 20% and 80%, between 20% and 90%, between 20% and 100%, between 25% and 30%, between 25% and 35%, between 25% and 40%, between 25% and 45%, between 25% and 50%, between 25% and 60%, between 25% and 70%, between 25% and 80%, between 25% and 90%, between 25% and 100%, between 30% and 35%, between 30% and 40%, between 30% and 45%, between 30% and 50%, between 30% and 60%, between 30% and 70%, between 30% and 80%, between 30% and 90%, between 30% and 100%, between 35% and 40%, between 35% and 45%, between 35% and 50%, between 35% and 60%, between 35% and 70%, between 35% and 80%, between 35% and 90%, between 35% and 100%, between 40% and 45%, between 40% and 50%, between 40% and 60%, between 40% and 70%, between 40% and 80%, between 40% and 90%, between 40% and 100%, between 45% and 50%, between 45% and 60%, between 45% and 70%, between 45% and 80%, between 45% and 90%, between 45% and 100%, between 50% and 60%, between 50% and 70%, between 50% and 80%, between 50% and 90%, between 50% and 100%, between 60% and 70%, between 60% and 80%, between 60% and 90%, between 60% and 100%, between 70% and 80%, between 70% and 90%, between 70% and 100%, between 80% and 90%, between 80% and 100%, or between 90% and 100%.

Provided herein are methods of using the anti-IL17A antibodies, polypeptides and polynucleotides for detection, diagnosis and monitoring of an IL17A-induced condition. Provided herein are methods of determining whether a companion animal will respond to anti-IL17A antibody therapy. In some embodiments, the method comprises detecting whether the animal has cells that express IL17A using an anti-IL17A antibody. In some embodiments, the method of detection comprises contacting the sample with an antibody, polypeptide, or polynucleotide and determining whether the level of binding differs from that of a reference or comparison sample (such as a control). In some embodiments, the method may be useful to determine whether the antibodies or polypeptides described herein are an appropriate treatment for the subject animal.

In some embodiments, the sample is a biological sample. The term “biological sample” means a quantity of a substance from a living thing or formerly living thing. In some embodiments, the biological sample is a cell or cell/tissue lysate. In some embodiments, the biological sample includes, but is not limited to, blood, (for example, whole blood), plasma, serum, urine, synovial fluid, and epithelial cells.

In some embodiments, the cells or cell/tissue lysate are contacted with an anti-IL17A antibody or IL17Ra ECD polypeptide and the binding between the antibody or the polypeptide and the cell is determined. When the test cells show binding activity as compared to a reference cell of the same tissue type, it may indicate that the subject would benefit from treatment with an anti-IL17A antibody or an IL17Ra ECD polypeptide. In some embodiments, the test cells are from tissue of a companion animal.

Various methods known in the art for detecting specific antibody-antigen binding can be used. Exemplary immunoassays which can be conducted include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. Appropriate labels include, without limitation, radionuclides (for example 125I, 131I, 35S, 3H, or 32P), enzymes (for example, alkaline phosphatase, horseradish peroxidase, luciferase, or β galactosidase), fluorescent moieties or proteins (for example, fluorescein, rhodamine, phycoerythrin, GFP, or BFP), or luminescent moieties (for example, Qdot™ nanoparticles supplied by the Quantum Dot Corporation, Palo Alto, Calif.). General techniques to be used in performing the various immunoassays noted above are known to those of ordinary skill in the art.

For purposes of diagnosis, the polypeptide including antibodies can be labeled with a detectable moiety including but not limited to radioisotopes, fluorescent labels, and various enzyme-substrate labels know in the art. Methods of conjugating labels to an antibody are known in the art. In some embodiments, the anti-IL17A antibodies need not be labeled, and the presence thereof can be detected using a second labeled antibody which binds to the first anti-IL17A antibody. In some embodiments, the anti-IL17A antibody can be employed in any known assay method, such as competitive binding assays, direct and indirect sandwich assays, and immunoprecipitation assays. Zola, Monoclonal Antibodies: A Manual of Techniques, pp. 147-158 (CRC Press, Inc. 1987). The anti-IL17A antibodies and polypeptides can also be used for in vivo diagnostic assays, such as in vivo imaging. Generally, the antibody or the polypeptide is labeled with a radionuclide (such as 111In, 99Tc, 14C, 131I, 125I, 3H, or any other radionuclide label, including those outlined herein) so that the cells or tissue of interest can be localized using immunoscintiography. The antibody may also be used as staining reagent in pathology using techniques well known in the art.

In some embodiments, a first antibody is used for a diagnostic and a second antibody is used as a therapeutic. In some embodiments, the first and second antibodies are different. In some embodiments, the first and second antibodies can both bind to the antigen at the same time, by binding to separate epitopes.

The following examples illustrate particular aspects of the disclosure and are not intended in anyway to limit the disclosure.

### EXAMPLES

### Example 1

Identification.of.Mouse.Monoclonal.Antibodies.that.Bind.to.Canine.IL7 [symbol] A

The nucleotide sequences encoding canine IL17A precursor protein with its native signal sequence (SEQ ID NO: 28) and either (1) a poly-His tag (canine IL17A-polyHis) or (2) human Fc (canine IL17A-huFc) on the C-terminal were synthesized and cloned into separate mammalian expression vectors. The resulting vectors were separately transfected into 293 cells.

The supernatant containing canine IL17A protein was collected and filtered. Canine IL17A-polyHis was affinity purified using Ni-NTA column (Catalog No. 17-5318-01, GE Healthcare Life Sciences) and canine IL17A-huFc was affinity purified using a protein A column (CAPTIVA Protein A Affinity Resin, Repligen).

A hybridoma bank generated from immunization of mice with human IL17A was screened for affinity to canine IL17A-polyHis (Catalog No. 70102-DNAE-50, Sino Biological) by enzyme linked immunosorbent assay (ELISA). Five hybridoma clones were identified for further characterization. Antibodies from clones A, B, C, D, and E were purified and their isotype determined using a Rapid Mouse Antibody Isotyping Kit (Catalog No. 26178, ThermoFisher Scientific). Clones A, C, D, and E were identified as mouse heavy chain isotype IgG1 and clone B was identified as mouse heavy chain isotype IgG2b. Each of the five clones were identified as having mouse kappa light chain.

### Example 2

Binding.Affinity.of.Monoclonal.Antibodies.to.Canine.IL7 [symbol] A.by.ELISA

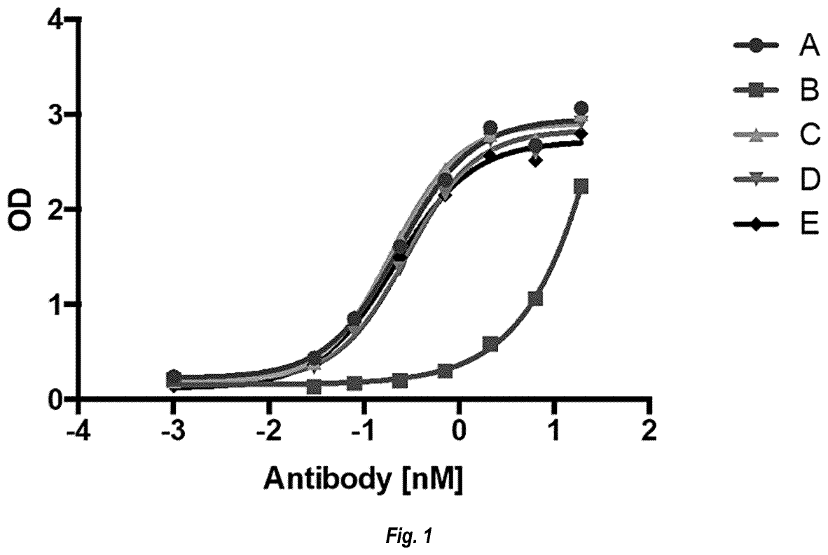

Binding affinity of monoclonal antibodies A, B, C, D, and E to canine IL17A-huFc polypeptide was analyzed by ELISA. In the binding ELISA performed, the wells were coated with anti-human Fc antibody. Canine IL17A-huFc protein was then added to the wells. Antibody purified from each of the five hybridoma clones was added to the wells at various concentrations (19.35 nM, 6.45 nM, 2.15 nM, 0.75 nM, 0.24 nM, 0.08 nM, 0.03 nM, and 0 nM). Goat anti-mouse Fc-HRP was added and color was developed. FIG. 1 shows the results of the binding ELISA. Table 3 below provides the EC50 values associated with the binding ELISA for each of the clones.

TABLE 3
Canine IL17A ELISA

| Antibody | EC50 (ng/mL) |
|---|---|
| A | 0.24 |
| B | 180 |
| C | 0.20 |
| D | 0.28 |
| E | 0.21 |

### Example 3

Affinity.of.Monoclonal.Antibodies.to.Canine.IL7 [symbol] A.by.Biosensor₌Based.Assay

Equilibrium dissociation constants (Kd) of the top four hybridoma candidates (clones A, C, D, and E) at a single concentration of 10 μg/mL were determined to each be less than 10 nM using biolayer interferometry. Briefly, antibody concentrations were measured by protein A assay using Biosensor OCTET (Forte Bio). Canine IL17A-huFc was captured to anti-human Fc bound biosensors. The association of anti-IL17A antibody from clones A, C, D, and E to the canine IL17A-huFc was monitored for 90 seconds. Dissociation was monitored for 600 seconds. A buffer only blank curve was subtracted to correct for any drift. The data were fit to a 1:1 binding model using FORTEBIO data analysis software to determine the kon, koff, and the Kd. The buffer for dilutions and all binding steps was: 20 mM phosphate, 150 mM NaCl, pH 7.2.

### Example 4

IL7 [symbol] Ra.Domains.for.Binding.IL7 [symbol] Ra.

Extracellular domains of canine IL17Ra responsible for binding to canine IL17A were identified, for example, SEQ ID NO: 33 and SEQ ID NO: 94. The nucleotide sequence encoding an extracellular domain of canine IL17Ra (canine IL17Ra ECD, SEQ ID NO: 33) with human Fc and a poly-His tag on the C terminal (canine IL17Ra ECD-huFc-polyHis, SEQ ID NO: 42) was synthesized and cloned into a mammalian expression vector. The resulting vector was transfected into 293 cells and CHOS cells. Canine IL17Ra ECD-huFc-polyHis was affinity purified using Protein A (CAPTIVA Protein A Affinity Resin, Repligen).

The binding affinity of canine IL17A-polyHis to canine IL17Ra ECD-huFc-polyHis was assessed using biolayer interferometry. Briefly, canine IL17A-polyHis was biotinylated and captured to streptavidin biosensors. The association of canine IL17Ra ECD-huFc-polyHis was monitored for 90 seconds. Dissociation was monitored for 600 seconds. A buffer only blank curve was subtracted to correct for any drift. The data were fit to a 1:1 binding model using FORTEBIO data analysis software to determine the kon, koff, and the Kd. The buffer for dilutions and all binding steps was: 20 mM phosphate, 150 mM NaCl, pH 7.2. Based on several runs of this assay, the Kd of canine IL17A-polyHis and canine IL17Ra ECD-huFc-polyHis was determined to be about 1.5×10-9 to about 4.2×10-9 M. These data suggest that the canine IL17Ra ECD fragment tested may have an affinity to IL17A sufficient for use as an IL17A antagonist, for example in the treatment of IL17A-induced conditions.

Extracellular domains of human IL17Ra, feline IL17Ra, and equine IL17Ra responsible for binding to IL17A were identified, for example, SEQ ID NO: 97, SEQ ID NO: 98, and SEQ ID NO: 99, respectively. The nucleotide sequence encoding an extracellular domain of human IL17Ra (human IL17Ra ECD, SEQ ID NO: 97) with human IgG4-Fc on the C terminal (human IL17Ra ECD-IgG4-Fc, SEQ ID NO: 40) may be prepared, cloned into a mammalian expression vector, and expressed in cells for isolation of the fusion protein. In addition, the nucleotide sequence encoding an extracellular domain of feline IL17Ra (feline IL17Ra ECD, SEQ ID NO: 98) with feline IgG-2 Fc on the C-terminal (feline IL17Ra ECD-feline IgG-2, SEQ ID NO: 41) may be prepared, cloned into a mammalian expression vector, and expressed in cells for the isolation of the fusion protein. Further, the nucleotide sequence encoding an extracellular domain of equine IL17Ra (equine IL17Ra ECD, SEQ ID NO: 99) with equine IgG-2 Fc on the C-terminal (equine IL17Ra ECD-equine IgG-2-Fc, SEQ ID NO: 42) may be prepared, cloned into a mammalian expression vector, and expressed in cells for the isolation of the fusion protein. The human, canine, feline, and equine IL17Ra ECD polypeptides and IL17Ra ECD/Fc fusion polypeptides described herein may be used in the treatment of IL17A-induced conditions, for example in humans, dogs, cats, or horses.

### Example 5

Anti₌IL7 [symbol] A.Monoclonal.Antibodies.Reduce.IL7 [symbol] A.Binding.to.IL7 [symbol] Ra.ECD

Each of the top four clones (A, C, D, and E) were determined to reduce the ability of canine IL17A to bind to canine IL17Ra ECD-huFc-polyHis in a biosensor-based assay. A complex between biotinylated canine IL17A-polyHis bound to streptavidin biosensors and anti-IL17A antibody (20 μg/mL) from one of each of clones A, B, C, D, and E was formed first. Then, the ability of canine IL17Ra ECD-huFc-PolyHis to bind to the IL17A/IL17A antibody complex was measured. The signal was compared to binding of IL17A to canine IL17Ra ECD-huFc-PolyHis in the absence of IL17A antibody. The biosensor signals observed from the IL17A/anti-IL17A antibody complex binding to the IL17Ra ECD were diminished with antibodies A, C, D, and E compared to the control (no antibody). These results suggest that Clone A, C, D, and E antibodies can reduce the ability of canine IL17A to bind to canine IL17Ra ECD.

### Example 6

Anti₌IL7 [symbol] A.Monoclonal.Antibodies.Reduce.IL7 [symbol] A.Signaling.Function

Whether clone A-E antibodies reduced IL17A signaling function was assessed using a H1080 cell-based functional assay. H1080 cells are a human fibrosarcoma cell line (ATCC-CCL121) that secretes pro-inflammatory cytokines IL6 and IL8 upon stimulation by either human or canine IL17A. Anti-IL17A antibodies may reduce the levels of secreted IL6 by blocking or reducing the ability of IL17A to bind IL17Ra on the surface of H1080 cells.

In this assay, the H1080 cells were incubated overnight with serial dilutions (10 nM, 3.3 nM, 1.1 nM, 0.33 nM, 0.11 nM, 0.03 nM, 0.01 nM, 0 nM) of purified anti-IL17A antibodies from clones A-E mixed with 1 nM canine IL17A. The amount of IL6 secreted from the cells into the medium was measured by a Human IL6 DUOSET ELISA kit (Catalog No. DY206-05, R&D Systems). A reduced level of IL6 secreted from H1080 cells was observed after treatment with each of the five anti-IL17A antibody clones compared to untreated cells. These results suggest that each of antibody clones A-E can reduce binding of IL17A to IL17Ra on the surface of H1080 cells and inhibit IL6 production. The antibody concentration at which a half-maximal response was observed (EC50) is summarized in Table 4, below. Clone A, C, D, and E antibodies appear to be more potent than clone B antibodies in this cell-based functional assay.

TABLE 4
H1080 Cell Functional Assay

| Antibody | EC50 (ng/mL) |
|---|---|
| A | 13 |
| B | 800 |
| C | 7 |
| D | 47 |
| E | 40 |

### Example 7

### Epitope.Binding.Immunoassay.

The IL17A epitope binding profiles of antibodies produced by clones A, B, C, D, and E were analyzed by competitive immunoassay. In this experiment, biotinylated canine IL17A-polyHis was immobilized on streptavidin sensor tips. The IL17A-bound sensor tips were exposed to Antibody 1. After a short wash, the sensor tips were then exposed to Antibody 2. If Antibody 2 failed to bind to the IL17A/Antibody 1 complex, the binding signal would not increase between exposure to Antibody 1 and exposure to Antibody 2. This would suggest that the two antibodies bound to the same or a closely related epitope and should be binned into the same epitope group. If Antibody 2 bound to the IL17A/Antibody 1 complex, then the binding signal would increase between exposure to Antibody 1 and exposure to Antibody 2. This scenario would suggest that the two antibodies belong to different epitope groups. In addition to controls, the following combinations of antibodies were used in the IL17A binding experiments: 1) Clone A followed by Clone A, B, C, D, and E; 2) Clone B followed by Clone A, B, C, D, and E; 3) Clone C followed by Clone A, B, C, D, and E; and 4) Clone D followed by Clone A, B, C, D, and E; and 5) Clone E followed by Clone A, B, C, D, and E.

Two different epitope binding groups were identified. Clones A, C, and E were identified as belonging to one group and Clone D was identified as belonging to a second group. Due to the weak affinity of clone B to IL17A, the epitope binding group for clone B was inconclusive.

### Example 8

Identification.of.Anti₌IL7 [symbol] A.Monoclonal.Antibody.Sequences

Hybridoma clones A, C, D, and E were pelleted and total RNA samples were extracted. Oligonucleotide primers for amplifying mouse immunoglobulin (Ig) variable domains were used to obtain cDNA using standard techniques. Variable regions of both heavy chains and light chains of the clones were amplified using in-house designed reverse primers. Amplified variable cDNAs were cloned into pRACE vector and plasmid DNA samples were prepared. The variable light chain (VL) and variable heavy chain (VH) of each of the four clones were sequenced and analyzed by sequence alignment (FIGS. 2A and B, respectively).

Clone A has a variable heavy chain sequence of SEQ ID NO: 35 and a variable light chain sequence of SEQ ID NO: 34; Clone C has a variable heavy chain sequence of SEQ ID NO: 25 and a variable light chain sequence of SEQ ID NO: 24; Clone D has a variable heavy chain sequence of SEQ ID NO: 37 and a variable light chain sequence of SEQ ID NO: 36; and Clone E has a variable heavy chain sequence of SEQ ID NO: 39 and a variable light chain sequence of SEQ ID NO: 38.

The CDRs of Clones A-E antibodies were determined using a combination of the Chothia, the Kabat, the AbM, and the contact numbering schemes or definitions.

Clone C has a CDR-H1 sequence of SEQ ID NO: 1, a CDR H2 sequence of SEQ ID NO: 2, a CDR-H3 sequence of SEQ ID NO: 3, a CDR-L1 sequence of SEQ ID NO: 8, a CDR-L2 sequence of SEQ ID NO: 9, and a CDR-L3 sequence of SEQ ID NO: 10.

Clone A has a CDR-H1 sequence of SEQ ID NO: 52, a CDR H2 sequence of SEQ ID NO: 53 or SEQ ID NO: 109, a CDR-H3 sequence of SEQ ID SEQ ID NO: 54, a CDR-L1 sequence of SEQ ID NO: 59 or SEQ ID NO: 111, a CDR-L2 sequence of SEQ ID NO: 60 or SEQ ID NO: 112, and a CDR-L3 sequence of SEQ ID NO: 61.

Clone E has a CDR-H1 sequence of SEQ ID NO: 66, a CDR H2 sequence of SEQ ID NO: 67 or SEQ ID NO: 114, a CDR-H3 sequence of SEQ ID NO: 68, a CDR-L1 sequence of SEQ ID NO: 73 or SEQ ID NO: 116, a CDR-L2 sequence of SEQ ID NO: 74 or SEQ ID NO: 117; and a CDR-L3 sequence of SEQ ID NO: 75.

Clone D has a CDR-H1 sequence of SEQ ID NO: 80, a CDR H2 sequence of SEQ ID NO: 81 or SEQ ID NO: 119, a CDR-H3 sequence of SEQ ID NO: 82, a CDR-L1 sequence of SEQ ID NO: 87 or SEQ ID NO: 121, a CDR-L2 sequence of SEQ ID NO: 88 or SEQ ID NO: 122, and a CDR-L3 sequence of SEQ ID NO: 89.

### Example 9

Expression.and.Purification.of.Murine₌Canine.Chimeric.and.Caninized.IL7 [symbol] .Clone.C.Antibodies.from.CHO.Cells

Nucleotide sequences encoding a chimeric antibody were designed for a fusion of murine Clone C VH (SEQ ID NO: 25) and VL (SEQ ID NO: 24) to canine constant heavy chain and canine constant light chain. The nucleotide sequences were chemically synthesized and inserted into an expression vector suitable for transfection into a CHO host cell. After transfection into CHO cells, the light chain or heavy chain protein or both were secreted from the cell and purified by column chromatography. For example, chimeric Clone C having canine IgG-B (SEQ ID NO: 27) and canine kappa constant chain (SEQ ID NO: 26) was purified by single step Protein A column chromatography.

Murine Clone C VH and VL were caninized by searching and selecting proper canine germline antibody sequences as a template for CDR grafting, followed by protein modeling (SEQ ID NO: 15 and SEQ ID NO: 16). Caninized Clone C comprising caninized Clone C VH and canine IgG-B (SEQ ID NO: 18) and caninized Clone C VL and canine kappa constant region (SEQ ID NO: 21) was expressed and purified in a single step with a protein A column (Catalog No: 17127901, GE Healthcare Life Sciences). The antibody expression vectors were then used to perform pilot-scale transfection in CHO S cells using the FreestyleMax™ transfection reagent (Life Technologies). The supernatant was harvested by clarifying the conditioned media. Protein was purified with a single pass Protein A chromatography step and used for further investigation.

Other chromatographic methods that may be used for purification include, ion exchange column chromatography, hydrophobic interaction column chromatography, mixed mode column chromatography such as CHT, or multimodal mode column chromatography such as CAPTOMMC (Catalog No. 17371605, GE Healthcare Life Sciences). Low pH or other viral inactivation and viral removal steps may also be applied. The purified protein may be admixed with excipients, and sterilized by filtration to prepare a pharmaceutical composition. The pharmaceutical composition comprising the IL17A antibodies described herein may be administered to a dog with an IL17-induced condition, such as atopic dermatitis in an amount sufficient to bind IL17A.

### Example 10

Demonstration.of.IL7 [symbol] A.Binding.Activity

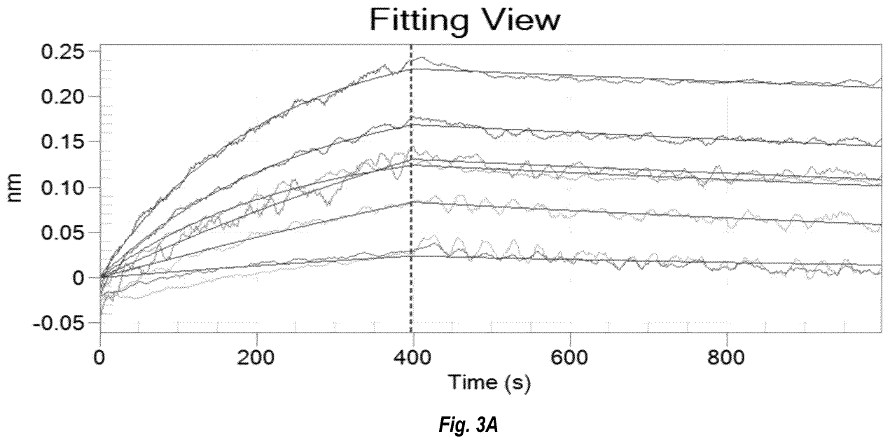

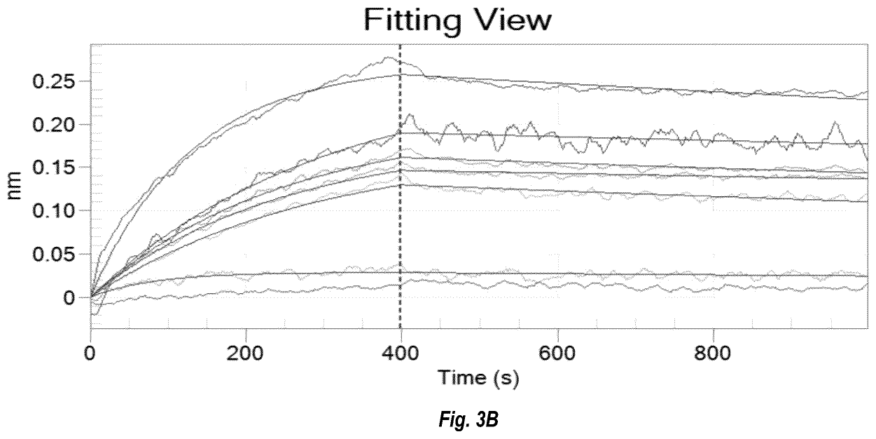

Hybridoma Clone C having VL SEQ ID NO: 24 and VH SEQ ID NO: 25 exhibited affinity to canine IL17A with kinetics potentially sufficient for therapeutic activity. The affinity to canine IL17A was preserved in caninized Clone C (FIG. 3) and chimeric Clone C (data not shown). The caninized Clone C and chimeric Clone C antibodies were prepared as described in Example 9.

The binding analysis was performed using a Biosensor OCTET as follows. Briefly, canine IL17A-polyHis, which was expressed and purified from CHO-S cells, was biotinylated using EZ-LINK NHS-LC-biotin (Catalog No. 21336, Thermo Scientific). Free, unreacted biotin was removed from biotinylated IL17A by dialysis. Biotinylated canine IL17A was captured on streptavidin sensor tips (Catalog No. 18-509, ForteBio). The association of different concentrations (0 nM, 18.33 nM, 45.87 nM, 110 nM, 220 nM) of hybridoma Clone C or caninized Clone C antibody to canine IL17A was monitored for 90 seconds. Dissociation was monitored for 600 seconds. A buffer only blank curve was subtracted to correct for any drift. The data were fit to a 1:1 binding model using FORTEBIO data analysis software to determine the kon, koff, and the Kd. The buffer for dilutions and all binding steps was: 20 mM phosphate, 150 mM NaCl, pH 7.2.

The Kd of hybridoma Clone C and canine IL17A-polyHis was 7.9×10-9 M (FIG. 3A) and the Kd of caninized Clone C and canine IL17A-polyHis was 1.4×10-9 M (FIG. 3B).

### Example 11

Demonstration.of.IL7 [symbol] F.Binding.Activity

In addition to IL17A, there are other members of the IL17 family, including IL17F. The binding affinity of IL17F homodimer to caninized Clone C (prepared as described in Example 9) was assessed using biolayer interferometry. It was determined that caninized Clone C also binds to canine IL17F-polyHis with a Kd of 4.1×10-9 M.

The nucleotide sequence encoding IL17F precursor polypeptide (SEQ ID NO: 31) with a poly-His tag (canine IL17F-polyHis) on the C-terminal was synthesized and cloned into a mammalian expression vector. The resulting vector was transfected into 293 cells. The supernatant was collected and filtered, and canine IL17F-polyHis protein was affinity purified using a Ni-NTA column (CAPTIVA Protein A Affinity Resin, Repligen).

The binding analysis was performed using a biosensor OCTET as follows. Briefly, canine IL17F-polyHis was biotinylated using EZ-LINK NHS-LC-biotin (Catalog No. 21336, Thermo Scientific). Free, unreacted biotin was removed from biotinylated IL17F-polyHis by dialysis. Biotinylated canine IL17F was captured on streptavidin sensor tips (Catalog No. 18-509, ForteBio). The association of different concentrations (0 nM, 2 nM, 10.1 nM, 21.3 nM, 43.3 nM, 86.7 nM, 124 nM) of the caninized Clone C antibody and canine IL17F-polyHis was monitored for ninety seconds. Dissociation was monitored for 600 seconds. A buffer only blank curve was subtracted to correct for any drift. The data were fit to a 1:1 binding model using FORTEBIO data analysis software to determine the kon, koff, and the Kd. The buffer for dilutions and all binding steps was: 20 mM phosphate, 150 mM NaCl, pH 7.2.

### Example 12

Demonstration.that.Caninized.Clone.C.Inhibits.IL7 [symbol] .Signaling

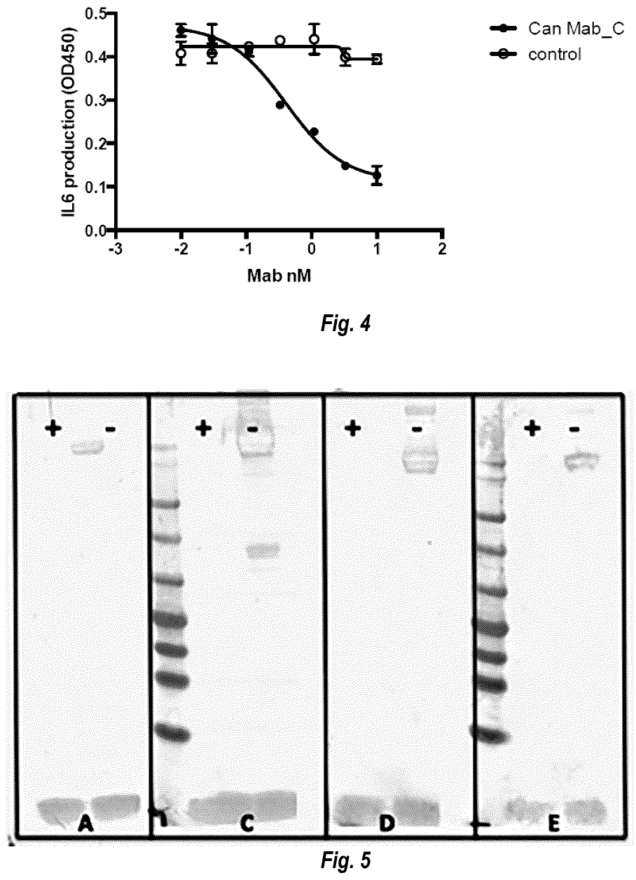

The ability of caninized Clone C antibody (prepared as described in Example 9) to affect human IL17A signaling function was assessed using the H1080 cell-based functional assay described in Example 6. H1080 cells were incubated overnight with serial dilutions of purified caninized Clone C antibody mixed with 1 nM canine IL17A. The amount of IL6 secreted from the cells into the medium was measured by a Human IL6 DUOSET ELISA kit (Catalog No. DY206-05, R&D Systems). The levels of IL6 produced by H1080 cells treated with caninized Clone C antibody or an unrelated caninized IgG B antibody as a negative control are shown in FIG. 4. In this assay, the IC50 of the caninized Clone C antibody was 0.41×10-9 M, suggesting that caninized Clone C antibody inhibits the IL17A signaling pathway.

### Example 13

Clones.A?.C?.D?.and.E.Cross.React.to.Feline.and.Equine.IL7 [symbol] A.

The nucleotide sequences encoding feline IL17A precursor protein with its native signal sequence (SEQ ID NO: 30) and a poly-His tag on the C-terminal (feline IL17A-polyHis) and equine IL17A precursor protein with its native signal sequence (SEQ ID NO: 29) and a poly-His tag on the C-terminal (equine IL17A-polyHis) were synthesized and cloned into separate mammalian expression vectors. The resulting vectors were separately transfected into 293 cells. The supernatant containing feline IL17A-polyHis or equine IL17A-polyHis was collected and filtered, and the IL17A proteins affinity purified using a Ni-NTA column.

The binding analysis was performed using a Biosensor OCTET as follows. Both feline IL17A-polyHis and equine IL17A-polyHis were biotinylated and immobilized to streptavidin biosensors. The association of monoclonal antibody Clones A, C, D, and E (20 μg/mL) to either feline or canine IL17A-polyHis was monitored for 90 seconds. Dissociation was monitored for 600 seconds. A buffer only blank curve was subtracted to correct for any drift. The data were fit to a 1:1 binding model using FORTEBIO data analysis software to determine the kon, koff, and the Kd. The buffer for dilutions and all binding steps was: 20 mM phosphate, 150 mM NaCl, pH 7.2. The biolayer interferometry analysis indicated that monoclonal antibody Clones A, C, D, and E have binding affinity to feline and equine IL17A. The Kd of feline IL17A to monoclonal antibody Clone D and Clone E was 2.4×10-9 M and 5.7×10-9 M, respectively. Weak binding signals of Clone A to feline IL17A and Clone C to feline IL17A were observed. The Kd of equine IL17A to Clone A was 5.3×10 9 M, to Clone C was 1.3×10-10 M, to Clone D was 2.1×10-10 M, and to Clone E was 1.8×10-10 M.

### Example 14

Affinity.of.IL7 [symbol] A.Monoclonal.Antibodies.to.IL7 [symbol] A.Proteins.by.Western.Analysis

Canine IL17A-hFc was separated by SDS-PAGE in the presence of Dithiothreitol (DTT) (reducing conditions) or the absence of DTT (non-reducing conditions). The protein was transferred to PVDF membrane and probed using Clone A, C, D, or E antibody followed by goat anti-mouse IgG-HRP. Immunoreactive positive signals were only observed in non-reducing samples, suggesting that the epitope for each of Clone A, C, D, and E antibodies may be discontinuous or conformational (FIG. 5).

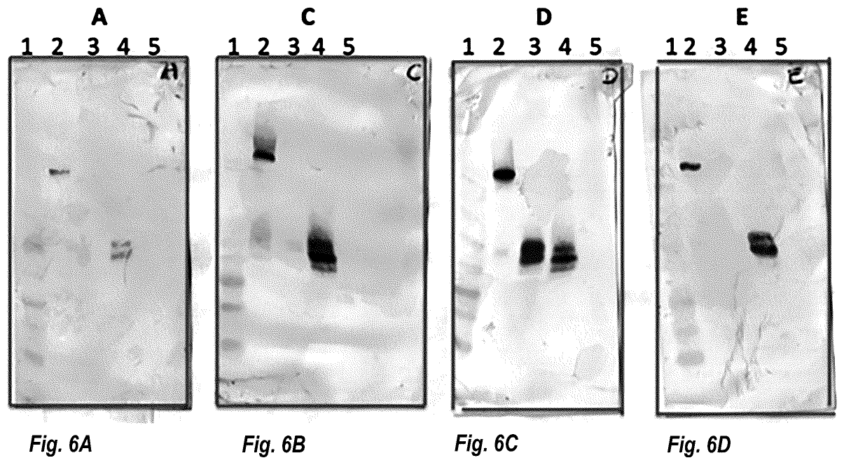

Canine IL17A-hFc, feline IL17A-polyHis, equine IL17A-polyHis, and canine IL17F-polyHis (0.6 μg) were each separated by SDS-PAGE and the proteins transferred to PVDF membranes. The blots were probed using Clone A, C, D, or E antibody followed by goat anti-mouse IgG-HRP. The Western blots are shown in FIG. 6 and the presence or absence of an immunoreactivity signal is summarized in Table 5, below. In this Western analysis, Clone A, C, and E antibodies immunoreacted to canine and equine IL17A, but not to feline IL17A or canine IL17F. Clone D antibody immunoreacted to canine, feline, and equine IL17A, but not to canine IL17F.

TABLE 5
Immunoreactivity to IL17A protein target by Western analysis

Protein Target

| Monoclonal | Canine | Feline | Equine | Canine |
|---|---|---|---|---|
| Antibody | IL17A | IL17A | IL17A | IL17F |
| Clone A | Yes | No | Yes | No |
| Clone C | Yes | No | Yes | No |
| Clone D | Yes | Yes | Yes | No |
| Clone E | Yes | No | Yes | No |

### Example 15

Identification.of.IL7 [symbol] A.Binding.Epitope.for.Clone.C.Antibody.

To identify the canine IL17A epitope that is recognized by Clone C antibody, the antibody's affinity to a series of mutant canine IL17A-hFc-polyHis proteins was considered by Western analysis. The results described in Example 13 showed that Clone C antibody immunoreacted to canine and equine IL17A, but not to feline IL17A. To identify potential IL17A epitope binding sites for Clone C, the amino acid sequences and 3-D protein structure models of canine, equine, and feline IL17A proteins were compared. Three segments of amino acid sequence that are generally conserved between canine and equine IL17A proteins and that are generally divergent when compared to the feline IL17A sequence were identified as potential epitope binding regions for Clone C antibody.

Three mutant canine IL17A-hFc-polyHis polypeptides were constructed with each having amino acid substitution(s) in one of the three segments identified by sequence and 3-D structure comparison (Mutants 1, 2, 3). The amino acid substitutions of Mutants 1-3 were derived from the amino acids divergent between the canine and feline IL17A sequences. (See Table 6, below). A fourth mutant canine IL17A-hFc-polyHis polypeptide (Mutant 4) was constructed that harbored all the amino acid substitutions of Mutants 1, 2, and 3. A fifth mutant canine IL17A-hFc-polyHis polypeptide was constructed having a C-terminal deletion of amino acids 126-131 of SEQ ID NO: 22.

TABLE 6

| Canine IL17A-hFc | Amino acid modification(s) | +DDT | −DDT |
|---|---|---|---|
| mutant | (based on SEQ ID NO: 22) | lane | lane |
| Mutant 1 | R71L, L73W, I82T | 3 | 4 |
| Mutant 2 | R32A, T34K, N35S, N44R | 5 | 6 |
| Mutant 3 | Q103R | 7 | 8 |
| Mutant 4 | R32A, T34K, N35S, N44R, | 9 | 10 |
| | R71L, L73W, I82T, Q103R | | |
| Mutant 5 | C-terminal deletion of | 1 | 2 |
| | amino acids 126-131 | | |

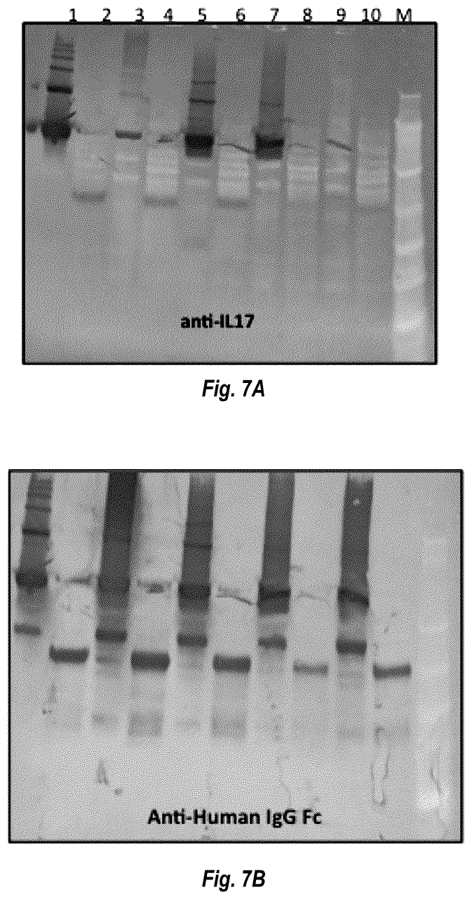

Plasmid constructs containing nucleotide sequences encoding each of the canine IL17A-hFC-polyHis mutants were transiently transfected into 293 cells and the supernatants concentrated 3 fold. Each mutant was separated by SDS-PAGE (25 μL per lane) in the presence or absence of DTT and the proteins transferred to a PVDF membrane. The blot was probed using either Clone C antibody or anti-human IgG Fc (control). The Western blots are shown in FIG. 7. The signal in lane 3 of the Clone C antibody blot is reduced compared to the control blot suggesting that Clone C antibody binds to canine IL17A in the region having the Mutant 1 mutations (R71L, L73W, 182T). The Western analysis of mutant canine IL17A suggests that Clone C antibody binds to an epitope within amino acids 65 to 88 of SEQ ID NO: 22, for example an epitope comprising the amino acid sequence of SEQ ID NO: 23 or SEQ ID NO: 51.

### Example 16

### Modification.of.Canine.Fc.Complement.Binding.Activity.

Canine IgG-B Fc (SEQ ID NO: 45) and canine IgG-C Fc (SEQ ID NO: 46) have complement activity. To potentially reduce the binding of C1 q to IgG-B Fc and IgG-C Fc, and/or potentially reduce complement-mediated immune responses, IgG-B Fc and IgG C Fc variants may be prepared having an amino acid substitution of Lys at amino acid position 110 of SEQ ID NO:45 or of Lys at amino acid position 108 of SEQ ID NO: 46 with any amino acid except Lys. These amino acid substitutions were identified after analysis of the protein sequence and 3-D structure modeling of canine IgG-B and IgG C compared to canine IgG-A and IgG-D, which are understood to not exhibit complement activity.

For example, canine IgG-B Fc variant 1 may be prepared by substituting the Lys at amino acid position 110 of SEQ ID NO: 45 with Arg (SEQ ID NO: 47) and canine IgG-C Fc variant 1 may be prepared by substituting the Lys at position 108 of SEQ ID NO: 46 with Arg (SEQ ID NO: 48).

The determine the binding affinity of C1q to canine IgG-B Fc variant 1, a biosensor binding analysis was performed. In this assay, the affinity of C1q to a fusion protein of a canine IL4 receptor ECD and IL13 receptor ECD (IL4R/IL13R) and canine IgG-B Fc wild-type (IL4R/IL13R-canine IgG-B, SEQ ID NO: 95) or to canine IgG-B Fc variant 1 (IL4R/IL13R-canine IgG-B variant 1, SEQ ID NO: 96) was tested. Briefly, canine IL4 was biotinylated. Biotinylated canine IL4 was captured on streptavidin sensor tips. Either IL4R/IL13R-canine IgG-B wild-type (25 ug/mL) or IL4R/IL13R-canine IgG-B variant 1 (25 μg/mL) were complexed to the IL4-bound biosensors. Subsequently, the complex was used to bind human C1 q at 250 μg/mL (Catalog No. 204876-1 MG; Sigma Aldrich). Then, the ability of human C1q to bind to either complex was measured. Reduced binding between human C1q and IL4R/IL13R-canine IgG-B variant 1 was observed when compared to IL4R/IL13R-canine IgG-B wild-type.

### Example 17

Modification.of.Canine.Fc.CD7 [symbol] .Binding.Activity.

Canine IgG-B Fc (SEQ ID NO: 45) and canine IgG-C Fc (SEQ ID NO: 46) have CD16 binding activity. To potentially reduce the binding of CD16 to IgG-B Fc and IgG-C Fc, and/or potentially reduce antibody-dependent cell-mediated cytotoxicity (ADCC), canine IgG-B Fc and IgG C Fc variants may be prepared having one or more of the amino acid substitutions listed in Table 7. The amino acid substitution(s) were identified after analysis of the protein sequence and 3-D structure modeling of canine IgG-B and IgG C compared to canine IgG-A and IgG-D, which are understood to not exhibit ADCC activity.

TABLE 7

Original residue position

| Canine IgG-B Fc | Canine IgG-C Fc | |
|---|---|---|
| (SEQ ID NO: 45) | (SEQ ID NO: 46) | Substitution(s) |
| Glu (55) | Glu (53) | Any amino acid |
| | | except Glu |
| Lys (114) | Lys (112) | Any amino acid |
| | | except Lys |
| Ala (115) | Ala (113) | Any amino acid |
| | | except Ala |

For example, canine IgG-B-Fc variant 2 (SEQ ID NO: 49) may be prepared by substituting Glu at position 55 of canine IgG-B Fc (SEQ ID NO: 45) with Gly, Lys at position 114 with Ile, and Ala at position 115 with Gly. As another example, canine IgG-C-Fc variant 2 (SEQ ID NO: 50) may be prepared by substituting Glu at position 53 of canine IgG-C Fc (SEQ ID NO: 46) with Gly, Lys at position 112 with Ile, and Ala at position 113 with Gly.

The binding of any of the canine IgG-B Fc or IgG-C Fc variants to CD16 may be determined and compared to the binding of another IgG Fc to CD16 (e.g., the corresponding canine IgG-B Fc or IgG-C Fc wild-type, other canine IgG Fc variant, etc.). The binding assay described in Example 16 may be used.

1. A monoclonal antibody that binds to canine IL17A, wherein the antibody comprises:
a) (i) a CDR-H1 sequence comprising the amino acid sequence of SEQ ID NO: 52, a CDR-H2 sequence comprising the amino acid sequence of SEQ ID NO: 53 or SEQ ID NO: 109, and a CDR-H3 sequence comprising the amino acid sequence of SEQ ID NO: 54, and
(ii) a CDR-L1 sequence comprising the amino acid sequence of SEQ ID NO: 59 or SEQ ID NO: 111, a CDR-L2 sequence comprising the amino acid sequence of SEQ ID NO: 60 or SEQ ID NO: 112, and a CDR-L3 sequence comprising the amino acid sequence of SEQ ID NO: 61; or
b) (i) a CDR-H1 sequence comprising the amino acid sequence of SEQ ID NO: 80, a CDR-H2 sequence comprising the amino acid sequence of SEQ ID NO: 81 or SEQ ID NO: 119, and a CDR-H3 sequence comprising the amino acid sequence of SEQ ID NO: 82, and
(ii) a CDR-L1 sequence comprising the amino acid sequence of the amino acid sequence of SEQ ID NO: 87 or SEQ ID NO: 121, a CDR-L2 sequence comprising the amino acid sequence of SEQ ID NO: 88 or SEQ ID NO: 122; and a CDR-L3 sequence comprising the amino acid sequence of SEQ ID NO: 89; or
c) (i) a CDR-H1 sequence comprising the amino acid sequence of SEQ ID NO: 66, a CDR-H2 sequence comprising the amino acid sequence of SEQ ID NO: 67 or SEQ ID NO: 114, and a CDR-H3 sequence comprising the amino acid sequence of SEQ ID NO: 68, and
(ii) a CDR-L1 sequence comprising the amino acid sequence of SEQ ID NO: 73 or SEQ ID NO: 116, a CDR-L2 sequence comprising the amino acid sequence of SEQ ID NO: 74 or SEQ ID NO: 117; and a CDR-L3 sequence comprising the amino acid sequence of SEQ ID NO: 75.

2. The monoclonal antibody of claim 1, wherein the antibody binds to canine IL17A with a dissociation constant (K_{d}) of less than 1×10⁻⁸ M, as measured by biolayer interferometry.

3. The monoclonal antibody of claim 1, wherein the antibody is a caninized, a felinized, an equinized, or a chimeric antibody.

4. The monoclonal antibody of claim 1, wherein the antibody comprises:
a. (i) a variable light chain sequence having at least 95% identity to the amino acid sequence of SEQ ID NO: 34; (ii) a variable heavy chain sequence having at least 95% sequence identity to the amino acid sequence of SEQ ID NO: 35; or (iii) a variable light chain sequence as in (i) and a variable heavy chain sequence as in (ii); or
b. (i) a variable light chain sequence having at least 95% sequence identity to the amino acid sequence of SEQ ID NO: 36; (ii) a variable heavy chain sequence having at least 95% sequence identity to the amino acid sequence of SEQ ID NO: 37; or (iii) a variable light chain sequence as in (i) and a variable heavy chain sequence as in (ii); or
c. (i) a variable light chain sequence having at least 95% sequence identity to the amino acid sequence of SEQ ID NO: 38; (ii) a variable heavy chain sequence having at least 95% sequence identity to the amino acid sequence of SEQ ID NO: 39; or (iii) a variable light chain sequence as in (i) and a variable heavy chain sequence as in (ii).

5. The monoclonal antibody of claim 1, wherein the antibody comprises (a) a canine heavy chain constant region selected from an IgG-A, IgG-B, IgG-C, and IgG-D constant region; (b) a feline heavy chain constant region selected from an IgG-1a, IgG-1 b, and IgG-2 constant region; or (c) an equine heavy chain constant region selected from an IgG-1, IgG-2, IgG-3, IgG-4, IgG-5, IgG-6, and IgG-7 constant region.

6. The monoclonal antibody of claim 1, wherein the antibody is an antibody fragment chosen from an Fv, scFv, Fab, Fab′, F(ab′)2, and Fab′-SH fragment.

7. A nucleic acid encoding the monoclonal antibody of claim 1.

8. A host cell comprising the nucleic acid of claim 7.

9. A method of producing a monoclonal antibody comprising culturing the host cell of claim 8 and isolating the monoclonal antibody.

10. A pharmaceutical composition comprising the monoclonal antibody of claim 1 and a pharmaceutically acceptable carrier.

11. A method of treating a companion animal species having an IL17A-induced condition, the method comprising administering to the companion animal species a therapeutically effective amount of the monoclonal antibody of claim 1.

12. The method of claim 11, wherein the companion animal species is canine, feline, or equine.

13. A method of reducing IL17A signaling function in a cell, the method comprising exposing to the cell the monoclonal antibody of claim 1 under conditions permissive for binding of the antibody to extracellular IL17A, thereby reducing binding to IL17A receptor and/or reducing IL17A signaling function by the cell.

14. A method for detecting IL17A in a sample from a companion animal species comprising contacting the sample with the monoclonal antibody of claim 1 under conditions permissive for binding of the antibody to IL17A, and detecting whether a complex is formed between the monoclonal antibody and IL17A in the sample.

15. The monoclonal antibody of claim 1, further comprising one or more of:
a. one or more of (i) a variable region heavy chain framework 1 (HC-FR1) sequence of SEQ ID NO: 55; (ii) a HC-FR2 sequence of SEQ ID NO: 56 or SEQ ID NO: 110; (iii) a HC-FR3 sequence of SEQ ID NO: 57; (iv) a HC-FR4 sequence of SEQ ID NO: 58; (v) a variable region light chain framework 1 (LC-FR1) sequence of SEQ ID NO: 62; (vi) an LC-FR2 sequence of SEQ ID NO: 63 or SEQ ID NO: 113; (vii) an LC-FR3 sequence of SEQ ID NO: 64; and/or (vii) an LC-FR4 sequence of SEQ ID NO: 65; or
b. one or more of (i) a variable region heavy chain framework 1 (HC-FR1) sequence of SEQ ID NO: 83; (ii) a HC-FR2 sequence of SEQ ID NO: 84 or SEQ ID NO: 120; (iii) a HC-FR3 sequence of SEQ ID NO: 85; (iv) a HC-FR4 sequence of SEQ ID NO: 86; (v) a variable region light chain framework 1 (LC-FR1) sequence of SEQ ID NO: 90; (vi) an LC-FR2 sequence of SEQ ID NO: 91 or SEQ ID NO: 123; (vii) an LC-FR3 sequence of SEQ ID NO: 92; and/or (vii) an LC-FR4 sequence of SEQ ID NO: 93; or
c. one or more of (i) a variable region heavy chain framework 1 (HC-FR1) sequence of SEQ ID NO: 69; (ii) a HC-FR2 sequence of SEQ ID NO: 70 or SEQ ID NO: 115; (iii) a HC-FR3 sequence of SEQ ID NO: 71; (iv) a HC-FR4 sequence of SEQ ID NO: 72; (v) a variable region light chain framework 1 (LC-FR1) sequence of SEQ ID NO: 76; (vi) an LC-FR2 sequence of SEQ ID NO: 77 or SEQ ID NO: 118; (vii) an LC-FR3 sequence of SEQ ID NO: 78; and/or (vii) an LC-FR4 sequence of SEQ ID NO: 79.

16. The monoclonal antibody of claim 1, wherein the monoclonal antibody binds to an epitope within amino acids 65 to 88 of SEQ ID NO: 22.

17. The monoclonal antibody of claim 16, wherein the monoclonal antibody binds to SEQ ID NO: 23 or SEQ ID NO: 51.

18. The antibody of claim 4, wherein the antibody comprises:
a. a variable light chain sequence of SEQ ID NO: 34 and a variable heavy chain sequence of SEQ ID NO: 35; or
b. a variable light chain sequence of SEQ ID NO: 36 and a variable heavy chain sequence of SEQ ID NO: 37; or
c. a variable light chain sequence of SEQ ID NO: 38 and a variable heavy chain sequence of SEQ ID NO: 38.

19. A monoclonal antibody that binds to canine IL17A, wherein the antibody comprises:
(i) a CDR-H1 comprising the amino acid sequence of SEQ ID NO: 52, a CDR H2 comprising the amino acid sequence of SEQ ID NO: 53, and a CDR-H3 comprising the amino acid sequence of SEQ ID NO: 54, and
(ii) a CDR-L1 sequence comprising the amino acid sequence of SEQ ID NO: 59 or SEQ ID NO: 111, a CDR-L2 sequence comprising the amino acid sequence of SEQ ID NO: 60 or SEQ ID NO: 112, and a CDR-L3 sequence comprising the amino acid sequence of SEQ ID NO: 61.

20. A monoclonal antibody that binds to canine IL17A, wherein the antibody comprises:
(i) a CDR-H1 comprising the amino acid sequence of SEQ ID NO: 66, a CDR H2 comprising the amino acid sequence of SEQ ID NO: 67 or SEQ ID NO: 114, and a CDR-H3 comprising the amino acid sequence of SEQ ID NO: 68, and
(ii) a CDR-L1 sequence comprising the amino acid sequence of SEQ ID NO: 73 or SEQ ID NO: 116, a CDR-L2 sequence comprising the amino acid sequence of SEQ ID NO: 74 or SEQ ID NO: 117, and a CDR-L3 sequence comprising the amino acid sequence of SEQ ID NO: 75.


SEQUENCE LISTING (123 sequences)

SEQ ID NO: 1 - protein, 10 aa, synthetic construct
  REGION 1..10: note = Variable heavy chain CDR-H1 amino acid sequence of mouse antibody clone C
GFTFSSYGMS

SEQ ID NO: 2 - protein, 17 aa, synthetic construct
  REGION 1..17: note = Variable heavy chain CDR-H2 amino acid sequence of mouse antibody clone C
IINSNGGSTYYPDSVKG

SEQ ID NO: 3 - protein, 11 aa, synthetic construct
  REGION 1..11: note = Variable heavy chain CDR-H3 amino acid sequence of mouse antibody clone C
CHYDYERVFDY

SEQ ID NO: 4 - protein, 25 aa, synthetic construct
  REGION 1..25: note = Variable region heavy chain framework HC-FR1 amino acid sequence of mouse antibody clone C
EVQLVESGGGLVQPGGSLKLSCAAS

SEQ ID NO: 5 - protein, 14 aa, synthetic construct
  REGION 1..14: note = Variable region heavy chain framework HC-FR2 amino acid sequence of mouse antibody clone C
WVRQTPDKRLELVA

SEQ ID NO: 6 - protein, 32 aa, synthetic construct
  REGION 1..32: note = Variable region heavy chain framework HC-FR3 amino acid sequence of mouse antibody clone C
RFTISRDNDKNSLYLQMSSLKSEDTAMYYCVR

SEQ ID NO: 7 - protein, 11 aa, synthetic construct
  REGION 1..11: note = Variable region heavy chain framework HC-FR4 amino acid sequence of mouse antibody clone C
WGQGTTLTVSS

SEQ ID NO: 8 - protein, 11 aa, synthetic construct
  REGION 1..11: note = Variable light chain CDR-L1 amino acid sequence of mouse antibody clone C
KANDHINNWLA

SEQ ID NO: 9 - protein, 7 aa, synthetic construct
  REGION 1..7: note = Variable light chain CDR-L2 amino acid sequence of mouse antibody clone C
GSTSLET

SEQ ID NO: 10 - protein, 9 aa, synthetic construct
  REGION 1..9: note = Variable light chain CDR-L3 amino acid sequence of mouse antibody clone C
QQYWSTPFT

SEQ ID NO: 11 - protein, 23 aa, synthetic construct
  REGION 1..23: note = Variable region light chain framework LC-FR1 amino acid sequence of mouse antibody clone C1
DIQMTQSSSYLSVSLGGRVTITC

SEQ ID NO: 12 - protein, 15 aa, synthetic construct
  REGION 1..15: note = Variable region light chain framework LC-FR2 amino acid sequence of mouse antibody clone C
WYQQKPGNAPRLLIS

SEQ ID NO: 13 - protein, 32 aa, synthetic construct
  REGION 1..32: note = Variable region light chain framework LC-FR3 amino acid sequence of mouse antibody clone C
GVPSRFSGSGSGKDYTLSITSLQTEDVATYYC

SEQ ID NO: 14 - protein, 10 aa, synthetic construct
  REGION 1..10: note = Variable region light chain framework LC-FR4 amino acid sequence of mouse antibody clone C
FGSGTKLEIK

SEQ ID NO: 15 - protein, 120 aa, synthetic construct
  REGION 1..120: note = Caninized variable heavy chain amino acid sequence of mouse antibody clone C
EVQLVESGGGLVKPGGSLRLSCAASGFTFSSYGMSWVRQAPGKRLELVAIINSNGGSTYYPDSVKGRFTFSLDTSKNTLYLQMNSLRAEDTAMYYCVRCHYDYERVFDYWGQGTLVTVSS

SEQ ID NO: 16 - protein, 107 aa, synthetic construct
  REGION 1..107: note = Caninized variable light chain amino acid sequence of mouse antibody clone C
DIQMTQSPASVSGSLGDKVSITCKANDHINNWLAWYQQLPGNAPRLLISGSTSLESGVPDRFSGSKSGSSFTLTISGLQPEDFATYYCQQYWSTPFTFGSGTKVEIK

SEQ ID NO: 17 - protein, 450 aa, synthetic construct
  REGION 1..450: note = Caninized heavy chain sequence from mouse antibody clone C and canine IgG-A
EVQLVESGGGLVKPGGSLRLSCAASGFTFSSYGMSWVRQAPGKRLELVAIINSNGGSTYYPDSVKGRFTFSLDTSKNTLYLQMNSLRAEDTAMYYCVRCHYDYERVFDYWGQGTLVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 18 - protein, 455 aa, synthetic construct
  REGION 1..455: note = Caninized heavy chain sequence from mouse antibody clone C and canine IgG-B
EVQLVESGGGLVKPGGSLRLSCAASGFTFSSYGMSWVRQAPGKRLELVAIINSNGGSTYYPDSVKGRFTFSLDTSKNTLYLQMNSLRAEDTAMYYCVRCHYDYERVFDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 19 - protein, 453 aa, synthetic construct
  REGION 1..453: note = Caninized heavy chain sequence from mouse antibody clone C and canine IgG-C
EVQLVESGGGLVKPGGSLRLSCAASGFTFSSYGMSWVRQAPGKRLELVAIINSNGGSTYYPDSVKGRFTFSLDTSKNTLYLQMNSLRAEDTAMYYCVRCHYDYERVFDYWGQGTLVTVSSASTTAPSVFPLAPSCGSQSGSTVALACLVSGYIPEPVTVSWNSVSLTSGVHTFPSVLQSSGLYSLSSMVTVPSSRWPSETFTCNVAHPATNTKVDKPVAKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 20 - protein, 451 aa, synthetic construct
  REGION 1..451: note = Caninized heavy chain sequence from mouse antibody clone Clone C and canine IgG-D
EVQLVESGGGLVKPGGSLRLSCAASGFTFSSYGMSWVRQAPGKRLELVAIINSNGGSTYYPDSVKGRFTFSLDTSKNTLYLQMNSLRAEDTAMYYCVRCHYDYERVFDYWGQGTLVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 21 - protein, 217 aa, synthetic construct
  REGION 1..217: note = Caninized light chain sequence from mouse antibody clone C and canine light chain constant region
DIQMTQSPASVSGSLGDKVSITCKANDHINNWLAWYQQLPGNAPRLLISGSTSLESGVPDRFSGSKSGSSFTLTISGLQPEDFATYYCQQYWSTPFTFGSGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 22 - protein, 131 aa, unidentified
  REGION 1..131: note = Mature canine IL17A amino acid sequence
AGIAFPQNPGCRNTEDKNFPQHVKVNLNILNRNTNSRRPSDYYNRSTSPWNLHRNEDPERYPSVIWEAKCRHLGCVNNEGNINYHMNSVPIQQEILVLRRESQHCPHSFRLEKMLVAVGCTCVTPIVRHVA

SEQ ID NO: 23 - protein, 12 aa, unidentified
  REGION 1..12: note = Canine IL17A epitope, minimal sequence
RHLGCVNNEGNI

SEQ ID NO: 24 - protein, 107 aa, synthetic construct
  REGION 1..107: note = Variable light chain amino acid sequence of mouse antibody clone C
DIQMTQSSSYLSVSLGGRVTITCKANDHINNWLAWYQQKPGNAPRLLISGSTSLETGVPSRFSGSGSGKDYTLSITSLQTEDVATYYCQQYWSTPFTFGSGTKLEIK

SEQ ID NO: 25 - protein, 120 aa, synthetic construct
  REGION 1..120: note = Variable heavy chain amino acid sequence of mouse antibody clone C
EVQLVESGGGLVQPGGSLKLSCAASGFTFSSYGMSWVRQTPDKRLELVAIINSNGGSTYYPDSVKGRFTISRDNDKNSLYLQMSSLKSEDTAMYYCVRCHYDYERVFDYWGQGTTLTVSS

SEQ ID NO: 26 - protein, 217 aa, synthetic construct
  REGION 1..217: note = Chimeric variable light chain of mouse antibody clone C and canine light chain constant region
DIQMTQSSSYLSVSLGGRVTITCKANDHINNWLAWYQQKPGNAPRLLISGSTSLETGVPSRFSGSGSGKDYTLSITSLQTEDVATYYCQQYWSTPFTFGSGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 27 - protein, 455 aa, synthetic construct
  REGION 1..455: note = Chimeric variable heavy chain of mouse antibody clone C and canine IgG-B
EVQLVESGGGLVQPGGSLKLSCAASGFTFSSYGMSWVRQTPDKRLELVAIINSNGGSTYYPDSVKGRFTISRDNDKNSLYLQMSSLKSEDTAMYYCVRCHYDYERVFDYWGQGTTLTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 28 - protein, 155 aa, unidentified
  REGION 1..155: note = Canine IL17A precursor amino acid sequence
MTLVTTSSMFQSLLLLLSLVAIIKAGIAFPQNPGCRNTEDKNFPQHVKVNLNILNRNTNSRRPSDYYNRSTSPWNLHRNEDPERYPSVIWEAKCRHLGCVNNEGNINYHMNSVPIQQEILVLRRESQHCPHSFRLEKMLVAVGCTCVTPIVRHVA

SEQ ID NO: 29 - protein, 153 aa, Equus caballus
MAPLRTSSVSLLLLLSLVAIVKAGIVIPQNPECPNTGDKNFPQNVKINLNVLNRKTNSRRASDYHNRSTSPWNLHRNEDPERYPSVIWEAKCRHLGCVNAEGKVDFHMNSVPIQQEILVLRRESQNCPHSFQLEKMLVAVGCTCVTPIVRHMG

SEQ ID NO: 30 - protein, 155 aa, Felis catus
MAPGTTSSMFPSLLLLLCLMAIVRTGIAFPQNPGCPTTEDKNFPQHVKVNVNILNGNKSSRRPLDYYRRSTSPWSLHRNEDPERYPSVIWEAKCLHWGCVNTEGKEDHHMNSVPIQQEILVLRRESRHCPHSFRLEKMLVTVGCTCVTPIVRHVV

SEQ ID NO: 31 - protein, 157 aa, unidentified
  REGION 1..157: note = Canine IL17F precursor amino acid sequence
MAILRNIAMVKSLLLLVLGLTLLSEVAARKHLKAGETALCPPLEDNSVRVDIRILRQNRGISISNDFQNRSSSPWDYNITRDPHRFPSEIAEAQCRHSGCINAEGQEDSSMNSVPIQQEFLVLRREPQGCSRSFRLEKVLVTVGCTCVTPIVRYVRA

SEQ ID NO: 32 - protein, 822 aa, unidentified
  REGION 1..822: note = Canine IL17A receptor (IL17Ra) amino acid sequence
MGRLGEGLNCTVKNSTCLDDSWIHPRNLTPSSPKDVQVHLDFAQTQHGDLLPIIGIRWTLQTDASILFLEGAELSVLQLNTNERVCVKFEFLSKLKHHHKRWHFTFSHFVVEPGQEYEVTVHHLPKPIPDGDPNHQSKNFLVPGCEDPRMRMTTPCVSSGSLWDPNITAEALEAHQLQVHFTLWNESAQYQILLTSFPHTENRSCFHRVLMVPEPTLKEHHQRANIMLTGSSSNWCCRHQVQIQPFFSSCLNDCLRHSVTVPCPEIPDAPVSIADYIPLWAYGFITGIAILLVGSVILLIVCMAWRLPGSHCEKYGNDSKYTDIQPKTSLTPPPLKPRKVWIVYSADHPLYVDVVLKFAQFLLTVCGTEVALDLLEEQVISEVGVMTWVGRQKQEMVETNSKIIILCSRGTRAKWQAILGWEEPAVQLRCDRWKPSGDLFTAAMNMILPDFKKPACFGTYIICYFRDISSESDIPDLFNITSRYPLMDKFEEVYFRIQDLEMFEPGRMHRVGELTGENYLQSPSGWQLKEAVERFREWQVRCPDWFERENLGSADDQDLPSLDEEVFEEPLLPPGRGIVKQKPLVHEPAPEGCLVIDLLVGEEGRGPSRLEPQLQPQGELMAQTLQTVVFPVKEVPSAQAVEPVPHTVESSTAGRLAVVEGDEACPLLEGCGPWRNSVLCLPMDSEEPPLCRTPMASPSYLPEDVREQLEGLMFSLLEQSLSCQAQEGWDRAAVALKDFRTPYEEEQRQSVQSDQGYISRSSPQPPEGLMEMEEEEAEQDLGKSAKQLSPEDLESLRSLQRQLFFQELQTNSGWDSVELEVP

SEQ ID NO: 33 - protein, 279 aa, unidentified
  REGION 1..279: note = Canine IL17A receptor (IL17Ra) ECD (binding domain fragment) amino acid sequence
MGRLGEGLNCTVKNSTCLDDSWIHPRNLTPSSPKDVQVHLDFAQTQHGDLLPIIGIRWTLQTDASILFLEGAELSVLQLNTNERVCVKFEFLSKLKHHHKRWHFTFSHFVVEPGQEYEVTVHHLPKPIPDGDPNHQSKNFLVPGCEDPRMRMTTPCVSSGSLWDPNITAEALEAHQLQVHFTLWNESAQYQILLTSFPHTENRSCFHRVLMVPEPTLKEHHQRANIMLTGSSSNWCCRHQVQIQPFFSSCLNDCLRHSVTVPCPEIPDAPVSIADYIPL

SEQ ID NO: 34 - protein, 112 aa, synthetic construct
  REGION 1..112: note = Variable light chain amino acid sequence of mouse antibody clone A
DVVMTQTPLSLPVSLGDQASISCRSSQSLVHSNGNTYLHWYLQRPGQSPNLLIYKVSNRFSGVPDRFSGSGSGTDFTLKISRVEAEDLGVYFCSQSTHVPFTFGSGTKLEIK

SEQ ID NO: 35 - protein, 120 aa, synthetic construct
  REGION 1..120: note = Variable heavy chain amino acid sequence of mouse antibody clone A
QVQLKESGPGLVAPSQSLSITCTISGFSLTSNGVHWVRQSPGKDLEWLVVIWSDGTTTYNSDFKSRLSISKDNSKSQVFLKMNSLQTDDTAMYYCARHYDWGYYYAMDYWGQGTSVTVSS

SEQ ID NO: 36 - protein, 110 aa, synthetic construct
  REGION 1..110: note = Variable light chain amino acid sequence of mouse antibody clone D
DIVLTQSPASLAVSLGQRATISYRASKSVSTSGYSYMHWNQQKPGQPPRLLIYLVSNLESGVPARFSGSGSGTDFTLNIHPVEEEDAATYYCQHIRELYTFGGGTKLEIK

SEQ ID NO: 37 - protein, 122 aa, synthetic construct
  REGION 1..122: note = Variable heavy chain amino acid sequence of mouse antibody clone D
EVQLQQSGPELVKTGASVKISCKASGYSFTYYYMHWVKQSHGKSLEWIGYISCFNGDTNYNQEFKDKATFTADTSSSTAYMQFNSLTSEDSAVYYCARGLSTLITEGWFAYWGQGTLVTVSA

SEQ ID NO: 38 - protein, 112 aa, synthetic construct
  REGION 1..112: note = Variable light chain amino acid sequence of mouse antibody clone E
DVVMTQTPLSLPVSLGDQASISCRSSQSLVHSNGNTYFHWYLQKPGQSPKLLIYKVSNRFSGVPDRFSGSGSGTDFTLKISRVEAEDLGVYFCSQSTHAPFTFGSGTKLEIK

SEQ ID NO: 39 - protein, 120 aa, synthetic construct
  REGION 1..120: note = Variable heavy chain amino acid sequence of mouse antibody clone E
QVQLKESGPGLVAPSQSLSITCTISGFSLTSNGVHWVRQPPGKGLEWLVVIWSDGTTTYNSALKSRLSISKDNSKSQVFLKMNSLQTDDTAMYYCARHYDRGYYYAMDYWGQGTSVTVSS

SEQ ID NO: 40 - protein, 514 aa, Homo sapiens
SLRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPDITVETLEAHQLRVSFTLWNESTHYQILLTSFPHMENHSCFEHMHHIPAPRPEEFHQRSDVTLTLRNLKGCCRHQVQIQPFFSSCLNDCLRHSATVSCPEMPDTPEPIPDGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 41 - protein, 516 aa, synthetic construct
  REGION 1..516: note = Canine IL17Ra ECD-canine IgG-B-Fc
MGRLGEGLNCTVKNSTCLDDSWIHPRNLTPSSPKDVQVHLDFAQTQHGDLLPIIGIRWTLQTDASILFLEGAELSVLQLNTNERVCVKFEFLSKLKHHHKRWHFTFSHFVVEPGQEYEVTVHHLPKPIPDGDPNHQSKNFLVPGCEDPRMRMTTPCVSSGSLWDPNITAEALEAHQLQVHFTLWNESAQYQILLTSFPHTENRSCFHRVLMVPEPTLKEHHQRANIMLTGSSSNWCCRHQVQIQPFFSSCLNDCLRHSVTVPCPEIPDAPVSIADYIGSPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 42 - protein, 545 aa, synthetic construct
  REGION 1..545: note = Canine IL17Ra ECD-huFc-polyHis
MDMRVPAQLLGLLLLWLRGARCMGRLGEGLNCTVKNSTCLDDSWIHPRNLTPSSPKDVQVHLDFAQTQHGDLLPIIGIRWTLQTDASILFLEGAELSVLQLNTNERVCVKFEFLSKLKHHHKRWHFTFSHFVVEPGQEYEVTVHHLPKPIPDGDPNHQSKNFLVPGCEDPRMRMTTPCVSSGSLWDPNITAEALEAHQLQVHFTLWNESAQYQILLTSFPHTENRSCFHRVLMVPEPTLKEHHQRANIMLTGSSSNWCCRHQVQIQPFFSSCLNDCLRHSVTVPCPEIPDAPVSIADYIGSENLYFQGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 43 - protein, 525 aa, synthetic construct
  REGION 1..525: note = Feline IL17Ra ECD-feline IgG-2-Fc
SPRLLDYPAPVCSQQGLNCVVKNSTCLDDSWIHLRNLTPSSPKDVQVHLDFVQTQHGDLLPVAGIRWTLQTDASILYLEGAELSVLQLNTNERLCVKFEFLTRLKHHHKRWHFTFSHFVVEPGQEYEVTVHHLPKPIPDGDPNHQSRNFPVPGCEDPRMKMITPCVGSGSLWDPNITVETLEARQLWVSFTLWNESTHYQILLTSFPHTENHSCFQHTLMVPEPAYQDSRQRSNVTLTLSDSNWCCRHRVQIQPFFSSCLNDCLRHSITVPCPEIPDPPVSIADYIGSPKTASTIESKTGECPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 44 - protein, 516 aa, synthetic construct
  REGION 1..516: note = Equine IL17Ra ECD-equine IgG-2-Fc
SPRLLEHPAPVCSQQGLNCTVKNSTCLDDSWLHPPHLTPSSPKDVQIQLHFAHTQQGDLLPVIHIEWTLQTDASILYLEGAELSVLQLSTNERLCVTFEFLSRLKHHHKRWRFTFAHFVVEPGQEYEVTVHHLPKPFPHGDPNHQSRNFLVPDCMDPRMRITTPCVSSGSLWDPNITVETLEAHRLRVDFTLWNESARYQILLSSFPHMENQSCFDDVQNILKHTPEASHQRANITLTLSDFNWCCRHHVQIQPFFSSCLNDCLRHTVTVPCPEIPDTPDSTADYMGSDMSKCPKCPAPELLGGPSVFIFPPNPKDALMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHFTKTDISESLGK

SEQ ID NO: 45 - protein, 237 aa, unidentified
  REGION 1..237: note = Canine IgG-B-Fc
PKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 46 - protein, 235 aa, unidentified
  REGION 1..235: note = Canine IgG-C-Fc
AKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 47 - protein, 237 aa, synthetic construct
  REGION 1..237: note = Canine IgG-B-Fc variant 1 (C1q binding mutant)
PKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCRVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 48 - protein, 235 aa, synthetic construct
  REGION 1..235: note = Canine IgG-C-Fc variant 1 (C1q binding mutant)
AKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCRVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 49 - protein, 237 aa, synthetic construct
  REGION 1..237: note = Canine IgG-B-Fc variant 2 (CD16 binding mutant 1)
PKRENGRVPRPPDCPKCPAPEMLGGPSVFIFPPKPKDTLLIARTPEVTCVVVDLGPEDPEVQISWFVDGKQMQTAKTQPREEQFNGTYRVVSVLPIGHQDWLKGKQFTCKVNNIGLPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 50 - protein, 235 aa, synthetic construct
  REGION 1..235: note = Canine IgG-C-Fc variant 2 (CD16 binding mutant 1)
AKECECKCNCNNCPCPGCGLLGGPSVFIFPPKPKDILVTARTPTVTCVVVDLGPENPEVQISWFVDSKQVQTANTQPREEQSNGTYRVVSVLPIGHQDWLSGKQFKCKVNNIGLPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 51 - protein, 14 aa, synthetic construct
  REGION 1..14: note = Canine IL17A epitope C, expanded sequence
CRHLGCVNNEGNIN

SEQ ID NO: 52 - protein, 10 aa, synthetic construct
  REGION 1..10: note = Variable heavy chain CDR-H1 amino acid sequence of mouse antibody clone A
GFSLTSNGVH

SEQ ID NO: 53 - protein, 19 aa, synthetic construct
  REGION 1..19: note = Variable heavy chain CDR-H2 amino acid sequence of mouse antibody clone A
WLVVIWSDGTTTYNSDFKS

SEQ ID NO: 54 - protein, 14 aa, synthetic construct
  REGION 1..14: note = Variable heavy chain CDR-H3 amino acid sequence of mouse antibody clone A
ARHYDWGYYYAMDY

SEQ ID NO: 55 - protein, 25 aa, synthetic construct
  REGION 1..25: note = Variable region heavy chain framework HC-FR1 amino acid sequence of mouse antibody clone A
QVQLKESGPGLVAPSQSLSITCTIS

SEQ ID NO: 56 - protein, 11 aa, synthetic construct
  REGION 1..11: note = Variable region heavy chain framework HC-FR2 amino acid sequence of mouse antibody clone A
WVRQSPGKDLE

SEQ ID NO: 57 - protein, 30 aa, synthetic construct
  REGION 1..30: note = Variable region heavy chain framework HC-FR3 amino acid sequence of mouse antibody clone A
RLSISKDNSKSQVFLKMNSLQTDDTAMYYC

SEQ ID NO: 58 - protein, 11 aa, synthetic construct
  REGION 1..11: note = Variable region heavy chain framework HC-FR4 amino acid sequence of mouse antibody clone A
WGQGTSVTVSS

SEQ ID NO: 59 - protein, 17 aa, synthetic construct
  REGION 1..17: note = Variable light chain CDR-L1 amino acid sequence of mouse antibody clone A
SSQSLVHSNGNTYLHWY

SEQ ID NO: 60 - protein, 11 aa, synthetic construct
  REGION 1..11: note = Variable light chain CDR-L2 amino acid sequence of mouse antibody clone A
LLIYKVSNRFS

SEQ ID NO: 61 - protein, 9 aa, synthetic construct
  REGION 1..9: note = Variable light chain CDR-L3 amino acid sequence of mouse antibody clone A
SQSTHVPFT

SEQ ID NO: 62 - protein, 24 aa, synthetic construct
  REGION 1..24: note = Variable region light chain framework LC-FR1 amino acid sequence of mouse antibody clone A
DVVMTQTPLSLPVSLGDQASISCR

SEQ ID NO: 63 - protein, 9 aa, synthetic construct
  REGION 1..9: note = Variable region light chain framework LC-FR2 amino acid sequence of mouse antibody clone A
LQRPGQSPN

SEQ ID NO: 64 - protein, 32 aa, synthetic construct
  REGION 1..32: note = Variable region light chain framework LC-FR3 amino acid sequence of mouse antibody clone A
GVPDRFSGSGSGTDFTLKISRVEAEDLGVYFC

SEQ ID NO: 65 - protein, 10 aa, synthetic construct
  REGION 1..10: note = Variable region light chain framework LC-FR4 amino acid sequence of mouse antibody clone A
FGSGTKLEIK

SEQ ID NO: 66 - protein, 10 aa, synthetic construct
  REGION 1..10: note = Variable heavy chain CDR-H1 amino acid sequence of mouse antibody clone E
GFSLTSNGVH

SEQ ID NO: 67 - protein, 19 aa, synthetic construct
  REGION 1..19: note = Variable heavy chain CDR-H2 amino acid sequence of mouse antibody clone E
WLVVIWSDGTTTYNSALKS

SEQ ID NO: 68 - protein, 14 aa, synthetic construct
  REGION 1..14: note = Variable heavy chain CDR-H3 amino acid sequence of mouse antibody clone E
ARHYDRGYYYAMDY

SEQ ID NO: 69 - protein, 25 aa, synthetic construct
  REGION 1..25: note = Variable region heavy chain framework HC-FR1 amino acid sequence of mouse antibody clone E
QVQLKESGPGLVAPSQSLSITCTIS

SEQ ID NO: 70 - protein, 11 aa, synthetic construct
  REGION 1..11: note = Variable region heavy chain framework HC-FR2 amino acid sequence of mouse antibody clone E
WVRQPPGKGLE

SEQ ID NO: 71 - protein, 30 aa, synthetic construct; identical to SEQ ID NO: 57
  REGION 1..30: note = Variable region heavy chain framework HC-FR3 amino acid sequence of mouse antibody clone E

SEQ ID NO: 72 - protein, 11 aa, synthetic construct
  REGION 1..11: note = Variable region heavy chain framework HC-FR4 amino acid sequence of mouse antibody clone E
WGQGTSVTVSS

SEQ ID NO: 73 - protein, 18 aa, synthetic construct
  REGION 1..18: note = Variable light chain CDR-L1 amino acid sequence of mouse antibody clone E
RSSQSLVHSNGNTYFHWY

SEQ ID NO: 74 - protein, 11 aa, synthetic construct
  REGION 1..11: note = Variable light chain CDR-L2 amino acid sequence of mouse antibody clone E
LLIYKVSNRFS

SEQ ID NO: 75 - protein, 9 aa, synthetic construct
  REGION 1..9: note = Variable light chain CDR-L3 amino acid sequence of mouse antibody clone E
SQSTHAPFT

SEQ ID NO: 76 - protein, 23 aa, synthetic construct
  REGION 1..23: note = Variable region light chain framework LC-FR1 amino acid sequence of mouse antibody clone E
DVVMTQTPLSLPVSLGDQASISC

SEQ ID NO: 77 - protein, 9 aa, synthetic construct
  REGION 1..9: note = Variable region light chain framework LC-FR2 amino acid sequence of mouse antibody clone E
LQKPGQSPK

SEQ ID NO: 78 - protein, 32 aa, synthetic construct; identical to SEQ ID NO: 64
  REGION 1..32: note = Variable region light chain framework LC-FR3 amino acid sequence of mouse antibody clone E

SEQ ID NO: 79 - protein, 10 aa, synthetic construct
  REGION 1..10: note = Variable region light chain framework LC-FR4 amino acid sequence of mouse antibody clone E
FGSGTKLEIK

SEQ ID NO: 80 - protein, 10 aa, synthetic construct
  REGION 1..10: note = Variable heavy chain CDR-H1 amino acid sequence of mouse antibody clone D
GYSFTYYYMH

SEQ ID NO: 81 - protein, 20 aa, synthetic construct
  REGION 1..20: note = Variable heavy chain CDR-H2 amino acid sequence of mouse antibody clone D
WIGYISCFNGDTNYNQEFKD

SEQ ID NO: 82 - protein, 15 aa, synthetic construct
  REGION 1..15: note = Variable heavy chain CDR-H3 amino acid sequence of mouse antibody clone D
ARGLSTLITEGWFAY

SEQ ID NO: 83 - protein, 25 aa, synthetic construct
  REGION 1..25: note = Variable region heavy chain framework HC-FR1 amino acid sequence of mouse antibody clone D
EVQLQQSGPELVKTGASVKISCKAS

SEQ ID NO: 84 - protein, 11 aa, synthetic construct
  REGION 1..11: note = Variable region heavy chain framework HC-FR2 amino acid sequence of mouse antibody clone D
WVKQSHGKSLE

SEQ ID NO: 85 - protein, 30 aa, synthetic construct
  REGION 1..30: note = Variable region heavy chain framework HC-FR3 amino acid sequence of mouse antibody clone D
KATFTADTSSSTAYMQFNSLTSEDSAVYYC

SEQ ID NO: 86 - protein, 11 aa, synthetic construct
  REGION 1..11: note = Variable region heavy chain framework HC-FR4 amino acid sequence of mouse antibody clone D
WGQGTLVTVSA

SEQ ID NO: 87 - protein, 17 aa, synthetic construct
  REGION 1..17: note = Variable light chain CDR-L1 amino acid sequence of mouse antibody clone D
RASKSVSTSGYSYMHWN

SEQ ID NO: 88 - protein, 11 aa, synthetic construct
  REGION 1..11: note = Variable light chain CDR-L2 amino acid sequence of mouse antibody clone D
LLIYLVSNLES

SEQ ID NO: 89 - protein, 8 aa, synthetic construct
  REGION 1..8: note = Variable light chain CDR-L3 amino acid sequence of mouse antibody clone D
QHIRELYT

SEQ ID NO: 90 - protein, 23 aa, synthetic construct
  REGION 1..23: note = Variable region light chain framework LC-FR1 amino acid sequence of mouse antibody clone D
DIVLTQSPASLAVSLGQRATISY

SEQ ID NO: 91 - protein, 9 aa, synthetic construct
  REGION 1..9: note = Variable region light chain framework LC-FR2 amino acid sequence of mouse antibody clone D
QQKPGQPPR

SEQ ID NO: 92 - protein, 32 aa, synthetic construct
  REGION 1..32: note = Variable region light chain framework LC-FR3 amino acid sequence of mouse antibody clone D
GVPARFSGSGSGTDFTLNIHPVEEEDAATYYC

SEQ ID NO: 93 - protein, 10 aa, synthetic construct
  REGION 1..10: note = Variable region light chain framework LC-FR4 amino acid sequence of mouse antibody clone D
FGGGTKLEIK

SEQ ID NO: 94 - protein, 261 aa, synthetic construct
  REGION 1..261: note = Truncated canine IL17Ra ECD
LGEGLNCTVKNSTCLDDSWIHPRNLTPSSPKDVQVHLDFAQTQHGDLLPIIGIRWTLQTDASILFLEGAELSVLQLNTNERVCVKFEFLSKLKHHHKRWHFTFSHFVVEPGQEYEVTVHHLPKPIPDGDPNHQSKNFLVPGCEDPRMRMTTPCVSSGSLWDPNITAEALEAHQLQVHFTLWNESAQYQILLTSFPHTENRSCFHRVLMVPEPTLKEHHQRANIMLTGSSSNWCCRHQVQIQPFFSSCLNDCLRHSVTVPCP

SEQ ID NO: 95 - protein, 780 aa, unidentified
  REGION 1..780: note = IL4R/IL13R-canine IgG-B
MAVLGLLFCLVTFPSCVLSTETQPPVTNLSVSVENLCTVIWTWDPPEGASPNCTLRYFSHFDNKQDKKIAPETHRSKEVPLNERICLQVGSQCSTNESDNPSILVEKCTPPPEGDPESAVTELQCVWHNLSYMKCTWLPGRNTSPDTNYTLYYWHSSLGKILQCEDIYREGQHIGCSFALTNLKDSSFEQHSVQIVVKDNAGKIRPSFNIVPLTSHVKPDPPHIKRLFFQNGNLYVQWKNPQNFYSRCLSYQVEVNNSQTETNDIFYVEEAKCQNSEFEGNLEGTICFMVPGVLPDTLNTVRIRVRTNKLCYEDDKLWSNWSQAMSIGENTDPTGGGSGSGSVKVLHEPSCFSDYISTSVCQWKMDHPTNCSAELRLSYQLDFMGSENHTCVPENREDSVCVCSMPIDDAVEADVYQLDLWAGQQLLWSGSFQPSKHVKPRTPGNLTVHPNISHTWLLMWTNPYPTENHLHSELTYMVNVSNDNDPEDFKVYNVTYMGPTLRLAASTLKSGASYSARVRAWAQTYNSTWSDWSPSTTWLNYYEPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 96 - protein, 780 aa, synthetic construct
  REGION 1..780: note = IL4R/IL13R-canine IgG-B variant 1 (C1q binding mutant)
MAVLGLLFCLVTFPSCVLSTETQPPVTNLSVSVENLCTVIWTWDPPEGASPNCTLRYFSHFDNKQDKKIAPETHRSKEVPLNERICLQVGSQCSTNESDNPSILVEKCTPPPEGDPESAVTELQCVWHNLSYMKCTWLPGRNTSPDTNYTLYYWHSSLGKILQCEDIYREGQHIGCSFALTNLKDSSFEQHSVQIVVKDNAGKIRPSFNIVPLTSHVKPDPPHIKRLFFQNGNLYVQWKNPQNFYSRCLSYQVEVNNSQTETNDIFYVEEAKCQNSEFEGNLEGTICFMVPGVLPDTLNTVRIRVRTNKLCYEDDKLWSNWSQAMSIGENTDPTGGGSGSGSVKVLHEPSCFSDYISTSVCQWKMDHPTNCSAELRLSYQLDFMGSENHTCVPENREDSVCVCSMPIDDAVEADVYQLDLWAGQQLLWSGSFQPSKHVKPRTPGNLTVHPNISHTWLLMWTNPYPTENHLHSELTYMVNVSNDNDPEDFKVYNVTYMGPTLRLAASTLKSGASYSARVRAWAQTYNSTWSDWSPSTTWLNYYEPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCRVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 97 - protein, 273 aa, Homo sapiens
SLRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPDITVETLEAHQLRVSFTLWNESTHYQILLTSFPHMENHSCFEHMHHIPAPRPEEFHQRSDVTLTLRNLKGCCRHQVQIQPFFSSCLNDCLRHSATVSCP

SEQ ID NO: 98 - protein, 286 aa, unidentified
  REGION 1..286: note = Feline IL17Ra ECD
SPRLLDYPAPVCSQQGLNCVVKNSTCLDDSWIHLRNLTPSSPKDVQVHLDFVQTQHGDLLPVAGIRWTLQTDASILYLEGAELSVLQLNTNERLCVKFEFLTRLKHHHKRWHFTFSHFVVEPGQEYEVTVHHLPKPIPDGDPNHQSRNFPVPGCEDPRMKMITPCVGSGSLWDPNITVETLEARQLWVSFTLWNESTHYQILLTSFPHTENHSCFQHTLMVPEPAYQDSRQRSNVTLTLSDSNWCCRHRVQIQPFFSSCLNDCLRHSITVPCPEIPDPPVSIADYI

SEQ ID NO: 99 - protein, 286 aa, unidentified
  REGION 1..286: note = Equine IL17Ra ECD
SPRLLEHPAPVCSQQGLNCTVKNSTCLDDSWLHPPHLTPSSPKDVQIQLHFAHTQQGDLLPVIHIEWTLQTDASILYLEGAELSVLQLSTNERLCVTFEFLSRLKHHHKRWRFTFAHFVVEPGQEYEVTVHHLPKPFPHGDPNHQSRNFLVPDCMDPRMRITTPCVSSGSLWDPNITVETLEAHRLRVDFTLWNESARYQILLSSFPHMENQSCFDDVQNILKHTPEASHQRANITLTLSDFNWCCRHHVQIQPFFSSCLNDCLRHTVTVPCPEIPDTPDSTADYM

SEQ ID NO: 100 - protein, 174 aa, synthetic construct
  REGION 1..174: note = Mouse monoclonal antibody Clone C variable light chain
MKFPSQLLLFLLFRITGIICDIQMTQSSSYLSVSLGGRVTITCKANDHINNWLAWYQQKPGNAPRLLISGSTSLETGVPSRFSGSGSGKDYTLSITSLQTEDVATYYCQQYWSTPFTFGSGTKLEIKRADAAPTVSIFPPSSEQLTSGGASVVLGVIMVIAVSCVKLLSAHNST

SEQ ID NO: 101 - protein, 161 aa, synthetic construct
  REGION 1..161: note = Mouse monoclonal antibody Clone E variable light chain
MKLPVRLLVLMFWIPASNSDVVMTQTPLSLPVSLGDQASISCRSSQSLVHSNGNTYFHWYLQKPGQSPKLLIYKVSNRFSGVPDRFSGSGSGTDFTLKISRVEAEDLGVYFCSQSTHAPFTFGSGTKLEIKRADAAPTVSIFPPSSEQLTSGGASVVSRAN

SEQ ID NO: 102 - protein, 162 aa, synthetic construct
  REGION 1..162: note = Mouse monoclonal antibody Clone A variable light chain
MKLPVRLLVLMFWIPASSSDVVMTQTPLSLPVSLGDQASISCRSSQSLVHSNGNTYLHWYLQRPGQSPNLLIYKVSNRFSGVPDRFSGSGSGTDFTLKISRVEAEDLGVYFCSQSTHVPFTFGSGTKLEIKRADAAPTVSIFPPSSEQLTSGGASVVCKGEF

SEQ ID NO: 103 - protein, 140 aa, synthetic construct
  REGION 1..140: note = Mouse monoclonal antibody Clone D variable light chain
METDTLLLWVLLLWVPGSTGDIVLTQSPASLAVSLGQRATISYRASKSVSTSGYSYMHWNQQKPGQPPRLLIYLVSNLESGVPARFSGSGSGTDFTLNIHPVEEEDAATYYCQHIRELYTFGGGTKLEIKRADAAPTVSI

SEQ ID NO: 104 - protein, 157 aa, synthetic construct
  REGION 1..157: note = Mouse monoclonal antibody Clone D variable heavy chain
MGWIWIFLFLLSGTAGVHSEVQLQQSGPELVKTGASVKISCKASGYSFTYYYMHWVKQSHGKSLEWIGYISCFNGDTNYNQEFKDKATFTADTSSSTAYMQFNSLTSEDSAVYYCARGLSTLITEGWFAYWGQGTLVTVSAAKTTPPSVYPLAPGSA

SEQ ID NO: 105 - protein, 174 aa, synthetic construct
  REGION 1..174: note = Mouse monoclonal antibody Clone C variable heavy chain
MNLGLSFIFLALILKGVQCEVQLVESGGGLVQPGGSLKLSCAASGFTFSSYGMSWVRQTPDKRLELVAIINSNGGSTYYPDSVKGRFTISRDNDKNSLYLQMSSLKSEDTAMYYCVRCHYDYERVFDYWGQGTTLTVSSAKTTPPSVYPLAPGSAAQTNSMVTLGCLVKGYFPE

SEQ ID NO: 106 - protein, 172 aa, synthetic construct
  REGION 1..172: note = Mouse monoclonal antibody Clone A variable heavy chain
MAVLGLLLCLVTFPSCVLSQVQLKESGPGLVAPSQSLSITCTISGFSLTSNGVHWVRQSPGKDLEWLVVIWSDGTTTYNSDFKSRLSISKDNSKSQVFLKMNSLQTDDTAMYYCARHYDWGYYYAMDYWGQGTSVTVSSAKTTPPSVYPLAPGSAAQTNSMVTLGCLVKGEF

SEQ ID NO: 107 - protein, 172 aa, synthetic construct
  REGION 1..172: note = Mouse monoclonal antibody Clone E variable heavy chain
MAVLGLLLCLVTFPSCVLSQVQLKESGPGLVAPSQSLSITCTISGFSLTSNGVHWVRQPPGKGLEWLVVIWSDGTTTYNSALKSRLSISKDNSKSQVFLKMNSLQTDDTAMYYCARHYDRGYYYAMDYWGQGTSVTVSSAKTTPPSVYPLAPGSAAQTNSMVTLGCLVKGEF

SEQ ID NO: 108 - protein, 7 aa, synthetic construct
  REGION 1..7: note = Variable light chain CDR-L2 v2 amino acid sequence of mouse antibody clone C
GSTSLES

SEQ ID NO: 109 - protein, 16 aa, synthetic construct
  REGION 1..16: note = Variable heavy chain CDR-H2 v2 amino acid sequence of mouse antibody clone A
VIWSDGTTTYNSDFKS

SEQ ID NO: 110 - protein, 14 aa, synthetic construct
  REGION 1..14: note = Variable region heavy chain framework HC-FR2 v2 amino acid sequence of mouse antibody clone A
WVRQSPGKDLEWLV

SEQ ID NO: 111 - protein, 15 aa, synthetic construct
  REGION 1..15: note = Variable light chain CDR-L1 v2 amino acid sequence of mouse antibody clone A
SSQSLVHSNGNTYLH

SEQ ID NO: 112 - protein, 7 aa, synthetic construct
  REGION 1..7: note = Variable light chain CDR-L2 v2 amino acid sequence of mouse antibody clone A
KVSNRFS

SEQ ID NO: 113 - protein, 15 aa, synthetic construct
  REGION 1..15: note = Variable region light chain framework LC-FR2 v2 amino acid sequence of mouse antibody clone A
WYLQRPGQSPNLLIY

SEQ ID NO: 114 - protein, 16 aa, synthetic construct
  REGION 1..16: note = Variable heavy chain CDR-H2 v2amino acid sequence of mouse antibody clone E
VIWSDGTTTYNSALKS

SEQ ID NO: 115 - protein, 14 aa, synthetic construct
  REGION 1..14: note = Variable region heavy chain framework HC-FR2 v2 amino acid sequence of mouse antibody clone E
WVRQPPGKGLEWLV

SEQ ID NO: 116 - protein, 16 aa, synthetic construct
  REGION 1..16: note = Variable light chain CDR-L1 v2 amino acid sequence of mouse antibody clone E
RSSQSLVHSNGNTYFH

SEQ ID NO: 117 - protein, 7 aa, synthetic construct
  REGION 1..7: note = Variable light chain CDR-L2 v2 amino acid sequence of mouse antibody clone E
KVSNRFS

SEQ ID NO: 118 - protein, 15 aa, synthetic construct
  REGION 1..15: note = Variable region light chain framework LC-FR2 v2 amino acid sequence of mouse antibody clone E
WYLQKPGQSPKLLIY

SEQ ID NO: 119 - protein, 17 aa, synthetic construct
  REGION 1..17: note = Variable heavy chain CDR-H2 v2 amino acid sequence of mouse antibody clone D
YISCFNGDTNYNQEFKD

SEQ ID NO: 120 - protein, 14 aa, synthetic construct
  REGION 1..14: note = Variable region heavy chain framework HC-FR2 v2 amino acid sequence of mouse antibody clone D
WVKQSHGKSLEWIG

SEQ ID NO: 121 - protein, 15 aa, synthetic construct
  REGION 1..15: note = Variable light chain CDR-L1 v2 amino acid sequence of mouse antibody clone D
RASKSVSTSGYSYMH

SEQ ID NO: 122 - protein, 7 aa, synthetic construct
  REGION 1..7: note = Variable light chain CDR-L2 v2 amino acid sequence of mouse antibody clone D
LVSNLES

SEQ ID NO: 123 - protein, 15 aa, synthetic construct
  REGION 1..15: note = Variable region light chain framework LC-FR2 v2 amino acid sequence of mouse antibody clone D
WNQQKPGQPPRLLIY